(19) United States
(12) Patent Application Publication
(10) Pub. No.: US 2022/0356490 A1
(43) Pub. Date: Nov. 10, 2022

(54) BACULOVIRUS EXPRESSION SYSTEM

(71) Applicant: BIOVERATIV THERAPEUTICS INC., Waltham, MA (US)

(72) Inventors: Tongyao Liu, Waltham, MA (US); Ajay Maghodia, Waltham, MA (US); Philip Zakas, Waltham, MA (US)

(21) Appl. No.: 17/576,561

(22) Filed: Jan. 14, 2022

Related U.S. Application Data
(63) Continuation of application No. PCT/US2021/047218, filed on Aug. 23, 2021.
(60) Provisional application No. 63/069,073, filed on Aug. 23, 2020.

(51) Int. Cl. C12N 15/866; C12N 15/82

(52) U.S. Cl. CPC C12N 15/866; C12N 15/8209; C12N 2710/14033; C12N 2710/14144

(57) ABSTRACT

Provided herein is a baculovirus expression vector system for the production of a desired protein. In one aspect, the desired protein is a closed-ended DNA (ceDNA) molecule comprising wild-type and/or truncated inverted terminal repeats derived from a genome of a member of the viral family Parvoviridae.

### RELATED APPLICATIONS

This application is a continuation of International Patent Application No. PCT/US2021/047218, filed Aug. 23, 20201, which claims priority to U.S. Provisional Patent Application No. 63/069,073, filed Aug. 23, 2020, the entire disclosures of which are hereby incorporated by reference in their entirety.

### REFERENCE TO SEQUENCE LISTING SUBMITTED ELECTRONICALLY

The content of the electronically submitted sequence listing in ASCII text file (Name: 725669_SA9-474PCCON_ST25.txt; Size: 67.8 KB; and Date of Creation: Jan. 14, 2022) is incorporated herein by reference in its entirety.

### BACKGROUND OF THE DISCLOSURE

Gene therapy offers the potential for a lasting means of treating a variety of diseases. Recent developments in gene therapy treatments employ the use of viral or non-viral vectors. There are several technologies currently being used for the production of gene therapy vectors.

For example, the baculovirus expression vector system (BEVS) is an established system for the production of gene therapy vectors. In this system, insect host cells are infected with a recombinant baculovirus. The insect host cells provide the necessary protein processing machinery in order to produce the product encoded by the recombinant baculovirus. BEVS has been used to produce product for many different applications, including vaccines, therapeutic proteins, protein cyrstollography, and product for basic and applied research. When producing viral vector for gene therapy, several baculovirus expression vectors are often used and infected into the insect host cells. The generation of each of the baculovirus expression vectors is time consuming, and drives up the cost of production.

Thus, there exists a need in the art for improved baculovirus expression vector systems, while avoiding the limitations of existing systems.

### SUMMARY OF THE DISCLOSURE

Provided herein is a baculovirus expression vector system comprising a baculovirus shuttle vector (bacmid) and/or stable cell lines engineered to produce a therapeutic drug product. Baculovirus expression vector systems provided herein comprise a recombinant bacmid comprising two or more foreign sequence insertion sites, and one or more donor vectors capable of mediating insertion of foreign sequence (e.g., a heterologous gene) into the foreign sequence insertion sites.

In one aspect, provided herein is a bacmid comprising: a bacterial replicon; a selectable marker sequence; a first reporter gene comprising a first preferential target site for the insertion of a transposon; a second reporter gene operably linked to a baculovirus-inducible promoter; and a second preferential target site capable of mediating a site-specific recombination event.

In certain exemplary embodiments, the bacterial replicon is a low-copy number replicon. In certain exemplary embodiments, the low-copy number replicon is a mini-F replicon.

In certain exemplary embodiments, the selectable marker sequence comprises an antibiotic resistance gene. In certain exemplary embodiments, the antibiotic resistance gene is a kanamycin resistance gene.

In certain exemplary embodiments, the first reporter gene encodes an enzyme capable of metabolizing a chromogenic substrate. In certain exemplary embodiments, the enzyme is LacZα or functional portion thereof. In certain exemplary embodiments, the chromogenic substrate is Blue-gal or X-gal.

In certain exemplary embodiments, the first preferential target site for the insertion of a transposon does not disrupt the reading frame of the first reporter gene. In certain exemplary embodiments, the first preferential target site is an attachment site for a bacterial transposon. In certain exemplary embodiments, the first preferential target site is an attachment site for a T7 transposon. In certain exemplary embodiments, the transposon is a T7 transposon.

In certain exemplary embodiments, the second reporter gene encodes a fluorescent protein. In certain exemplary embodiments, the fluorescent protein is a red fluorescent protein.

In certain exemplary embodiments, the baculovirus-inducible promoter is a 39K promoter.

In certain exemplary embodiments, second preferential target site comprises a LoxP site or a variant thereof.

In certain exemplary embodiments, the site-specific recombination event is mediated by a Cre recombinase.

In another aspect, provided herein is a bacmid comprising: a mini-F replicon; an antibiotic resistance gene; a LacZα gene or functional portion thereof comprising an attachment site for a T7 transposon; a gene encoding a fluorescent protein operably linked to a baculovirus-inducible promoter; and LoxP site or a variant thereof.

In another aspect, provided herein is a recombinant bacmid comprising: a bacterial replicon; a first selectable marker sequence; a heterologous sequence inserted into a first reporter gene, wherein the inserted heterologous sequence disrupts the reading frame of the first reporter gene; a second reporter gene operably linked to a baculovirus-inducible promoter; and a preferential target site capable of mediating a site-specific recombination event.

In certain exemplary embodiments, the heterologous sequence comprises a heterologous gene. In certain exemplary embodiments, the heterologous gene comprises a sequence encoding a protein.

In certain exemplary embodiments, the heterologous sequence further comprises an expression control sequence. In certain exemplary embodiments, the expression control sequence is operably linked to the sequence encoding a protein.

In certain exemplary embodiments, the expression control sequence comprises a baculovirus promoter. In certain exemplary embodiments, the baculovirus promoter is an immediate early, an early, a late, or a very late gene promoter. In certain exemplary embodiments, the baculovirus promoter is selected from the group consisting of a polyhedrin promoter, an immediate-early1 promoter, and an immediate-early2 promoter.

In certain exemplary embodiments, the expression control sequence comprises a polyadenylation signal.

In certain exemplary embodiments, the protein is a Rep protein isolated from a genome of a member of the viral family Parvoviridae. In certain exemplary embodiments, the protein is a parvoviral Rep protein. In certain exemplary embodiments, the parvoviral Rep protein is selected from the group consisting of a B19 Rep, an AAV2 Rep, a HBoV1 Rep, and a GPV Rep.

In certain exemplary embodiments, the heterologous sequence comprises a second selectable marker sequence. In certain exemplary embodiments, the second selectable marker sequence comprises a gentamycin resistance gene.

In certain exemplary embodiments, the bacterial replicon is mini-F replicon. In certain exemplary embodiments, the first selectable marker sequence comprises a kanamycin resistance gene. In certain exemplary embodiments, the first reporter gene encodes LacZα or functional portion thereof. In certain exemplary embodiments, the second reporter gene encodes a red fluorescent protein. In certain exemplary embodiments, wherein the baculovirus-inducible promoter is a 39K promoter. In certain exemplary embodiments, the second preferential target site comprises a LoxP site or a variant thereof. In certain exemplary embodiments, the site-specific recombination event is mediated by a Cre recombinase.

In another aspect, provided herein is a recombinant bacmid comprising: a heterologous sequence inserted into a mini-attTn7 site, wherein the heterologous sequence encodes a Rep, and wherein the inserted Rep disrupts the reading frame of the LacZα gene or functional portion thereof; and a LoxP site or a variant thereof.

In another aspect, provided herein is a recombinant bacmid comprising: a bacterial replicon; a first antibiotic resistance gene; a heterologous sequence inserted into a mini-attTn7 site, wherein the heterologous sequence encodes a Rep, and wherein the inserted Rep disrupts the reading frame of the LacZα gene or functional portion thereof; a gene encoding a fluorescent protein operably linked to a baculovirus-inducible promoter; and a LoxP site or a variant thereof.

In another aspect, provided herein is a recombinant bacmid comprising: a bacterial replicon; a first antibiotic resistance gene; a heterologous sequence inserted into a mini-attTn7 site, wherein the heterologous sequence encodes a B19 Rep, and wherein the inserted B19 Rep disrupts the reading frame of the LacZα gene or functional portion thereof; a gene encoding a fluorescent protein operably linked to a baculovirus-inducible promoter; and a LoxP site or a variant thereof.

In another aspect, provided herein is a recombinant bacmid comprising: a bacterial replicon; a first antibiotic resistance gene; a heterologous sequence inserted into a mini-attTn7 site, wherein the heterologous sequence encodes a GPV Rep, and wherein the inserted GPV Rep disrupts the reading frame of the LacZα gene or functional portion thereof; a gene encoding a fluorescent protein operably linked to a baculovirus-inducible promoter; and a LoxP site or a variant thereof.

In another aspect, provided herein is a recombinant bacmid comprising: a bacterial replicon; a first antibiotic resistance gene; a heterologous sequence inserted into a mini-attTn7 site, wherein the heterologous sequence encodes an AAV2 Rep, and wherein the inserted AAV2 Rep disrupts the reading frame of the LacZα gene or functional portion thereof; a gene encoding a fluorescent protein operably linked to a baculovirus-inducible promoter; and a LoxP site or a variant thereof.

In another aspect, provided herein is a nucleic acid vector comprising: a first origin of replication for propagating the nucleic acid vector in a first bacterial strain, wherein the first origin of replication is a conditional origin of replication; a second origin of replication for propagating the nucleic acid vector in a second bacterial strain; a multiple cloning site for the insertion of a heterologous sequence; a selectable marker sequence; a reporter gene; and a preferential target site capable of mediating a site-specific recombination event.

In certain exemplary embodiments, the first origin of replication is conditional upon the presence of a pi protein. In certain exemplary embodiments, the first origin of replication is R6Kγ.

In certain exemplary embodiments, the first bacterial strain comprises a pi protein.

In certain exemplary embodiments, the second origin of replication is pUC57.

In certain exemplary embodiments, the selectable marker sequence comprises an antibiotic resistance gene. In certain exemplary embodiments, the antibiotic resistance gene is an ampicillin resistance gene.

In certain exemplary embodiments, the reporter gene encodes a fluorescent protein. In certain exemplary embodiments, the fluorescent protein is a green fluorescent protein.

In certain exemplary embodiments, the preferential target site comprises a LoxP site or a variant thereof. In certain exemplary embodiments, the site-specific recombination event is mediated by a Cre recombinase.

In another aspect, provided herein is a nucleic acid vector comprising: a first origin of replication for propagating the nucleic acid vector in a first bacterial strain, wherein the first origin of replication is a conditional origin of replication; a second origin of replication for propagating the nucleic acid vector in a second bacterial strain; a multiple cloning site comprising a heterologous sequence; a selectable marker sequence; a reporter gene; and a preferential target site capable of mediating a site-specific recombination event.

In certain exemplary embodiments, the heterologous sequence comprises a heterologous gene. In certain exemplary embodiments, the heterologous gene comprises a sequence encoding a protein.

In certain exemplary embodiments, the heterologous sequence further comprises an expression control sequence. In certain exemplary embodiments, the expression control sequence is operably linked to the sequence encoding a protein. In certain exemplary embodiments, the expression control sequence comprises a tissue-specific promoter. In certain exemplary embodiments, the tissue-specific promoter is a tristetraprolin (TTP) or a murine transthyretin (mTTR) promoter. In certain exemplary embodiments, the expression control sequence comprises a polyadenylation signal. In certain exemplary embodiments, the polyadenylation signal is a bovine growth hormone polyadenylation signal. In certain exemplary embodiments, the expression control sequence comprises a post-transcriptional regulatory element. In certain exemplary embodiments, the post-transcriptional regulatory element is a woodchuck hepatitis virus post-transcriptional regulatory element (WPRE).

In certain exemplary embodiments, the protein is a therapeutic protein. In certain exemplary embodiments, the therapeutic protein is a clotting factor. In certain exemplary embodiments, the clotting factor is Factor VIII (FVIII). In certain exemplary embodiments, the clotting factor is FVIII-XTEN.

In certain exemplary embodiments, the heterologous sequence comprises a 5′ inverted terminal repeat (ITR). In certain exemplary embodiments, the heterologous sequence comprises a 3′ inverted terminal repeat (ITR). In certain exemplary embodiments, the 5′ ITR and the 3′ ITR are derived from a parvovirus. In certain exemplary embodiments, the parvovirus is selected from the group consisting of B19, GPV, HBoV1, and AAV2. In certain exemplary embodiments, the 5′ ITR is a wild-type or variant 5′ ITR derived from B19. In certain exemplary embodiments, the 5′ ITR is a wild-type or variant 5′ ITR derived from GPV. In certain exemplary embodiments, the 5′ ITR is a wild-type or variant 5′ ITR derived from AAV2. In certain exemplary embodiments, the 5′ ITR is a wild-type or variant 5′ ITR derived from HBoV1. In certain exemplary embodiments, the variant 5′ ITR is a truncated 5′ ITR. In certain exemplary embodiments, the 3′ ITR is a wild-type or variant 3′ ITR derived from B19. In certain exemplary embodiments, the 3′ ITR is a wild-type or variant 3′ ITR derived from GPV. In certain exemplary embodiments, the 3′ ITR is a wild-type or variant 3′ ITR derived from AAV2. In certain exemplary embodiments, the variant 3′ ITR is a truncated 3′ ITR. In certain exemplary embodiments, the 3′ ITR is a wild-type 3′ ITR derived from HBoV1.

In certain exemplary embodiments, the first origin of replication is conditional upon the presence of a pi protein. In certain exemplary embodiments, the first origin of replication is R6Kγ.

In certain exemplary embodiments, first bacterial strain comprises a pi protein.

In certain exemplary embodiments, the second origin of replication is pUC57.

In certain exemplary embodiments, the selectable marker sequence comprises an antibiotic resistance gene. In certain exemplary embodiments, the antibiotic resistance gene is an ampicillin resistance gene.

In certain exemplary embodiments, the reporter gene encodes a fluorescent protein.

In certain exemplary embodiments, the fluorescent protein is a green fluorescent protein.

In certain exemplary embodiments, the preferential target site comprises a LoxP site or a variant thereof. In certain exemplary embodiments, the site-specific recombination event is mediated by a Cre recombinase.

In another aspect, provided herein is a recombinant bacmid comprising: a first heterologous sequence inserted into a first reporter gene, wherein the inserted heterologous sequence disrupts the reading frame of the first reporter gene; a first preferential target site capable of mediating a site-specific recombination event; a multiple cloning site comprising a second heterologous sequence; and a second preferential target site capable of mediating a site-specific recombination event.

In certain exemplary embodiments, the first heterologous sequence comprises a first heterologous gene. In certain exemplary embodiments, the first heterologous sequence comprises an expression control sequence operably linked to a sequence encoding a protein.

In certain exemplary embodiments, the expression control sequence comprises a baculovirus promoter. In certain exemplary embodiments, the baculovirus promoter is an immediate early, an early, a late, or a very late promoter. In certain exemplary embodiments, the baculovirus promoter is selected from the group consisting of a polyhedrin promoter, an immediate-early1 promoter, and an immediate-early2 promoter.

In certain exemplary embodiments, the protein is a Rep protein isolated from a genome of a member of the viral family Parvoviridae. In certain exemplary embodiments, the protein is a parvoviral Rep protein. In certain exemplary embodiments, the parvoviral Rep protein is selected from the group consisting of a B19 Rep, an AAV2 Rep, a HBoV1 Rep, and a GPV Rep.

In certain exemplary embodiments, the second heterologous sequence comprises second a heterologous gene. In certain exemplary embodiments, the second heterologous sequence comprises a expression control sequence operably linked to a sequence encoding a protein. In certain exemplary embodiments, the expression control sequence comprises a tissue-specific promoter, a polyadenylation signal, and/or a post-transcriptional regulatory element. In certain exemplary embodiments, the tissue-specific promoter is a tristetraprolin (TTP) or a murine transthyretin (mTTR) promoter. In certain exemplary embodiments, the polyadenylation signal is a bovine growth hormone polyadenylation signal. In certain exemplary embodiments, the post-transcriptional regulatory element is a woodchuck hepatitis virus post-transcriptional regulatory element (WPRE).

In certain exemplary embodiments, the protein is a therapeutic protein. In certain exemplary embodiments, the therapeutic protein is a clotting factor. In certain exemplary embodiments, the clotting factor is Factor VIII (FVIII). In certain exemplary embodiments, the clotting factor is FVIII-XTEN.

In certain exemplary embodiments, the second heterologous sequence comprises a 5′ inverted terminal repeat (ITR). In certain exemplary embodiments, the second heterologous sequence comprises a 3′ inverted terminal repeat (ITR). In certain exemplary embodiments, the 5′ ITR is derived from a genome of a first member of the viral family Parvoviridae, and the 3′ ITR are derived from a genome of a second member of the viral family Parvoviridae. In certain exemplary embodiments, the first and the second members are the same. In certain exemplary embodiments, the first and the second members are different. In certain exemplary embodiments, the 5′ ITR and the 3′ ITR are derived from a parvovirus selected from the group consisting of B19, GPV, and AAV2. In certain exemplary embodiments, the 5′ ITR is a wild-type or truncated 5′ ITR derived from B19. In certain exemplary embodiments, the 5′ ITR is a wild-type or truncated 5′ ITR derived from GPV. In certain exemplary embodiments, the 5′ ITR is a wild-type or truncated 5′ ITR derived from AAV2. In certain exemplary embodiments, the 5′ ITR is a wild-type or truncated 5′ ITR derived from HBoV1. In certain exemplary embodiments, the 3′ ITR is a wild-type or truncated 3′ ITR derived from B19. In certain exemplary embodiments, the 3′ ITR is a wild-type or truncated 3′ ITR derived from GPV. In certain exemplary embodiments, the 3′ ITR is a wild-type or truncated 3′ ITR derived from AAV2. In certain exemplary embodiments, the 5′ ITR is a wild-type 3′ ITR derived from HBoV1.

In certain exemplary embodiments, the recombinant bacmid further comprises a bacterial replicon. In certain exemplary embodiments, the bacterial replicon is a mini-F replicon.

In certain exemplary embodiments, the recombinant bacmid further comprises one or more selectable marker sequences. In certain exemplary embodiments, the one or more selectable marker sequences comprise one or more antibiotic resistance genes. In certain exemplary embodiments, the one or more antibiotic resistance genes are selected from the group consisting of an ampicillin resistance gene, a kanamycin resistance gene, and a gentamycin resistance gene.

In certain exemplary embodiments, the first reporter gene encodes LacZα or functional portion thereof.

In certain exemplary embodiments, the recombinant bacmid further comprises at least a second and a third reporter gene. In certain exemplary embodiments, the second and the third reporter gene each encode a fluorescent protein. In certain exemplary embodiments, the fluorescent protein is a green fluorescent protein or a red fluorescent protein.

In certain exemplary embodiments, the first and the second preferential target site each comprise a LoxP site or a variant thereof. In certain exemplary embodiments, the site-specific recombination event is mediated by a Cre recombinase.

In another aspect, provided herein is a recombinant bacmid comprising: a sequence encoding a Rep inserted into a mini-attTn7 site, wherein the inserted Rep disrupts the reading frame of the LacZα gene or functional portion thereof; and a multiple cloning site comprising a heterologous sequence, wherein the heterologous sequence comprises from 5′ to 3′: a wild-type or truncated 5′ inverted terminal repeat derived from a first genome of a member of the viral family Parvoviridae; a sequence encoding a protein; one or more expression control sequences operably linked to the sequence encoding a protein; and a wild-type or truncated 3′ inverted terminal repeat derived from a second genome of a member of the viral family Parvoviridae.

In certain exemplary embodiments, the first and the second genome of the viral family Parvoviridae are the same. In certain exemplary embodiments, the first and the second genome of the viral family Parvoviridae are different.

In certain exemplary embodiments, the Rep is derived from the first or the second genome of the member of the viral family Parvoviridae.

In another aspect, provided herein is a recombinant bacmid comprising: a sequence encoding a B19 Rep inserted into a mini-attTn7 site, wherein the inserted B19 Rep disrupts the reading frame of the LacZα gene or functional portion thereof; and a multiple cloning site comprising a heterologous sequence, wherein the heterologous sequence comprises from 5′ to 3′: a wild-type or truncated 5′ inverted terminal repeat derived from B19; a sequence encoding a protein; one or more expression control sequences operably linked to the sequence encoding a protein; and a wild-type or truncated 3′ inverted terminal repeat derived from B19.

In another aspect, provided herein is a recombinant bacmid comprising: a sequence encoding a GPV Rep inserted into a mini-attTn7 site, wherein the inserted GPV Rep disrupts the reading frame of the LacZα gene or functional portion thereof; and a multiple cloning site comprising a heterologous sequence, wherein the heterologous sequence comprises from 5′ to 3′: a wild-type or truncated 5′ inverted terminal repeat derived from GPV; a sequence encoding a protein; one or more expression control sequences operably linked to the sequence encoding a protein; and a wild-type or truncated 3′ inverted terminal repeat derived from GPV.

In another aspect, provided herein is a recombinant bacmid comprising: a sequence encoding an AAV2 Rep inserted into a mini-attTn7 site, wherein the inserted AAV2 Rep disrupts the reading frame of the LacZα gene or functional portion thereof; and a multiple cloning site comprising a heterologous sequence, wherein the heterologous sequence comprises from 5′ to 3′: a wild-type or truncated 5′ inverted terminal repeat derived from AAV2; a sequence encoding a protein; one or more expression control sequences operably linked to the sequence encoding a protein; and a wild-type or truncated 3′ inverted terminal repeat derived from AAV2.

In another aspect, provided herein is a recombinant bacmid comprising: a sequence encoding an AAV2 HBoV1 Rep inserted into a mini-attTn7 site, wherein the inserted HBoV1 Rep disrupts the reading frame of the LacZα gene or functional portion thereof; and a multiple cloning site comprising a heterologous sequence, wherein the heterologous sequence comprises from 5′ to 3′: a wild-type 5′ inverted terminal repeat derived from HBoV1; a sequence encoding a protein; one or more expression control sequences operably linked to the sequence encoding a protein; and a wild-type 3′ inverted terminal repeat derived from HBoV1.

In certain exemplary embodiments, the protein is a therapeutic protein. In certain exemplary embodiments, the therapeutic protein is a clotting factor. In certain exemplary embodiments, the clotting factor is Factor VIII (FVIII). In certain exemplary embodiments, the clotting factor is FVIII-XTEN.

In another aspect, a host cell is provided comprising a bacmid as described herein. In another aspect, a host cell is provided comprising a recombinant bacmid as described herein. In another aspect, a host cell is provided comprising a vector as described herein.

In another aspect, provided herein is a method of generating a recombinant bacmid as described herein, comprising: introducing into a bacterial cell: a bacmid as described herein; and a donor nucleic acid molecule comprising a bacterial replicon operably linked to a transposon comprising a heterologous sequence as described herein; incubating the bacterial cell under conditions for transposition to occur; and isolating the recombinant bacmid from the bacterial cell.

In another aspect, provided herein is a method of generating a recombinant bacmid as described herein, comprising: introducing into a bacterial cell: a recombinant bacmid as described herein; a vector as described herein; and a Cre recombinase; incubating the bacterial cell under conditions for site-specific recombination to occur; and isolating the recombinant bacmid from the bacterial cell.

In another aspect, provided herein is a method of generating a recombinant baculovirus comprising transfecting a recombinant bacmid described herein into an insect cell under appropriate conditions.

In another aspect, provided herein is a method of generating a closed ended DNA (ceDNA) molecule comprising: transfecting a recombinant bacmid as described herein into an insect cell under appropriate conditions to generate a recombinant baculovirus; and infecting a second insect cell with the recombinant baculovirus under appropriate conditions to generate a ceDNA molecule.

In another aspect, provided herein is a method of generating a closed ended DNA (ceDNA) molecule comprising wild-type or truncated B19 inverted terminal repeats, comprising: transfecting a recombinant bacmid as described herein comprising wild-type or truncated B19 inverted terminal repeats into an insect cell under appropriate conditions to generate a recombinant baculovirus; and infecting a second insect cell with the recombinant baculovirus under appropriate conditions to generate a ceDNA molecule.

In another aspect, provided herein is a method of generating a closed ended DNA (ceDNA) molecule comprising wild-type or truncated GPV inverted terminal repeats, comprising: transfecting a recombinant bacmid as described herein comprising wild-type or truncated GPV inverted terminal repeats into an insect cell under appropriate conditions to generate a recombinant baculovirus; and infecting a second insect cell with the recombinant baculovirus under appropriate conditions to generate a ceDNA molecule.

In another aspect, provided herein is a method of generating a closed ended DNA (ceDNA) molecule comprising wild-type or truncated AAV2 inverted terminal repeats, comprising: transfecting a recombinant bacmid as described herein comprising wild-type or truncated AAV2 inverted terminal repeats into an insect cell under appropriate conditions to generate a recombinant baculovirus; and infecting a second insect cell with the recombinant baculovirus under appropriate conditions to generate a ceDNA molecule.

In another aspect, provided herein is a method of generating a closed ended DNA (ceDNA) molecule comprising wild-type HBoV1 inverted terminal repeats, comprising: transfecting a recombinant bacmid as described herein comprising wild-type HBoV1 inverted terminal repeats into an insect cell under appropriate conditions to generate a recombinant baculovirus; and infecting a second insect cell with the recombinant baculovirus under appropriate conditions to generate a ceDNA molecule.

In another aspect, provided herein is a plasmid comprising a nucleic acid sequence comprising from 5′ to 3′: a wild-type or truncated 5′ inverted terminal repeat derived from a genome of a first member of the viral family Parvoviridae; a sequence encoding a protein; one or more expression control sequences operably linked to the sequence encoding a protein; and a wild-type or truncated 3′ inverted terminal repeat derived from a genome of a second member of the viral family Parvoviridae.

In some embodiments, disclosed herein is a set of recombinant bacmids comprising a first bacmid and a second bacmid, wherein the first bacmid comprises a sequence encoding a Rep inserted into a mini-attTn7 site, wherein the inserted Rep disrupts the reading frame of a reporter gene or functional portion thereof; and wherein the second bacmid comprises a heterologous sequence, wherein the heterologous sequence comprises from 5′ to 3′: a wild-type or truncated 5′ inverted terminal repeat (ITR) derived from a first genome of a member of the viral family Parvoviridae; a sequence encoding a protein; one or more expression control sequences operably linked to the sequence encoding a protein; and a wild-type or truncated 3′ inverted terminal repeat (ITR) derived from a second genome of a member of the viral family Parvoviridae. In some embodiments, the 5′ ITR and the 3′ ITR are derived from a parvovirus selected from the group consisting of B19, GPV, HBoV1, and AAV2.

In certain exemplary embodiments, the one or more expression control sequences comprise a tissue-specific promoter, a polyadenylation signal, and/or a post-transcriptional regulatory element. In certain exemplary embodiments, the tissue-specific promoter is a tristetraprolin (TTP) or a murine transthyretin (mTTR) promoter. In certain exemplary embodiments, the polyadenylation signal is a bovine growth hormone polyadenylation signal. In certain exemplary embodiments, the post-transcriptional regulatory element is a woodchuck hepatitis virus post-transcriptional regulatory element (WPRE).

In certain exemplary embodiments, the protein is a therapeutic protein. In certain exemplary embodiments, the therapeutic protein is a clotting factor. In certain exemplary embodiments, the clotting factor is Factor VIII (FVIII). In certain exemplary embodiments, the clotting factor is FVIII-XTEN.

In certain exemplary embodiments, the first and/or the second members are selected from the group consisting of B19, GPV, HBoV1, and AAV2. In certain exemplary embodiments, the first and/or the second members are the same. In certain exemplary embodiments, the first and/or the second members are different.

In another aspect, provided herein is a stable cell line comprising a nucleic acid sequence as described herein, wherein the nucleic acid sequence is stably integrated in the genome of the stable cell line.

In certain exemplary embodiments, the stable cell line is a stable insect cell line. In certain exemplary embodiments, the stable insect cell line is Sf9.

In another aspect, provided herein is a method of generating a closed ended DNA (ceDNA) molecule comprising wild-type or truncated inverted terminal repeats, comprising introducing into a stable cell line as described herein a recombinant bacmid as described herein.

In another aspect, provided herein is a method of generating a closed ended DNA (ceDNA) molecule comprising wild-type or truncated inverted terminal repeats, comprising introducing into a host cell: a plasmid as described herein; and a recombinant bacmid as described herein.

In certain exemplary embodiments, the host cell is an insect cell. In certain exemplary embodiments, the insect cell is Sf9.

### BRIEF DESCRIPTION OF THE DRAWINGS

FIGS. 1A-1D are schematic representations of a recombinant bacmid according to one embodiment of the invention. FIG. 1A shows a schematic diagram of bMON14272 encoding kanamycin resistance marker (KanR), mini-attTn7 insertion site fused in-frame with the LacZα, and mini-F origin of replication (mini-F). FIG. 1B shows a schematic linear map of a synthetic transfer vector comprising a gene encoding Red Fluorescent Protein (RFP) under the AcMNPV 39K promoter (p39K) followed by the ets polyadenylation signal (etsPAS), LoxP recombination site, and EGT flanking gene sequences. FIG. 1C shows a schematic diagram of a recombinant bacmid (BIVVBac) encoding kanamycin resistance marker (KanR), mini-attTn7 insertion site fused in-frame with the LacZα, mini-F origin of replication (mini-F), a gene encoding Red Fluorescent Protein (RFP) gene under the AcMNPV 39K promoter (p39K) followed by the ets polyadenylation signal (etsPAS), and LoxP recombination site. FIG. 1D shows a schematic diagram of an E. coli strain (BIVVBacDH10B) harboring the BIVVBac bacmid and a Tn7 helper plasmid encoding transposase.

FIGS. 2A-2B are schematic representations of a Cre-LoxP donor vector according to one embodiment of the invention. FIG. 2A shows a schematic linear map of a synthetic DNA comprising a gene encoding enhanced GFP under the AcMNPV immediate-early1 (pIE1) promoter preceded by the AcMNPV transcriptional enhancer hr5 element and followed by the AcMNPV p10 polyadenylation signal (p10 PAS), LoxP recombination site (LoxP), ampicillin antibiotic resistance gene, multiple cloning site (MCS), ColE1, and R6Kγ origin of replications (Ori). FIG. 2B shows a schematic map of the Cre-LoxP donor vector according to one embodiment of the invention made by inserting the synthetic DNA (FIG. 2A) into a pUC57 vector.

FIGS. 3A-3F are schematic representations of Replication (Rep) protein expression constructs according to embodiments of the invention. FIG. 3A shows a schematic linear map of a synthetic DNA encoding B19 Rep gene under the AcMNPV polyhedrin or immediate-early1 (ie1) promoter followed by the SV40 polyadenylation signal (SV40 PAS). FIG. 3B shows a schematic map of a Tn7 transfer vector according to an embodiment of the invention made by inserting the B19.Rep synthetic DNA (FIG. 3A) into the pFastBac1 vector (Invitrogen). FIG. 3C shows a schematic linear map of a synthetic DNA encoding GPV Rep78 and Rep52 spliced genes under the AcMNPV polyhedrin promoter followed by the sv40 polyadenylation signal (SV40 PAS). Modified mRNA transcript (mRNA) is shown as wavy line with non-canonical start codon (CUG) of Rep78, canonical start codon (AUG) of Rep52, and stop codon (UAA). FIG. 3D shows a schematic map of a Tn7 transfer vector according to an embodiment of the invention made by inserting the GPV.Rep synthetic DNA (FIG. 3C) into the pFastBac1 vector (Invitrogen). FIG. 3E shows the schematic linear map of a synthetic DNA encoding AAV2 Rep78 and Rep52 spliced genes under the AcMNPV polyhedrin promoter followed by the sv40 polyadenylation signal (SV40 PAS). Modified mRNA transcript (mRNA) is shown as wavy line with non-canonical start codon (CUG) of Rep78, canonical start codon (AUG) of Rep52, and stop codon (UAA). FIG. 3F shows the schematic map of a Tn7 transfer vector according to an embodiment of the invention made by inserting the AAV2.Rep synthetic DNA (FIG. 3E) into the pFastBac1 vector (Invitrogen).

FIGS. 4A-4B are schematic representations of human FVIIIco6XTEN expression constructs. FIG. 4A shows schematic linear maps of expression constructs encoding codon-optimized human Factor VIII (FVIIIco6) comprising an XTEN 144 peptide (FVIIIco6XTEN) under the regulation of liver-specific TTPp promoter, the Woodchuck Posttranscriptional Regulatory Element (WPRE), and the Bovine Growth Hormone Polyadenylation (bGHpA) signal. The hFVIIIco6XTEN expression cassette is flanked by the symmetric truncated (a), symmetric wildtype (WT), and asymmetric (Asy) ITRs of B19 (Δ135, WT), GPV (Δ162, WT, and Asy), or AAV2 (WT, Asy1, Asy2, Asy3), respectively. ITR sequences as indicated can be found in Table 1. FIG. 4B shows schematic linear maps of Cre-LoxP donor vectors encoding hFVIIIco6XTEN expression cassette flanked by the ITRs of B19, GPV, or AAV2 as described in FIG. 4A cloned into the Cre-LoxP donor vector as described in FIG. 2B.

FIGS. 5A-5G show schematics of recombinant baculovirus expression vectors (BEVs) comprising sequence encoding Rep, and confirmation studies of the same. FIG. 5A is an agarose gel electrophoresis image of restriction enzyme mapping of recombinant BIVVBac bacmids encoding B19.Rep, GPV.Rep, or AAV2.Rep (BIVVBac.IE1.B19.Rep^{Tn7}, BIVVBac.Polh.GPV.Rep^{Tn7}, and BIVVBac.Polh.AAV2.Rep^{Tn7}, respectively) in comparison with the parental BIVVBac and bMON14272 bacmids. FIG. 5B shows a schematic map of AcBIVVBac.IE1.B19.Rep^{Tn7}. FIG. 5C shows a schematic map of AcBIVVBac.Polh.GPV.Rep^{Tn7}. FIG. 5D shows a schematic map of AcBIVVBac.Polh.AAV2.Rep^{Tn7}. FIG. 5E shows B19.REP immunoblotting in the plaque purified clones (clones 1-6; Cl #1, Cl #2, Cl #3, Cl #4, Cl #5, Cl #6) of recombinant AcBIVVBac.IE1.B19.Rep^{Tn7} BEV with polyclonal anti-B19 NS1 (1:2500) as primary antibody and IRDye@680RD Donkey anti-Rabbit LI-COR (1:10,000) as secondary antibody. Precision Plus Protein™ Dual Color (Bio-Rad) was used as a standard and is shown on the left. FIG. 5F shows GPV.REP immunoblotting in the plaque purified clones (clones 1-6; Cl #1, Cl #2, Cl #3, Cl #4, Cl #5, Cl #6) of recombinant AcBIVVBac.Polh.GPV.Rep^{Tn7} BEV with polyclonal anti-GPV REP peptide antibody (1:500) as primary antibody and IRDye@680RD Donkey anti-Rabbit LI-COR (1:10,000) as secondary antibody. Precision Plus Protein™ Dual Color (Bio-Rad) was used as a standard and is shown in the left. FIG. 5G shows AAV2.REP immunoblotting in the plaque purified clones of recombinant AcBIVVBac.Polh.AAV2.Rep^{Tn7} BEV with monoclonal anti-AAV2 Rep (1:500) as primary antibody and IRDye@800CW Donkey anti-Mouse LI-COR (1:10,000) as secondary antibody. Precision Plus Protein™ Dual Color (Bio-Rad) was used as a standard and is shown on the left.

FIGS. 6A-6C are schematic representations of BEVs comprising sequences encoding Rep and hFVIIIco6XTEN. FIG. 6A shows a schematic map of recombinant BEV encoding B19.Rep expression cassette and hFVIIIco6XTEN expression cassette flanked by B19 ITRs as indicated (AcBIVVBac(B19.Rep)FVII.B19.ITRs^{LoxP}). FIG. 6B shows a schematic map of recombinant BEV encoding GPV.Rep expression cassette and hFVIIIco6XTEN expression cassette flanked by GPV ITRs as indicated (AcBIVVBac(GPV.Rep)FVIII.GPV.ITRs^{LoxP}). FIG. 6C shows a schematic map of recombinant BEV encoding AAV2.Rep expression cassette and hFVIIIco6XTEN expression cassette flanked by AAV2 ITRs (AcBIVVBac(AAV2.Rep)FVIII.AAV2.ITRs^{LoxP}). ITR sequences as indicated in FIGS. 6A-6C can be found in Table 1.

FIGS. 7A-7C show the production of human FVIIIco6XTEN ceDNA vector using a baculovirus expression vector system according to one embodiment of the invention. FIG. 7A is a schematic map of the recombinant BEV encoding GPV.Rep expression cassette and hFVIIIco6XTEN expression cassette flanked by the GPV asymmetric ITRs (AcBIVVBac(GPV.Rep)FVIII.GPV.Asy.ITRs^{LoxP}). ITR sequences as indicated can be found in Table 1. FIG. 7B is an agarose gel electrophoresis image of ceDNA vector isolated from Sf9 cells infected with plaque purified clones of AcBIVVBac(GPV.Rep)FVIII.GPV.Asy.ITRs^{LoxP} BEV (clones 1-6; Cl #1, Cl #2, Cl #3, Cl #4, Cl #5, Cl #6). The DNA band corresponding to the size of hFVIIIco6XTEN is indicated by arrows. FIG. 7C is an agarose gel electrophoresis image of ceDNA vector obtained from recombinant AcBIVVBac(GPV.Rep)FVIII.GPV.Asy.ITRs^{LoxP} BEV Clone #4 (as shown in FIG. 7B) loaded at different volumes. The DNA band corresponding to the size of hFVIIIco6XTEN is indicated by an arrow.

FIGS. 8A-8B show the materials used in generating stable human FVIIIco6XTEN encoding insect cell lines. FIG. 8A shows a schematic map of a plasmid encoding neomycin resistance marker under the AcMNPV immediate early (ie1) promoter preceded by the transcriptional enhancer hr5 element and followed by the AcMNPV p10 polyadenylation signal. FIG. 8B shows schematic maps of the hFVIIIco6XTEN expression cassettes flanked by the B19, GPV, or AAV2 ITRs, which are stably integrated into the Sf9 cell genome to generate stable cell lines. A list of the stable cell lines generated is shown in Table 5, and sequences of the ITRs are shown in Table 1.

FIGS. 9A-9C are agarose gel electrophoresis images showing the production of human FVIIIco6XTEN ceDNA vector from stable cell lines. FIG. 9A is an agarose gel electrophoresis image of ceDNA vector isolated from Sf cell line-1 and -2, encoding hFVIIIco6XTEN, flanked by the symmetric truncated (B19Δ135) or symmetric wildtype (B19.WT) ITRs, respectively. FIG. 9B is an agarose gel electrophoresis image of ceDNA vector isolated from Sf cell line-3, -4, and -5, encoding hFVIIIco6XTEN, flanked by the symmetric truncated (GPVΔ162), symmetric wildtype (GPV.WT), or asymmetric (GPV.Asy) ITRs, respectively. FIG. 9C is an agarose gel electrophoresis image of ceDNA vector isolated from Sf cell line-6, -7, -8, and -9, encoding hFVIIIco6XTEN, flanked by the symmetric wildtype (AAV2.WT), asymmetric1 (AAV2.Asy1), asymmetric2 (AAV2.Asy2), or asymmetric3 (AAV2.Asy3), respectively. The stable cell lines used in FIGS. 9A-9C are shown in Table 5, and sequences of the ITRs are shown in Table 1.

FIG. 10 is a plot showing hFVIII activity measured by the Chromogenix Coatest® SP Factor VIII chromogenic assay. ceDNAs isolated from stable cell lines were transfected in Huh7 cells, and cell-free supernatant was harvested 48 and 72 h post transfection and tested for hFVIII detection by the chromogenic assay. Solid lines represent the ceDNA samples and dotted lines represent the plasmid DNA samples used as controls.

FIGS. 11A-11F are schematic representations of Replication (Rep) protein expression constructs according to embodiments of the invention. FIG. 11A shows a schematic linear map of a synthetic DNA encoding B19 Rep gene under the OpMNPV immediate-early (OpIE2) promoter followed by the sv40 polyadenylation signal (SV40 PAS). FIG. 11B shows a schematic map of the transient expression plasmid made by replacing the IE1 promoter with OpIE2 in pFastBac.IE1.B19.Rep construct as shown in FIG. 3B. FIG. 11C shows a schematic linear map of a synthetic DNA encoding GPV Rep78 and Rep52 spliced genes under the OpMNPV immediate-early (OpIE2) promoter followed by the sv40 polyadenylation signal (SV40 PAS). Modified mRNA transcript (mRNA) is shown as wavy line with non-canonical start codon (CUG) of Rep78, canonical start codon (AUG) of Rep52, and stop codon (UAA). FIG. 11D shows a schematic map of the transient expression plasmid made by replacing the polyhedrin promoter with OpIE2 in pFastBac.Polh.GPV.Rep construct as shown in FIG. 3D. FIG. 11E shows the schematic linear map of a synthetic DNA encoding AAV2 Rep78 and Rep52 spliced genes under the OpMNPV immediate-early (OpIE2) promoter followed by the sv40 polyadenylation signal (SV40 PAS). Modified mRNA transcript (mRNA) is shown as wavy line with non-canonical start codon (CUG) of Rep78, canonical start codon (AUG) of Rep52, and stop codon (UAA). FIG. 11F shows the schematic map of the transient expression plasmid made by replacing the polyhedrin promoter with OpIE2 in pFastBac.Polh.AAV2.Rep construct as shown in FIG. 3F.

FIGS. 12A-12C are schematic representation of modified FVIIIXTEN expression cassettes with parvoviral ITRs according to embodiments of the invention, the liver-specific modified mouse transthyretin (mTTR) promoter (mTTR482), and the (V2.0) codon-optimized BDDco-FVIIIXTEN transgene (V2.0 FVIIIXTEN) (SEQ ID NO: 19). FIG. 12A shows schematic linear map of modified FVIIIXTEN expression cassette flanked by the AAV2 WT ITRs (SEQ ID NO: 5, SEQ ID NO: 6). FIG. 12B shows schematic linear map of modified FVIIIXTEN expression cassette flanked by the B19 WT (SEQ ID NO: 2) or B19 Minimal (SEQ ID NO: 16). FIG. 12C shows schematic linear map of modified FVIIIXTEN expression cassette flanked by the GPVΔ120 (SEQ ID NO: 17) or GPVΔ186 (SEQ ID NO: 18).

FIGS. 13A-13C are schematic representation of approaches used for ceDNA production in the baculovirus system according to one embodiment of the invention. FIG. 13A shows a schematic diagram of One BAC approach, where a single recombinant BEV encoding V2.0 FVIIIXTEN and Rep genes at different loci was used for infection in Sf9 cells for ceDNA production. FIG. 13B shows a schematic diagram of Two BAC approach, where Sf9 cells were co-infected with recombinant BEVs encoding V2.0 FVIIIXTEN and/or Rep genes for ceDNA production. FIG. 13C shows a schematic diagram of a stable cell line approach, where the FVIIIXTEN expression cassette was stably integrated into the Sf9 cell genome and was rescued by infecting recombinant BEV encoding Rep gene for ceDNA production.

FIGS. 14A-14C shows the production of human FVIIIXTEN ceDNA vector from AAV2 One BAC according to one embodiment of the invention. FIG. 14A is a schematic diagram of One BAC approach of FVIIIXTEN ceDNA vector production in Sf9 cells using recombinant BEV encoding AAV2 Rep gene under the AcMNPV polyhedrin promoter and human FVIIIXTEN expression cassette comprising the V2.0 FVIIIXTEN and flanked by the AAV2 WT ITRs (AcBIVVBac(mTTR.FVIIIXTEN.AAV2.WT.ITRs)Polh.AAV2.Rep^{LoxP}). FIG. 14B shows the schematic map of AcBIVVBac(mTTR.FVIIIXTEN.AAV2.WT.ITRs)Polh.AAV2.Rep^{LoxP} BEV. FIG. 14C is an agarose gel electrophoresis image of ceDNA vector isolated from Sf9 cells infected with titrated virus stock (P2) of AcBIVVBac(mTTR.FVIIIXTEN.AAV2.WT.ITRs) Polh.AAV2.Rep^{LoxP} BEV. The DNA bands corresponding to the size of FVIIIXTEN ceDNA (ceDNA), baculoviral DNA (vDNA) and Sf9 cell genomic DNA (gDNA) are indicated by arrows.

FIGS. 15A-15C shows the production of human FVIIIXTEN ceDNA vector from B19 One BAC according to one embodiment of the invention. FIG. 15A is a schematic diagram of One BAC approach of FVIIIXTEN ceDNA vector production in Sf9 cells using recombinant BEV encoding B19 NS1 gene under the AcMNPV polyhedrin promoter and human FVIIIXTEN expression cassette comprising the V2.0 FVIIIXTEN and flanked by the B19 WT ITRs (AcBIVVBac(mTTR.FVIIIXTEN.B19.WT.ITRs)Polh.B19.NS1^{LoxP}) FIG. 15B shows the schematic map of AcBIVVBac(mTTR.FVIIIXTEN.B19.WT.ITRs)Polh.B19.NS1^{LoxP} BEV. FIG. 15C is an agarose gel electrophoresis image of ceDNA vector isolated from Sf9 cells infected with titrated virus stock (P2) of AcBIVVBac(mTTR.FVIIIXTEN.B19.WT.ITRs)Polh.B19.NS1^{LoxP} BEV. The DNA bands corresponding to the size of FVIIIXTEN ceDNA (ceDNA), baculoviral DNA (vDNA) and Sf9 cell genomic DNA (gDNA) are indicated by arrows.

FIGS. 16A-16C shows the production of human FVIIIXTEN ceDNA vector from AAV2 Two BACs according to one embodiment of the invention. FIG. 16A is a schematic diagram of Two BAC approach of FVIIIXTEN ceDNA vector production, where Sf9 cells are co-infected with a recombinant BEVs encoding FVIIIXTEN expression cassette comprising the V2.0 FVIIIXTEN and flanked by the AAV2 WT ITRs (AcBIVVBac.mTTR.FVIIIXTEN.AAV2.WT.ITRs^{Tn7}) and/or encoding AAV2 Rep gene under the AcMNPV polyhedrin promoter (AcBIVVBac.Polh.AAV2.Rep^{Tn7}). FIG. 16B shows the schematic maps of AcBIVVBac.mTTR.FVIIIXTEN.AAV2.WT.ITRs^{Tn7} and AcBIVVBac.Polh.AAV2.Rep^{Tn7} BEVs. FIG. 16C is an agarose gel electrophoresis image of ceDNA vector isolated from Sf9 cells co-infected at different MOIs of AcBIVVBac.mTTR.FVIIIXTEN.AAV2.WT.ITRs^{Tn7} and AcBIVVBac.Polh.AAV2.Rep^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 17A-17C shows the production of human FVIIIXTEN ceDNA vector from B19 Two BACs according to one embodiment of the invention. FIG. 17A is a schematic diagram of Two BAC approach of FVIIIXTEN ceDNA vector production, where Sf9 cells are co-infected with a recombinant BEVs encoding FVIIIXTEN expression cassette comprising the V2.0 FVIIIXTEN and flanked by the B19 WT ITRs (AcBIVVBac.mTTR.FVIIIXTEN.B19.WT.ITRs^{Tn7}) and/or encoding B19 NS1 gene under the AcMNPV polyhedrin promoter (AcBIVVBac.Polh.B19.NS1^{Tn7}). FIG. 17B shows the schematic maps of AcBIVVBac.mTTR.FVIIIXTEN.B19.WT.ITRs^{Tn7} and AcBIVVBac.Polh.B19.NS1^{Tn7} BEVs. FIG. 17C is an agarose gel electrophoresis image of ceDNA vector isolated from Sf9 cells co-infected at different MOIs of AcBIVVBac.mTTR.FVIIIXTEN.B19.WT.ITRs^{Tn7} and AcBIVVBac.Polh.B19.NS1^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 18A-18C shows the production of human FVIIIXTEN ceDNA vector from GPV Two BACs according to one embodiment of the invention. FIG. 18A is a schematic diagram of Two BAC approach of FVIIIXTEN ceDNA vector production, where Sf9 cells are co-infected with a recombinant BEVs encoding FVIIIXTEN expression cassette comprising the V2.0 FVIIIXTEN and flanked by the GPVΔ120 ITRs (AcBIVVBac.mTTR.FVIIIXTEN.GPVΔ120.ITRs^{Tn7}) and/or encoding GPV NS1 gene under the AcMNPV polyhedrin promoter (AcBIVVBac.Polh.GPV.NS1^{Tn7}). FIG. 18B shows the schematic maps of AcBIVVBac.mTTR.FVIIIXTEN.GPVΔ120.ITRs^{Tn7} and AcBIVVBac.Polh.GPV.NS1^{Tn7} BEVs. FIG. 18C is an agarose gel electrophoresis image of ceDNA vector isolated from Sf9 cells co-infected at different MOIs of AcBIVVBac.mTTR.FVIIIXTEN.GPVΔ120.ITRs^{Tn7} and AcBIVVBac.Polh.GPV.NS1^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 19A-19C shows the production of human FVIIIXTEN ceDNA vector from AAV2 Stable Cell Line according to one embodiment of the invention. FIG. 19A shows a schematic diagram of Stable Cell Line approach of FVIIIXTEN ceDNA vector production, where Stable Cell Line encoding FVIIIXTEN expression cassette comprising the V2.0 FVIIIXTEN and flanked by the AAV2 WT ITRs is infected with a recombinant BEV encoding AAV2 Rep gene under the AcMNPV polyhedrin promoter (AcBIVVBac.Polh.AAV2.Rep^{Tn7}). FIG. 19B shows the schematic map of AcBIVVBac.Polh.AAV2.Rep^{Tn7} BEV. FIG. 19C is an agarose gel electrophoresis image of ceDNA vector isolated from AAV2 Stable Cell Line infected at different MOIs of AcBIVVBac.Polh.AAV2.Rep^{Tn7} BEV as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 20A-20C shows the production of human FVIIIXTEN ceDNA vector from B19 Stable Cell Line according to one embodiment of the invention. FIG. 20A shows a schematic diagram of Stable Cell Line approach of FVIIIXTEN ceDNA vector production, where Stable Cell Line encoding FVIIIXTEN expression cassette comprising the V2.0 FVIIIXTEN and flanked by the B19 Minimal ITRs is infected with a recombinant BEV encoding B19 NS1 gene under the AcMNPV polyhedrin promoter (AcBIVVBac.Polh.B19.NS1^{Tn7}). FIG. 20B shows the schematic map of AcBIVVBac.Polh.B19.NS1^{Tn7} BEV. FIG. 20C is an agarose gel electrophoresis image of ceDNA vector isolated from B19 Stable Cell Line infected at different MOIs of AcBIVVBac.Polh.B19.NS1^{Tn7} BEV as indicated. The DNA bands corresponding to the size of FVIIIXTEN ceDNA vector (ceDNA), baculoviral DNA (vDNA) and Sf9 cell genomic DNA (gDNA) are indicated by arrows.

FIGS. 21A-21C shows the production of human FVIIIXTEN ceDNA vector from GPV Stable Cell Line according to one embodiment of the invention. FIG. 21A shows a schematic diagram of Stable Cell Line approach of FVIIIXTEN ceDNA vector production, where Stable Cell Line encoding FVIIIXTEN expression cassette comprising the V2.0 FVIIIXTEN and flanked by the GPVΔ120 ITRs is infected with a recombinant BEV encoding GPV NS1 gene under the AcMNPV polyhedrin promoter (AcBIVVBac.Polh.GPV.NS1^{Tn7}). FIG. 21B shows the schematic map of AcBIVVBac.Polh.GPV.NS1^{Tn7} BEV. FIG. 21C is an agarose gel electrophoresis image of ceDNA vector isolated from GPV Stable Cell Line infected at different MOIs of AcBIVVBac.Polh.GPV.NS1^{Tn7} BEV as indicated. The DNA bands corresponding to the size of FVIIIXTEN ceDNA vector (ceDNA), baculoviral DNA (vDNA) and Sf9 cell genomic DNA (gDNA) are indicated by arrows.

FIG. 22A-22E shows the workflow of FVIIIXTEN ceDNA vector production and purification using Two BAC approach according to one embodiment of the invention. FIG. 22A shows a schematic of Sf9 cell expansion and duration (Day 0-2), where the cells are sequentially scaled up from small scale (0.5 L) to large scale culture (1.5 L) flasks to achieve the cell density of 2.5 to 3.0×10⁶/mL in serum-free ESF921 medium. FIG. 22B shows a schematic of Sf9 large culture (1.5 L) flask infection and duration of incubation (Day 2-6), where the cells are co-infected with a recombinant BEV encoding a recombinant BEV encoding FVIIIXTEN expression cassette comprising the V2.0 FVIIIXTEN and flanked by the HBoV1 ITRs (AcBIVVBac.mTTR.FVIIIXTEN.HBoV1.ITRs^{Tn7}) and/or encoding HBoV1 NS1 gene under the AcMNPV polyhedrin promoter (AcBIVVBac.Polh.HBoV1.NS1^{Tn7}) at an MOI of 0.1 and 0.01 plaque-forming units (pfu)/cell, respectively. FIG. 22C shows an image of Plasmid Giga Prep Purification kit and agarose gel electrophoresis with duration of processing (Day 6-7), where the cell density and viability of infected cells are measured daily, and the cells were pelleted by low-speed centrifugation once the cell viability reached at 70-80%. The FVIIIXTEN ceDNA vector was purified from infected cell pellets by the PureLink™ HiPure Expi Plasmid Gigaprep Kit (Invitrogen) and an aliquot was ran on agarose gel electrophoresis to determine the productivity of FVIIIXTEN ceDNA (ceDNA), baculoviral DNA (vDNA) and/or Sf9 cell genomic DNA (gDNA). FIG. 22D shows an image of Bio-Rad Model 491 Prep Cell and agarose gel electrophoresis with duration of processing (Day 7-12), where the Giga-prep purified DNAs were loaded onto Preparative agarose gel in Prep Cell for separating the FVIIIXTEN ceDNA (˜8.5 kb fragment) from the high molecular weight DNAs. Elution fractions collected at 70-80 min intervals from the Preparative Agarose Gel Electrophoresis were analyzed on 0.8 to 1.2% agarose gel to determine the purity of FVIIIXTEN ceDNA. FIG. 22E shows an image of agarose gel electrophoresis, where the fractions collected from the Prep Cell were combined and precipitated with 1/10th vol of 3M NaOAc (pH 5.5) and 3 vol of 100% ethanol to obtain purified FVIIIXTEN ceDNA. The gel image shows the purity of FVIIIXTEN ceDNA in comparison with the starting material with arrows indicating DNA bands corresponding to the size of FVIIIXTEN ceDNA vector (ceDNA), baculoviral DNA (vDNA) and Sf9 cell genomic DNA (gDNA).

FIG. 23 shows the graphical representation of plasma FVIII activity levels measured by the Chromogenix Coatest® SP Factor VIII chromogenic assays. FIG. 23 shows the graphical plot of plasma FVIII activity levels measured in blood samples collected at different intervals from hFVIIIR593C^{+/+}/HemA mice systemically injected via hydrodynamic tail-vein injection with 80, 40, or 12 μg/kg of FVIIIXTEN HBoV1 (wtHBoV1) or AAV2 (wtAAV2) ITRs ceDNAs.

FIGS. 24A-24C shows the production of human FVIIIXTEN ceDNA vector using One BAC approach according to one embodiment of the invention. FIG. 24A is a schematic diagram of One BAC approach of FVIIIXTEN ceDNA vector production in Sf9 cells using recombinant BEV encoding HBoV1 NS1 gene under the AcMNPV polyhedrin promoter and human FVIIIXTEN expression cassette flanked by the HBoV1 ITRs (AcBIVVBac(mTTR.FVIIIXTEN.HBoV1.ITRs)Polh.HBoV1.NS1^{LoxP}). FIG. 24B shows the schematic map of AcBIVVBac(mTTR.FVIIIXTEN.HBoV1.ITRs)Polh.HBoV1.NS1^{LoxP} BEV FIG. 24C is an agarose gel electrophoresis image of ceDNA vector isolated from Sf9 cells infected with titrated virus stock (P2) of (AcBIVVBac(mTTR.FVIIIXTEN.HBoV1.ITRs)Polh.HBoV1.NS1^{LoxP})BEV. The DNA bands corresponding to the size of FVIIIXTEN ceDNA (ceDNA), baculoviral DNA (vDNA) and Sf9 cell genomic DNA (gDNA) are indicated by arrows.

FIGS. 25A-25C shows the production of human FVIIIXTEN ceDNA vector using Two BAC approach according to one embodiment of the invention. FIG. 25A is a schematic diagram of Two BAC approach of FVIIIXTEN ceDNA vector production, where Sf9 cells are co-infected with a recombinant BEVs encoding FVIIIXTEN expression cassette flanked by the HBoV1 ITRs (AcBIVVBac.mTTR.FVIIIXTEN.HBoV1.ITRs^{Tn7}) and/or encoding HBoV1 NS1 gene under the AcMNPV polyhedrin promoter (AcBIVVBac.Polh.HBoV1.NS1^{Tn7}). FIG. 25B shows the schematic maps of AcBIVVBac.mTTR.FVIIIXTEN.HBoV1.ITRs^{Tn7} and AcBIVVBac.Polh.HBoV1.NS1^{Tn7} BEVs. FIG. 25C is an agarose gel electrophoresis images of ceDNA vector isolated from Sf9 cells co-infected at different MOIs of constant ratio or different ratios of constant MOI of AcBIVVBac.mTTR.FVIIIXTEN.HBoV1.ITRs^{Tn7} and AcBIVVBac.Polh.HBoV1.NS1^{Tn7} BEVs as indicated. The DNA bands corresponding to the size of FVIIIXTEN ceDNA vector (ceDNA), baculoviral DNA (vDNA) and Sf9 cell genomic DNA (gDNA) are indicated by arrows.

### DETAILED DESCRIPTION

Provided herein is a baculovirus expression vector system suitable for the expression of two or more foreign sequences. Also provided are methods of using a baculovirus expression vector system described herein, e.g., to produce ceDNA.

### I. Definitions

It is to be noted that the term “a” or “an” entity refers to one or more of that entity: for example, “a nucleotide sequence” is understood to represent one or more nucleotide sequences. Similarly, “a therapeutic protein” and “a miRNA” is understood to represent one or more therapeutic protein and one or more miRNA, respectively. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Also as used herein, “and/or” refers to and encompasses any and all possible combinations of one or more of the associated listed items, as well as the lack of combinations when interpreted in the alternative (“or”).

“Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop.

In the present disclosure, at least one ITR is an ITR of a non-adenovirus associated virus (non-AAV). In certain embodiments, the ITR is an ITR of a non-AAV member of the viral family Parvoviridae. In some embodiments, the ITR is an ITR of a non-AAV member of the genus Dependovirus or the genus Erythrovirus. In particular embodiments, the ITR is an ITR of a goose parvovirus (GPV), a Muscovy duck parvovirus (MDPV), or an erythrovirus parvovirus B19 (also known as parvovirus B19—also referred to herein as “B19”, primate erythroparvovirus 1, B19 virus, and erythrovirus). In certain embodiments, one ITR of two ITRs is an ITR of an AAV. In other embodiments, one ITR of two ITRs in the construct is an ITR of an AAV serotype selected from serotype 1, 2, 3, 4, 5, 6, 7, 8, 9, 10, 11 and any combination thereof. In one particular embodiment, the ITR is derived from AAV serotype 2, e.g., an ITR of AAV serotype 2.

In certain aspects of the present disclosure, the nucleic acid molecule comprises two ITRs, a 5′ ITR and a 3′ ITR, wherein the 5′ ITR is located at the 5′ terminus of the nucleic acid molecule, and the 3′ ITR is located at the 3′ terminus of the nucleic acid molecule. The 5′ ITR and the 3′ ITR can be derived from the same virus or different viruses. In certain embodiments, the 5′ ITR is derived from an AAV and the 3′ ITR is not derived from an AAV virus (e.g., a non-AAV). In some embodiments, the 3′ ITR is derived from an AAV and the 5′ ITR is not derived from an AAV virus (e.g., a non-AAV). In other embodiments, the 5′ ITR is not derived from an AAV virus (e.g., a non-AAV), and the 3′ ITR is derived from the same or a different non-AAV virus.

The term “parvovirus” as used herein encompasses the family Parvoviridae, including but not limited to autonomously-replicating parvoviruses and Dependoviruses. The autonomous parvoviruses include, for example, members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

Exemplary autonomous parvoviruses include, but are not limited to, porcine parvovirus, mice minute virus, canine parvovirus, mink entertitus virus, bovine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al. VIROLOGY, volume 2, chapter 69 (4th ed., Lippincott-Raven Publishers).

The term “non-AAV” as used herein encompasses nucleic acids, proteins, and viruses from the family Parvoviridae excluding any adeno-associated viruses (AAV) of the Parvoviridae family. “Non-AAV” includes but is not limited to autonomously-replicating members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers).

The term “derived from,” as used herein, refers to a component that is isolated from or made using a specified molecule or organism, or information (e.g., amino acid or nucleic acid sequence) from the specified molecule or organism. For example, a nucleic acid sequence (e.g., ITR) that is derived from a second nucleic acid sequence (e.g., ITR) can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some embodiments, a nucleotide or amino acid sequence that is derived from a second nucleotide or amino acid sequence has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the second nucleotide or amino acid sequence, respectively, wherein the first nucleotide or amino acid sequence retains the biological activity of the second nucleotide or amino acid sequence. In other embodiments, an ITR derived from an ITR of a non-AAV (or AAV) is at least 90% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 80% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 70% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 60% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 50% identical to the non-AAV ITR (or AAV ITR, respectively), wherein the non-AAV (or AAV) ITR retains a functional property of the non-AAV ITR (or AAV ITR, respectively).

In certain embodiments, an ITR derived from an ITR of a non-AAV (or AAV) comprises or consists of a fragment of the ITR of the non-AAV (or AAV). In some embodiments, the ITR derived from an ITR of a non-AAV (or AAV) comprises or consists of a fragment of the ITR of the non-AAV (or AAV), wherein the fragment comprises at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 125 nucleotides, at least about 150 nucleotides, at least about 175 nucleotides, at least about 200 nucleotides, at least about 225 nucleotides, at least about 250 nucleotides, at least about 275 nucleotides, at least about 300 nucleotides, at least about 325 nucleotides, at least about 350 nucleotides, at least about 375 nucleotides, at least about 400 nucleotides, at least about 425 nucleotides, at least about 450 nucleotides, at least about 475 nucleotides, at least about 500 nucleotides, at least about 525 nucleotides, at least about 550 nucleotides, at least about 575 nucleotides, or at least about 600 nucleotides; wherein the ITR derived from an ITR of a non-AAV (or AAV) retains a functional property of the non-AAV ITR (or AAV ITR, respectively). In certain embodiments, the ITR derived from an ITR of a non-AAV (or AAV) comprises or consists of a fragment of the ITR of the non-AAV (or AAV), wherein the fragment comprises at least about 129 nucleotides, and wherein the ITR derived from an ITR of a non-AAV (or AAV) retains a functional property of the non-AAV ITR (or AAV ITR, respectively). In certain embodiments, the ITR derived from an ITR of a non-AAV (or AAV) comprises or consists of a fragment of the ITR of the non-AAV (or AAV), wherein the fragment comprises at least about 102 nucleotides, and wherein the ITR derived from an ITR of a non-AAV (or AAV) retains a functional property of the non-AAV ITR (or AAV ITR, respectively).

In some embodiments, the ITR derived from an ITR of a non-AAV (or AAV) comprises or consists of a fragment of the ITR of the non-AAV (or AAV), wherein the fragment comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the length of the ITR of the non-AAV (or AAV).

In certain embodiments, a nucleotide or amino acid sequence that is derived from a second nucleotide or amino acid sequence has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to a homologous portion of the second nucleotide or amino acid sequence, respectively, when properly aligned, wherein the first nucleotide or amino acid sequence retains the biological activity of the second nucleotide or amino acid sequence. In other embodiments, an ITR derived from an ITR of a non-AAV (or AAV) is at least 90% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 80% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 70% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 60% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 50% identical to a homologous portion of the non-AAV ITR (or AAV ITR, respectively), when properly aligned, wherein the first nucleotide or amino acid sequence retains the biological activity of the second nucleotide or amino acid sequence.

A “capsid-free” or “capsid-less” vector or nucleic acid molecule refers to a vector construct free from a capsid. In some embodiments, the capsid-less vector or nucleic acid molecule does not contain sequences encoding, e.g., an AAV Rep protein. In some embodiments, a capsid-free or capsid-less vector can comprise covalently closed ends, referred to herein as a “closed-ended DNA (ceDNA).” In general, ceDNA described herein can be produced using a baculovirus expression system of the invention, and the ceDNA generally comprise at least one nucleic acid that encodes a heterologous gene product that is flanked on either side by ITRs (e.g., AAV or non-AAV ITRs).

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “genetic cassette” or “expression cassette” means a DNA sequence capable of directing expression of a particular polynucleotide sequence in an appropriate host cell, comprising a promoter operably linked to a polynucleotide sequence of interest. A genetic cassette may encompass nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence. In some embodiments, the genetic cassette comprises a polynucleotide which encodes a gene product. In some embodiments, the genetic cassette comprises a polynucleotide which encodes a miRNA. In some embodiments, the genetic cassette comprises a heterologous polynucleotide sequence.

A polynucleotide which encodes a product, e.g., a miRNA or a gene product (e.g., a polypeptide such as a therapeutic protein), can include a promoter and/or other expression (e.g., transcription or translation) control sequences operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control sequences, besides a promoter, for example enhancers, operators, repressors, and transcription termination signals, can also be operably associated with a coding region to direct gene product expression.

“Expression control sequences” refer to regulatory nucleotide sequences, such as promoters, enhancers, terminators, and the like, that provide for the expression of a coding sequence in a host cell. Expression control sequences generally encompass any regulatory nucleotide sequence which facilitates the efficient transcription and translation of the coding nucleic acid to which it is operably linked. Non-limiting examples of expression control sequences include include promoters, enhancers, translation leader sequences, introns, polyadenylation recognition sequences, RNA processing sites, effector binding sites, or stem-loop structures. A variety of expression control sequences are known to those skilled in the art. These include, without limitation, expression control sequences which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other expression control sequences include those derived from vertebrate genes such as actin, heat shock protein, bovine growth hormone and rabbit β-globin, as well as other sequences capable of controlling gene expression in eukaryotic cells. Additional suitable expression control sequences include tissue-specific promoters and enhancers as well as lymphokine-inducible promoters (e.g., promoters inducible by interferons or interleukins). Other expression control sequences include intronic sequences, post-transcriptional regulatory elements, and polyadenylation signals. Additional exemplary expression control sequences are discussed elsewhere in the present disclosure.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In some embodiments, the nucleic acid molecule comprises a tissue specific promoter. In certain embodiments, the tissue specific promoter drives expression of the therapeutic protein, e.g., the clotting factor, in the liver, e.g., in hepatocytes and/or endothelial cells. In particular embodiments, the promoter is selected from the group consisting of a mouse transthyretin promoter (mTTR), a native human factor VIII promoter, a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, a phosphoglycerate kinase (PGK) promoter and any combination thereof. In some embodiments, the promoter is selected from a liver specific promoter (e.g., α1-antitrypsin (AAT)), a muscle specific promoter (e.g., muscle creatine kinase (MCK), myosin heavy chain alpha (aMHC), myoglobin (MB), and desmin (DES)), a synthetic promoter (e.g., SPc5-12, 2R5Sc5-12, dMCK, and tMCK) and any combination thereof. In one particular embodiment, the promoter comprises a TTP promoter.

The terms “restriction endonuclease” and “restriction enzyme” are used interchangeably and refer to an enzyme that binds and cuts within a specific nucleotide sequence within double stranded DNA.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construct, which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Culture,” “to culture” and “culturing,” as used herein, means to incubate cells under in vitro conditions that allow for cell growth or division or to maintain cells in a living state. “Cultured cells,” as used herein, means cells that are propagated in vitro.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, Wis.), the GCG suite of programs (Wisconsin Package Version 9.0, Genetics Computer Group (GCG), Madison, Wis.), BLASTP, BLASTN, BLASTX (Altschul et al., J. Mol. Biol. 215:403 (1990)), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

As used herein, nucleotides corresponding to nucleotides in a particular sequence of the disclosure are identified by alignment of the sequence of the disclosure to maximize the identity to a reference sequence. The number used to identify an equivalent amino acid in a reference sequence is based on the number used to identify the corresponding amino acid in the sequence of the disclosure.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of the therapeutic protein, e.g., the clotting factor, e.g., FVIII. In another embodiment, the heterologous nucleotide sequence encodes a polypeptide that increases the hydrodynamic radius of the therapeutic protein, e.g., the clotting factor, e.g., FVIII. In other embodiments, the heterologous nucleotide sequence encodes a polypeptide that improves one or more pharmacokinetic properties of the therapeutic protein without significantly affecting its biological activity or function (e.g., a procoagulant activity). In some embodiments, the therapeutic protein is linked or connected to the polypeptide encoded by the heterologous nucleotide sequence by a linker. Non-limiting examples of polypeptide moieties encoded by heterologous nucleotide sequences include an immunoglobulin constant region or a portion thereof, albumin or a fragment thereof, an albumin-binding moiety, a transferrin, the PAS polypeptides of U.S. Pat Application No. 20100292130, a HAP sequence, transferrin or a fragment thereof, the C-terminal peptide (CTP) of the β subunit of human chorionic gonadotropin, albumin-binding small molecule, an XTEN sequence, FcRn binding moieties (e.g., complete Fc regions or portions thereof which bind to FcRn), single chain Fc regions (ScFc regions, e.g., as described in US 2008/0260738, WO 2008/012543, or WO 2008/1439545), polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others, or two or more combinations thereof. In some embodiments, the polypeptide encoded by the heterologous nucleotide sequence is linked to a non-polypeptide moiety. Non-limiting examples of the non-polypeptide moieties include polyethylene glycol (PEG), albumin-binding small molecules, polysialic acid, hydroxyethyl starch (HES), a derivative thereof, or any combinations thereof.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, and two or more combinations thereof.

As used herein, the term “bacmid,” refers to a shuttle vector that can be propagated in both E. coli and insect cells. A recombinant bacmid refers to a bacmid comprising a heterologous sequence (e.g., a heterologous sequence encoding a heterologous gene).

### II. Baculovirus Expression Vector System

Provided herein is a baculovirus expression vector system comprising a baculovirus shuttle vector (bacmid) and/or stable cell lines engineered to produce a therapeutic drug product. Baculovirus expression vector systems provided herein comprise a recombinant bacmid comprising two or more foreign sequence insertion sites, and one or more donor vectors capable of mediating insertion of foreign sequence (e.g., a heterologous gene) into the foreign sequence insertion sites.

In some embodiments, the therapeutic drug product is a protein. In some embodiments, the therapeutic drug product is a nucleic acid. In some embodiments, the therapeutic drug product is a recombinant protein that may find use in, e.g., various applications including vaccines, protein replacement therapy (e.g., enzyme replacement therapy), and recombinant proteins for basic and applied research. In certain embodiments, the therapeutic drug product is a DNA therapeutic drug substance that is a plasmid-like, capsid free, nucleic acid molecule that encodes a target sequence. The DNA therapeutic drug substance may be in the form of a covalently closed-end DNA (ceDNA). Generally, ceDNA comprises a therapeutic protein-coding gene positioned between a first inverted terminal repeat (5′ ITR) and a second ITR (3′ ITR).

In certain embodiments, the 5′ ITR and the 3′ ITR are adeno-associated virus (AAV) ITRs or non-AAV ITRs. In certain embodiments, non-AAV ITRs are ITRs obtained from a member of the viral family Parvoviridae. Suitable ITR sequences include AAV ITRs of AAV serotypes known to those of skill in the art. Suitable AAV and non-AAV ITR sequences are described in PCT Publication Nos. WO2019032898A1, WO2020033863A1, and WO2017152149A1, the disclosures of which are herein incorporated by reference in their entireties. For example, non-AAV ITR sequences may be derived from Goose parvovirus (GPV) or parvovirus B19 (also referred to herein as “B19”).

### A. Baculovirus Shuttle Vector (Bacmid)

Baculoviruses are the most prominent viruses that infect insects. Over 500 baculovirus isolates have been identified, the majority of which originated in insects of the order Lepidoptera. The two most common isolates are Autographa californica multiple nucleopolyhedrovirus (AcMNPV) and Bombyx mori nucleopolyhedrovirus (BmNPV). When producing viral or non-viral vector for gene therapy, several baculovirus expression vectors are often required to be infected into insect host cells. The generation of each of the baculovirus expression vectors is time consuming, and drives up the cost of production, representing a significant disadvantage of baculovirus expression vector systems prior to the present invention.

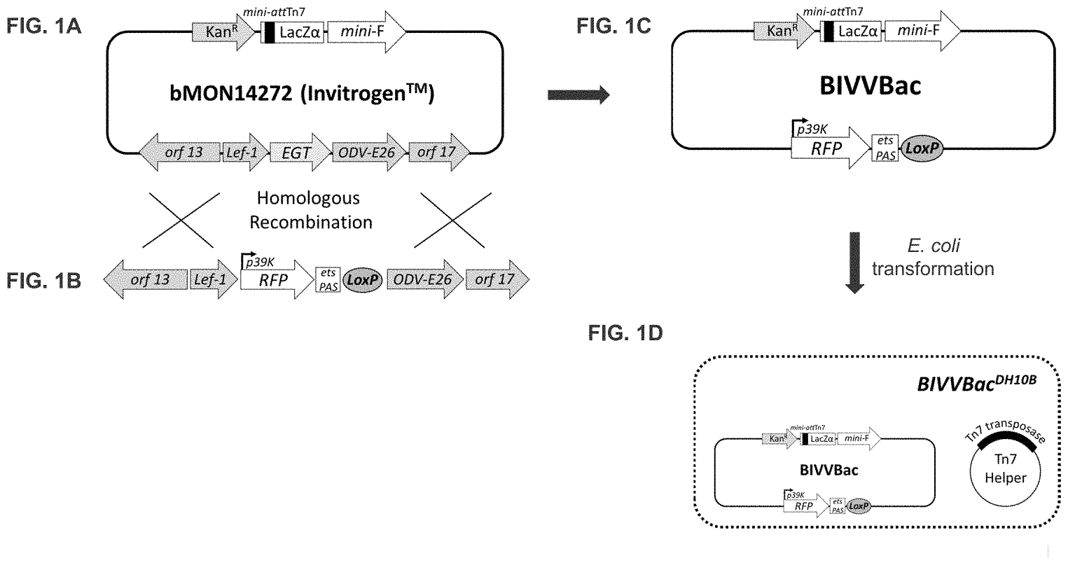

In certain embodiments, a baculovirus expression vector system provided herein comprises a recombinant bacmid, referred to herein as “BIVVBac.” A representative schematic of BIVVBac is shown in FIG. 1C. In certain embodiments, the BIVVBac is a genetically modified AcMNPV that comprises at least two foreign sequence insertion sites. A baculovirus expression vector system comprising a bacmid that comprises at least two foreign sequence insertion sites allows for the reduction in total number of baculovirus expression vectors that need to be generated. In some embodiments, a single baculovirus expression vector of the present invention is required to produce a gene therapy vector.

In certain embodiments, the BIVVBac comprises a first and a second foreign sequence insertion site. The first and the second foreign sequence insertion sites may be different, utilizing different machinery to drive insertion of a foreign sequence (e.g., heterologous sequence, heterologous gene). Insertion of a foreign sequence may be driven by any method known in the art. For example, a foreign sequence may be inserted by transposition or site-specific recombination. A foreign sequence insertion site may be designed to be comprised within a reporter gene such that upon insertion of the foreign sequence, the reporter gene becomes disrupted. Disruption of the reporter gene may aid in the identification of bacmid clones having a foreign sequence inserted therein. In such embodiments, the foreign sequence insertion site is fused in-frame with the reporter gene, or the reporter gene is fused in-frame with the foreign sequence insertion site.

In certain embodiments, the first and the second foreign sequence insertion sites are located in different loci within AcMNPV. In certain embodiments, the first and the second foreign sequence insertion sites are located in different non-essential loci within AcMNPV. Various non-essential loci of AcMNPV are known to those of skill in the art. For example, the polyhedrin gene and the EGT gene are non-essential AcMNPV genes for viral replication in insect cells. Accordingly, in certain embodiments, the first foreign sequence insertion site is located in the polyhedrin locus of AcMNPV, and the second foreign sequence insertion site is located in the EGT locus of AcMNPV.

In certain embodiments, the first foreign sequence insertion site allows for the insertion of a foreign sequence via transposition. In certain embodiments, the first foreign sequence insertion site comprises a preferential target site for the insertion of a transposon. In certain embodiments, the first foreign sequence insertion site is a preferential target site for the insertion of a transposon. In certain embodiments, the first foreign sequence insertion site is a preferential target site that is an attachment site for a bacterial transposon. Suitable bacterial transposons and their corresponding attachment sites are known to those of skill in the art. For example, the transposon Tn7 is known for its ability to transpose to a specific site of a bacterial chromosome (attTn7) at a high frequency. Accordingly, in certain embodiments, the first foreign sequence insertion site is a preferential target site that is an attachment site for a Tn7 transposon (e.g., attTn7). In some embodiments, the first foreign sequence insertion site is a preferential target site that is an attachment site for a mini-Tn7 transposon (e.g., mini-attTn7, the minimal DNA sequence required for recognition by Tn7 transposition factors and insertion of a Tn7 transposon).

In certain embodiments, the first foreign sequence insertion site is fused in-frame with a reporter gene. The reporter gene can be any reporter gene known in the art, including, e.g., luciferase (Luc), green fluorescent protein (GFP), red fluorescent protein (RFP), a fluorescent protein of a specific color, chloramphenicol acetyltransferase (CAT), β-galactosidase (LacZ), β-glucuronidase (Gus), and the like. In certain embodiments, the first foreign sequence insertion site is fused in-frame with a reporter gene that encodes an enzyme capable of metabolizing a chromogenic substrate. The enzyme capable of metabolizing a chromogenic substrate can be any enzyme known in the art having the same property, for example, LacZ or a functional portion thereof. Reporter genes encoding enzymes capable of metabolizing a chromogenic substrate find use in color-based screening of positive insertion events. For example, LacZ finds use in blue-white screening, where a functional LacZ gene product cleaves the chromogenic substrate X-gal or Blue-gal resulting in the production of a blue pigment. In blue-white screening, presence of a functional LacZ by alpha complementation means that the insertion event failed, and blue colonies represent clones in which the foreign sequence did not get inserted. On the other hand, white colonies represent clones in which the foreign sequence inserted successfully, thereby disrupting the production of functional LacZ gene product and preventing cleavage of the chromogenic substrate (e.g., X-gal).

Accordingly, in certain embodiments, the first foreign sequence insertion site is a preferential target site for a bacterial transposon that is fused in-frame with a sequence encoding LacZα or a functional portion thereof. In certain embodiments, the first foreign sequence insertion site is a preferential target site that is an attachment site for a Tn7 transposon (e.g., attTn7) fused in-frame with a sequence encoding LacZα or a functional portion thereof. Upon successful transposition, the transposon disrupts the sequence encoding LacZα, resulting in disrupted production of functional LacZ gene product.

In certain embodiments, the second foreign sequence insertion site allows for the insertion of a foreign sequence via site-specific recombination. In certain embodiments, the second foreign sequence insertion site comprises a preferential target site capable of mediating a site-specific recombination event. Various site-specific recombinase technologies are known to those of skill in the art. For example, the Cre-loxP system mediates site-specific recombination via Cre recombinase which is capable of recognizing 34 base pair DNA sequences called loxP sites. Accordingly, the second foreign sequence insertion site is a preferential target site for Cre mediated recombination. In certain embodiments, the second foreign sequence insertion site is a preferential target site comprising a loxP site or a variant thereof capable of being recognized by Cre recombinase.

A recombinant bacmid of the present invention comprises other elements required for its ability to be propagated in both bacterial (e.g., E. coli) and insect cells. For example, a recombinant bacmid of the present invention comprises a bacterial replicon. In certain embodiments, the bacmid comprises a bacterial replicon. Various bacterial replicons are known to those of skill in the art, and include, for example, replicons of F plasmid derived origin. In certain embodiments, a suitable bacterial replicon is the mini-F replicon, which is a derivative of the F plasmid comprised of DNA regions oriS and incC required for replication and regulation. In certain embodiments, the bacterial replicon is a low-copy number replicon. In certain embodiments, the low-copy number replicon is a mini-F replicon.

Other elements of the recombinant bacmid of the present invention includes one or more selectable marker sequences, and other reporter genes. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. In certain embodiments, the recombinant bacmid comprises a selectable marker sequence comprising an antibiotic resistance gene. In certain embodiments, the antibiotic resistance gene is a kanamycin resistance gene and confers resistance to kanamycin. Examples of reporters known and used in the art include: luciferase (Luc), green fluorescent protein (GFP), chloramphenicol acetyltransferase (CAT), β-galactosidase (LacZ), 3-glucuronidase (Gus), and the like. In some cases, selectable markers can also be considered to be reporters. In certain embodiments, the recombinant bacmid comprises a reporter gene encoding a fluorescent protein. In certain embodiments, the fluorescent protein is a red fluorescent protein.

Those of skill in the art will recognize that the various elements of the recombinant bacmid described herein are in operable linkage with each other. Each of the various coding sequences in the bacmid may be in operable linkage with a regulatory region comprising, e.g., a promoter sequence. Any promoter sequence known in the art may be suitable.

Accordingly, a recombinant bacmid of the present invention comprises a mini-F replicon, an antibiotic resistance gene, a LacZα or functional portion thereof comprising an attachment site for a Tn7 transposon, a gene encoding a fluorescent protein operably linked to a baculovirus-inducible promoter, and a LoxP site or a variant thereof.

In certain exemplary embodiments, the recombinant bacmid of the present invention is BIVVBac as shown in FIG. 1C. In certain embodiments, BIVVBac encodes the AcMNPV C6 genome and has been engineered to encode two foreign sequence insertion sites: 1) a mini-attTn7 in the Polyhedrin locus, and 2) a LoxP in the EGT locus. The mini-attTn7 insertion sequence is fused in-frame with a Lac-promoter-driven E. coli LacZα fragment for the X-gal-mediated blue/white screening of recombinant bacmid following Tn7-mediated transposition, and the LoxP site for Cre-mediated in-vitro or in-vivo recombination. BIVVBac also comprises a red fluorescent protein gene under the control of the AcMNPV 39K promoter and is followed by the ets polyadenylation signal.

In certain exemplary embodiments, the BIVVBac is introduced into a bacterial strain capable of allowing Tn7 mediated transposition. In certain embodiments, the bacterial strain is an E. coli strain. In certain embodiments, the bacterial strain is E. coli DH10B bacteria. In certain embodiments, a helper plasmid encoding the Tn7 transposase genes tnsA-E and tetracycline resistance is introduced into DH10B E. coli harboring BIVVBac. DH10B E. coli harboring BIVVBac and the helper plasmid is referred to herein as BIVVBac^{DH10B}.

Any foreign sequence (e.g., heterologous sequence) can be introduced into BIVVBac via Tn7 mediated transposition. In certain embodiments, a foreign sequence is introduced into BIVVBac via Tn7 mediated transposition by introducing a Tn7 transfer vector comprising the foreign sequence into BIVVBac^{DH10B}, resulting in a BIVVBac comprising the foreign sequence. In certain embodiments, the BIVVBac comprising the foreign sequence comprises a bacterial replicon, a first selectable marker sequence, a foreign sequence (e.g., a heterologous sequence) inserted into a first reporter gene, wherein the inserted foreign sequence disrupts the reading frame of the first reporter gene, a second reporter gene operably linked to a baculovirus-inducible promoter, and a preferential target site capable of mediating a site-specific recombination event.

In certain exemplary embodiments, for the purposes of producing a gene therapy vector, AAV or non-AAV Rep genes are introduced into BIVVBac via Tn7 mediated transposition. In certain embodiments, a Tn7 transfer vector comprising an AAV or non-AAV Rep gene is introduced into BIVVBac^{DH10B}, resulting in a BIVVBac comprising a Rep gene. In certain embodiments, the recombinant bacmid comprises: a mini-F replicon; a first antibiotic resistance gene; a sequence encoding a B19 Rep inserted into a LacZα or functional portion thereof, wherein the inserted B19 Rep disrupts the reading frame of the LacZα or functional portion thereof; a gene encoding a fluorescent protein operably linked to a baculovirus-inducible promoter; and a LoxP site or a variant thereof. In certain embodiments, the recombinant bacmid comprises: a mini-F replicon; a first antibiotic resistance gene; a sequence encoding a GPV Rep inserted into a LacZα or functional portion thereof, wherein the inserted GPV Rep disrupts the reading frame of the LacZα or functional portion thereof; a gene encoding a fluorescent protein operably linked to a baculovirus-inducible promoter; and a LoxP site or a variant thereof. In certain embodiments, the recombinant bacmid comprises: a mini-F replicon; a first antibiotic resistance gene; a sequence encoding a AAV2 Rep inserted into a LacZα or functional portion thereof, wherein the inserted AAV2 Rep disrupts the reading frame of the LacZα or functional portion thereof; a gene encoding a fluorescent protein operably linked to a baculovirus-inducible promoter; and a LoxP site or a variant thereof.

Any foreign sequence (e.g., heterologous sequence) can be introduced into BIVVBac via Cre mediated recombination. In certain embodiments, a foreign sequence is introduced into BIVVBac via Cre mediated recombination, wherein the foreign sequence resides on a Cre-LoxP transfer vector described herein. Methods of Cre mediated recombination is well known to those of skill in the art.

In certain exemplary embodiments, for the purposes of producing a gene therapy vector, a therapeutic protein-coding gene flanked by symmetric or assymetric AAV or non-AAV inverted terminal repeats (ITRs) is introduced into BIVVBac via Cre mediated recombination. In certain embodiments, the recombinant bacmid comprises: a mini-F replicon; an antibiotic resistance gene; a LacZα or functional portion thereof comprising an attachment site for a T7 transposon; a first preferential target site capable of mediating a site-specific recombination event (e.g., a first LoxP site); a therapeutic protein-coding gene flanked by symmetric or assymetric AAV or non-AAV ITRs; and a second preferential target site capable of mediating a site-specific recombination event (e.g., a second LoxP site).

Accordingly, for the purposes of producing a gene therapy vector, the recombinant bacmid comprises: a sequence encoding a B19 Rep inserted into a LacZα or functional portion thereof, wherein the inserted B19 Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from parvovirus B19; a sequence encoding a protein; one or more expression control sequences operably linked to the sequence encoding a protein; and a wild-type or truncated 3′ inverted terminal repeat derived from parvovirus B19.

Accordingly, for the purposes of producing a gene therapy vector, the recombinant bacmid comprises: a sequence encoding a GPV Rep inserted into a LacZα or functional portion thereof, wherein the inserted GPV Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from GPV; a sequence encoding a protein; one or more expression control sequences operably linked to the sequence encoding a protein; and a wild-type or truncated 3′ inverted terminal repeat derived from GPV.

Accordingly, for the purposes of producing a gene therapy vector, the recombinant bacmid comprises: a sequence encoding a AAV2 Rep inserted into a LacZα or functional portion thereof, wherein the inserted AAV2 Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from AAV2; a sequence encoding a protein; one or more expression control sequences operably linked to the sequence encoding a protein; and a wild-type or truncated 3′ inverted terminal repeat derived from AAV2.

Accordingly, for the purposes of producing a gene therapy vector, the recombinant bacmid comprises: a sequence encoding a human bocavirus (HBoV1) Rep inserted into a mini-attTn7 site, LacZα, or functional portion thereof, wherein the inserted HBoV1 Rep disrupts the reading frame of the LacZα or functional portion thereof; and a multiple cloning site comprising a heterologous sequence, wherein the heterologous sequence comprises from 5′ to 3′: a wild-type 5′ inverted terminal repeat derived from HBoV1; a sequence encoding a protein; one or more expression control sequences operably linked to the sequence encoding a protein; and a wild-type 3′ inverted terminal repeat derived from HBoV1.

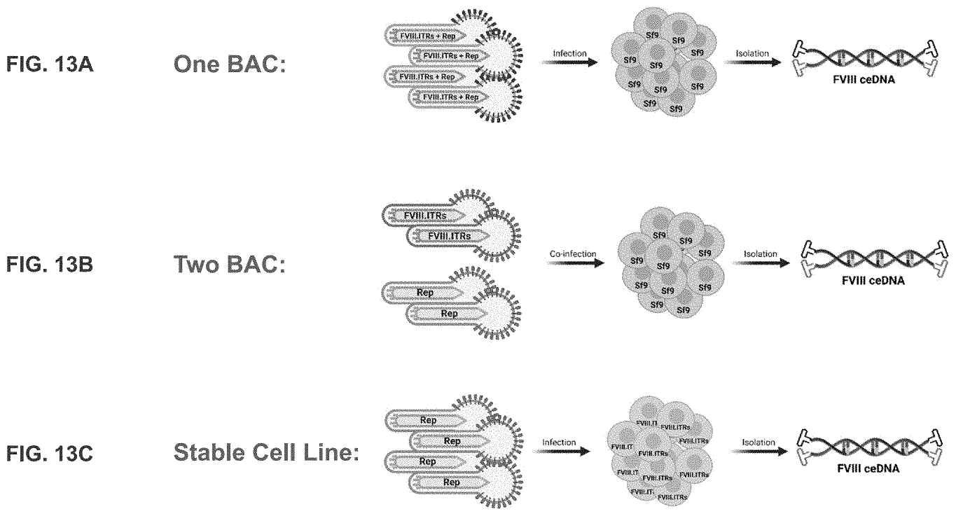

In certain embodiments, the BIVVBac bacmid is used to produce closed-end DNA (ceDNA). In some embodiments, ceDNA is produced using a single baculovirus expression vector. In this “OneBAC” approach, a single baculovirus expression vector (e.g., BIVVBac) encodes all essential elements required for ceDNA production in the baculovirus system and could potentially be used in any baculovirus permissive cell lines for ceDNA production. This approach is depicted in FIG. 13A.

In certain embodiments, ceDNA is produced using multiple multiple baculovirus expression vectors. In this “TwoBAC” approach, essential elements required for ceDNA production are inserted into two different baculoviruses (e.g., two BIVVBac bacmids) and could potentially be used for co-infection in any cell lines permissive for baculovirus infection. This approach is depicted in FIG. 13B.

In certain embodiments, ceDNA is produced by a stable cell line. In this approach, the essential elements required for ceDNA production are inserted in both components of the baculovirus system. This approach is depicted in FIG. 13C. A stable cell line can be generated by stably integrating a protein encoding sequence under the control of a baculovirus gene promoter (e.g., a baculovirus constitutive gene promoter). In certain embodiments, the stable cell line is a stable insect cell line.

### B. Transfer Vectors

The recombinant bacmid described herein comprises at least two foreign sequence insertion sites. In certain embodiments, the at least two foreign sequence insertion sites are 1) a mini-attTn7 for Tn7-mediated transposition, and 2) a LoxP for Cre-mediated site-specific recombination. Accordingly, a baculovirus expression vector system of the present invention further comprises two or more transfer vectors that comprise the foreign sequence to be inserted into the foreign sequence insertion sites of the recombinant bacmid.

### Tn7 Transfer Vector

In certain embodiments, a transfer vector comprises a foreign sequence to be inserted into the mini-attTn7 of the recombinant bacmid. In certain embodiments, the transfer vector is a Tn7 transfer vector. In certain embodiments, the transfer vector is a mini-Tn7 transfer vector. In certain embodiments, the Tn7 transfer vector comprises the left and right ends of Tn7 (Tn7L and Tn7R) capable of mediating transposition of the foreign sequence into the mini-attTn7 of the recombinant bacmid.

In certain exemplary embodiments, for the purposes of producing a gene therapy vector, the Tn7 transfer vector comprises the coding sequence for AAV or non-AAV Rep. In some embodiments, the Rep expression constructs are provided, wherein the Rep gene is of a non-AAV or AAV cloned under the regulation of baculovirus gene promoter in the pFastBac1 transfer vector (Invitrogen). In certain embodiments, the baculovirus gene promoter is the promoter for immediate-early (ie1). In certain embodiments, the baculovirus gene promoter is the promoter for the polyhedrin gene. Suitable baculovirus gene promoters are known to those of skill in the art. In certain embodiments, a suitable baculovirus gene promoter is an immediate early, an early, a late, or a very late gene promoter

In certain embodiments, the Rep coding sequence is modified such that the canonical start codon of Rep78 is modified to a non-canonical start codon. In certain embodiments, other AUG codons before the start codon of Rep52 are mutated to bear either a silent mutation (in the case of out-of-frame) or to encode a conservative amino acid substitution (in the case of in-frame) to allow the expression of Rep78 and Rep52 polypeptides from a single mRNA transcript through a leaky ribosomal scanning mechanism.

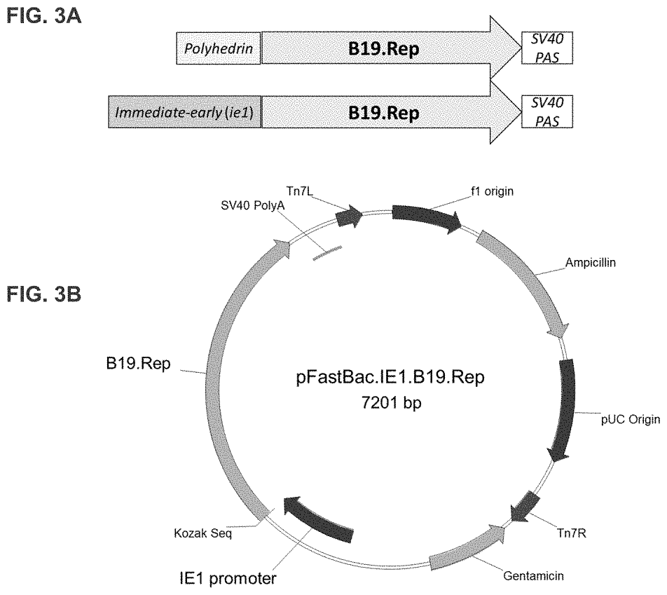

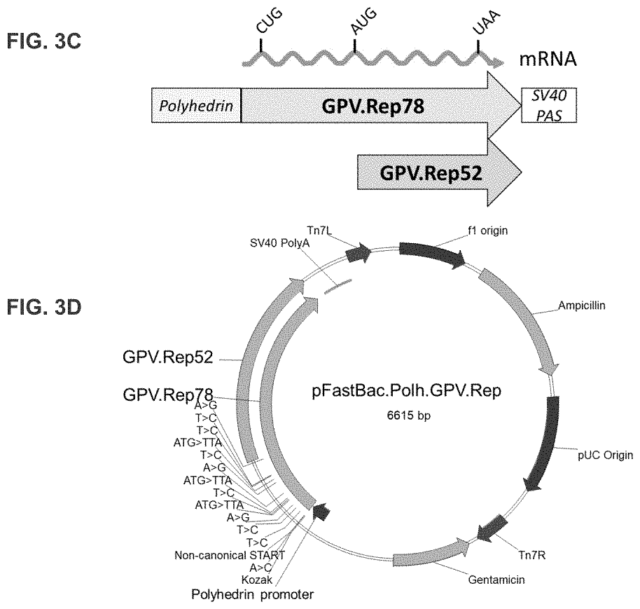

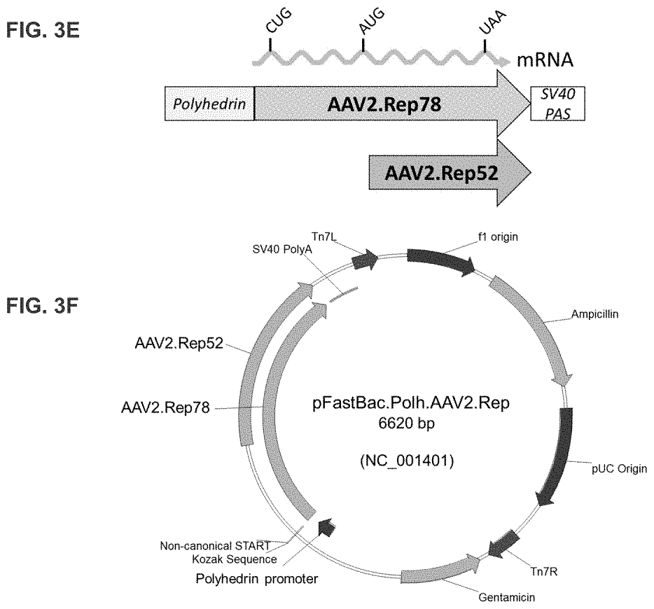

In certain embodiments, the Tn7 transfer vector comprises the left and right ends of Tn7 (Tn7L and Tn7R) flanking a baculovirus gene promoter operably linked to a Rep gene. In certain embodiments, the Rep gene is selected from the group consisting of B19 Rep, GPV Rep, HBoV1, and AAV2 Rep. In certain embodiments, the Tn7 transfer vector comprises the left and right ends of Tn7 flanking a polyhedrin promoter operably linked to B19 Rep, as shown in FIGS. 3A and 3B. In certain embodiments, the Tn7 transfer vector comprises the left and right ends of Tn7 flanking an ie1 promoter operably linked to B19 Rep, as shown in FIGS. 3A and 3B. In certain embodiments, the Tn7 transfer vector comprises the left and right ends of Tn7 flanking a polyhedrin promoter operably linked to GPV Rep, as shown in FIGS. 3C and 3D. In certain embodiments, the Tn7 transfer vector comprises the left and right ends of Tn7 flanking a polyhedrin promoter operably linked to AAV2 Rep, as shown in FIGS. 3E and 3F. In certain embodiments, the Tn7 transfer vector comprises the left and right ends of Tn7 flanking a polyhedrin promoter operably linked to HBoV1 Rep (no data provided).

### Cre-LoxP Transfer Vector

In certain embodiments, the transfer vector is a Cre-LoxP transfer vector. In certain embodiments, the Cre-LoxP transfer vector is a nucleic acid vector comprising: a first origin of replication for propagating the nucleic acid vector in a first bacterial strain, wherein the first origin of replication is a conditional origin of replication; a second origin of replication for propagating the nucleic acid vector in a second bacterial strain; a multiple cloning site for the insertion of a foreign sequence (e.g., a heterologous sequence); a selectable marker sequence; a reporter gene; and/or a preferential target site capable of mediating a site-specific recombination event. In certain embodiments, the first origin of replication is conditional upon the presence of a pi protein, such as the R6Kγ origin of replication. In certain embodiments, the first origin of replication is the R6Kγ origin of replication. As such, in certain embodiments, the first bacterial strain comprises a pi protein. In certain embodiments, the second origin of replication is the pUC57 origin of replication. In certain embodiments, the preferential target site capable of mediating a site-specific recombination event comprises a LoxP site or a variant thereof, and the site-specific recombination event is mediated by a Cre recombinase. A Cre-LoxP transfer vector described herein can have any other elements suitable for the proper identification of host cells comprising the vector. For example, any selectable marker sequence and/or reporter gene known to those of skill in the art can be incorporated in a Cre-LoxP transfer vector described herein.

In certain embodiments, the Cre-LoxP transfer vector is a pUC57 vector comprising: a LoxP recombination site, an enhanced GFP (eGFP) marker gene under the AcMNPV ie1 promoter preceded by the transcriptional enhancer hr5 element and followed by the AcMNPV p10 polyadenylation signal, an ampicillin resistance marker, a conditional R6Kγ origin of replication, and a multiple cloning site. The features of the Cre-LoxP transfer vector includes: 1) a multiple cloning site (MCS) for inserting transgenes; 2) a LoxP site for Cre-mediated in-vitro or in-vivo recombination; 3) two origins of replication including: i) a ColE1 Ori for propagating this vector into E. coli strains such as DH5α, NEB Stable, PMC103, and DH10B; and ii) a conditional R6Kγ Ori for propagating this vector into E. coli strains expressing π (pi) protein such as pir+ and pir116; 4) an ampicillin antibiotic-resistant gene for selecting the recombinant bacmid with kanamycin and ampicillin after inserting the Cre-LoxP donor vector in the LoxP site in BIVVBac by Cre mediated recombination; and 5) an enhanced GFP reporter gene for determining the stability of transgenes in the recombinant BEV while replicating in insect cells over serial passages. It will be appreciated by those of skill in the art that whether the multiple cloning site is maintained in a Cre-LoxP transfer vector comprising a foreign sequence (e.g., transgene) depends on how the foreign sequence is inserted therein (e.g., by using the terminal restriction sites or using non-terminal restriction sites, or the nature of the restriction enzyme used to clone the foreign sequence in).

In certain exemplary embodiments, for the purposes of producing a gene therapy vector, the Cre-LoxP transfer vector comprises a foreign sequence (e.g., a heterologous sequence). For example, in certain embodiments, the Cre-LoxP transfer vector comprises a therapeutic protein-coding gene flanked by symmetric or assymetric AAV or non-AAV inverted terminal repeats (ITRs). In certain embodiments, the therapeutic protein-coding gene flanked by symmetric or assymetric AAV or non-AAV ITRs resides in the location of the multiple cloning site of the Cre-LoxP transfer vector.

It will be recognized by those of skill in the art that the Tn7 transfer vector and Cre-LoxP transfer vector are examples of transfer vectors that could be employed for insertion of a foreign sequence to generate a recombinant bacmid of the present invention. In certain embodiments, the foreign sequence that is to be inserted using the transfer vectors described herein are interchangeable between the transfer vectors. For example, for the purposes of producing a gene therapy vector, a Rep gene may be introduced into the recombinant bacmid by the Tn7 transfer vector, and the therapeutic protein-coding gene may be introduced by the Cre-LoxP transfer vector. In another example, for the purposes of producing a gene therapy vector, a Rep gene may be introduced into the recombinant bacmid by the Cre-LoxP transfer vector, and the therapeutic protein-coding gene may be introduced by the Tn7 transfer vector.

In certain embodiments, a Cre-LoxP transfer vector of the present invention comprises a LoxP sequence and the enhanced green fluorescent protein (eGFP) coding gene, and is used for cloning a therapeutic protein-coding gene (e.g., hFVIIIco6XTEN with flanking symmetric or asymmetric ITRs; where the ITRs are of a non-AAV or AAV).

In certain embodiments, a recombinant bacmid is provided, wherein a Rep coding gene is inserted at the mini-attTn7 site in the Polyhedrin locus via transposition, and a therapeutic protein-coding gene with symmetric or asymmetric ITRs is inserted at the LoxP site in the EGT locus via Cre mediated recombination. In certain embodiments, the Rep gene and the ITRs are of a non-AAV or AAV.

Accordingly, in certain embodiments, a recombinant bacmid is provided, comprising: a sequence encoding an AAV Rep (e.g., AAV2 Rep) inserted into a LacZα or functional portion thereof (e.g., inserted into a mini-attTn7 site within the LacZα), wherein the inserted Rep disrupts the reading frame of the LacZα or functional portion thereof; and a multiple cloning site comprising a heterologous sequence, wherein the heterologous sequence comprises from 5′ to 3′: a wild-type or truncated AAV 5′ inverted terminal repeat derived; a sequence encoding a protein; one or more expression control sequences operably linked to the sequence encoding a protein; and a wild-type or truncated AAV 3′ inverted terminal repeat.

In certain embodiments, a recombinant bacmid is provided, comprising: a sequence encoding a non-AAV Rep inserted into a LacZα or functional portion thereof (e.g., inserted into a mini-attTn7 site within the LacZα), wherein the inserted Rep disrupts the reading frame of the LacZα or functional portion thereof; and a multiple cloning site comprising a heterologous sequence, wherein the heterologous sequence comprises from 5′ to 3′: a wild-type or truncated non-AAV 5′ inverted terminal repeat derived from a first parvoviral non-AAV genome; a sequence encoding a protein; one or more expression control sequences operably linked to the sequence encoding a protein; and a wild-type or truncated non-AAV 3′ inverted terminal repeat derived from a second parvoviral non-AAV genome. In certain embodiments, the first and the second parvoviral non-AAV genomes are different.

In certain embodiments, provided herein is a recombinant bacmid comprising: a sequence encoding a B19 Rep inserted into a LacZα or functional portion thereof (e.g., inserted into a mini-attTn7 site within the LacZα), wherein the inserted B19 Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from parvovirus B19; a sequence encoding a protein; one or more expression control sequences operably linked to the sequence encoding a protein; and a wild-type or truncated 3′ inverted terminal repeat derived from parvovirus B19.

In certain embodiments, provided herein is a recombinant bacmid comprising: a sequence encoding a GPV Rep inserted into a LacZα or functional portion thereof (e.g., inserted into a mini-attTn7 site within the LacZα), wherein the inserted GPV Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from GPV; a sequence encoding a protein; one or more expression control sequences operably linked to the sequence encoding a protein; and a wild-type or truncated 3′ inverted terminal repeat derived from GPV.

In certain embodiments, provided herein is a recombinant bacmid comprising: a sequence encoding a AAV2 Rep inserted into a LacZα or functional portion thereof (e.g., inserted into a mini-attTn7 site within the LacZα), wherein the inserted AAV2 Rep disrupts the reading frame of the LacZα or functional portion thereof; and a multiple cloning site comprising a heterologous sequence, wherein the heterologous sequence comprises from 5′ to 3′: a wild-type or truncated 5′ inverted terminal repeat derived from AAV2; a sequence encoding a protein; one or more expression control sequences operably linked to the sequence encoding a protein; and a wild-type or truncated 3′ inverted terminal repeat derived from AAV2.

In certain embodiments, provided herein is a recombinant bacmid comprising: a sequence encoding a HBoV1 Rep inserted into a LacZα or functional portion thereof (e.g., inserted into a mini-attTn7 site within the LacZα), wherein the inserted HBoV1 Rep disrupts the reading frame of the LacZα or functional portion thereof; and a multiple cloning site comprising a heterologous sequence, wherein the heterologous sequence comprises from 5′ to 3′: a wild-type 5′ inverted terminal repeat derived from HBoV1; a sequence encoding a protein; one or more expression control sequences operably linked to the sequence encoding a protein; and a wild-type 3′ inverted terminal repeat derived from HBoV1.

In certain embodiments, a recombinant bacmid is provided, comprising: a sequence encoding a non-AAV Rep inserted into a LacZα or functional portion thereof (e.g., inserted into a mini-attTn7 site within the LacZα), wherein the inserted Rep disrupts the reading frame of the LacZα or functional portion thereof; and a multiple cloning site comprising a heterologous sequence, wherein the heterologous sequence comprises from 5′ to 3′: a wild-type or truncated non-AAV 5′ inverted terminal repeat; a sequence encoding a protein; one or more expression control sequences operably linked to the sequence encoding a protein; and a wild-type or truncated non-AAV 3′ inverted terminal repeat. In certain embodiments the non-AAV Rep and the non-AAV inverted terminal repeats are selected from an ITR of a non-AAV genome from the viral family Parvoviridae. In certain embodiments, the 5′ ITR and the 3′ ITR are of the same parvoviral non-AAV genome. In certain embodiments, the 5′ ITR and the 3′ ITR are of different parvoviral non-AAV genomes. In such an embodiment, the non-AAV Rep is a Rep of at least one of the paroviral non-AAV genomes. In such embodiments, those of skill in the art will be able to determine the most suitable Rep to be used in embodiments where the 5′ ITR and 3′ ITR are of different parvoviral non-AAV genomes.

In certain embodiments, the recombinant bacmid comprises a wild-type or truncated AAV 5′ ITR, and a wild-type or truncated parvoviral non-AAV 3′ ITR. In certain embodiments, the recombinant bacmid comprises a wild-type or truncated parvoviral non-AAV 5′ ITR, and a wild-type or truncated AAV 3′ ITR. In such embodiments, those of skill in the art will be able to determine the most suitable Rep to be used in embodiments where the 5′ ITR and 3′ ITR are of different parvoviral genomes.

### III. Foreign Sequences

It will be appreciated to those of skill in the art, that the baculovirus expression vector system described herein can be used for the production of any desired product. For example, the baculovirus expression vector system described herein can be used for the production of a recombinant protein, or a viral or non-viral vector for gene therapeutics. As such, the following description of the production of nucleic acid molecules should not be limiting in any way.

### Nucleic Acid Molecules

In certain embodiments, the baculovirus expression vector system described herein can be used for the production of nucleic acid molecules. For example, the production of nucleic acid molecules for gene therapy. As such, certain embodiments described herein relate to the production of a plasmid-like, capsid free, nucleic acid molecule that encodes a target sequence. A capsid, the protein shell of a virus, encloses the genetic material of the virus. Capsids are known to aid the functions of the virion by protecting the viral genome, delivering the genome to a host, and interacting with the host. Nonetheless, the viral capsids may be a factor in limiting the packaging capacity of the vectors and/or inducing immune responses, especially when used in gene therapy.

AAV vectors have emerged as one of the more common types of gene therapy vectors. However, the presence of the capsid limits the utility of an AAV vector in gene therapy. In particular, the capsid itself can limit the size of the transgene that is included in the vector to as low as less than 4.5 kb. Various therapeutic proteins that may be useful in a gene therapy can easily exceed this size even before expression control sequences are added.

Furthermore, proteins that make up the capsid can serve as antigens that can be targeted by a subject's immune system. AAV is very common in the general population, with most people having been exposed to an AAV throughout their lives. As a result, most potential gene therapy recipients have likely already developed an immune response to an AAV, and thus are more likely to reject the therapy.

In certain embodiments, the present invention is directed to the production of a nucleic acid molecule, comprising a first ITR, a second ITR, and a genetic cassette, e.g., encoding a therapeutic protein and/or a miRNA. In some embodiments, the first ITR and second ITR flank a genetic cassette comprising a heterologous polynucleotide sequence. In some embodiments, the nucleic acid molecule does not comprise a gene encoding a capsid protein, a replication protein, and/or an assembly protein. In some embodiments, the genetic cassette encodes a therapeutic protein. In some embodiments, the therapeutic protein comprises a clotting factor. In some embodiments, the genetic cassette encodes a miRNA. In certain embodiments, the genetic cassette is positioned between the first ITR and the second ITR. In some embodiments, the nucleic acid molecule further comprises one or more noncoding region. In certain embodiments, the one or more non-coding region comprises a promoter sequence, an intron, a post-transcriptional regulatory element, a 3′UTR poly(A) sequence, or any combination thereof.

In one embodiment, the genetic cassette is a single stranded nucleic acid. In another embodiment, the genetic cassette is a double stranded nucleic acid.

Some aspects of the present disclosure are directed to a nucleic acid molecule comprising a genetic cassette, e.g., encoding a therapeutic protein and/or a miRNA. In some embodiments, the genetic cassette encodes a therapeutic protein. In some embodiments, the therapeutic protein comprises a clotting factor. In some embodiments, the genetic cassette encodes a miRNA. In some embodiments, the nucleic acid molecule further comprises at least one noncoding region. In certain embodiments, the at least one non-coding region comprises a promoter sequence, an intron, a post-transcriptional regulatory element, a 3′UTR poly(A) sequence, or any combination thereof.

In some embodiments, the nucleic acid molecules disclosed herein comprise an intron or intronic sequence. In some embodiments, the intronic sequence is a naturally occurring intronic sequence. In some embodiments, the intronic sequence is a synthetic sequence. In some embodiments, the intronic sequence is derived from a naturally occurring intronic sequence. In some embodiments, the intronic sequence is a hybrid synthetic intron or chimeric intron. In some embodiments, the intronic sequence is a chimeric intron that consists of chicken beta-actin/rabbit beta-globin intron and has been modified to eliminate five existing ATG sequences to reduce false translation starts. In certain embodiments, the intronic sequence comprises the SV40 small T intron. In some embodiments, the intronic sequence is positioned 5′ to the nucleic acid sequence encoding the FVIII polypeptide. In some embodiments, the chimeric intron is positioned 5′ to a promoter sequence, such as the mTTR promoter. In some embodiments, the chimeric intron comprises the nucleic acid sequence of SEQ ID NO: 23.

In one embodiment, the genetic cassette is a single stranded nucleic acid. In another embodiment, the genetic cassette is a double stranded nucleic acid. In another embodiment, the genetic cassette is a closed-end double stranded nucleic acid (ceDNA).

In some embodiments, the genetic cassette comprises a nucleotide sequence encoding a FVIII polypeptide, wherein the nucleotide sequence is codon optimized. In some embodiments, the genetic cassette comprises a nucleotide sequence encoding a codon optimized FVIII driven by a mTTR promoter and synthetic intron. In some embodiments, the genetic cassette comprises a nucleotide sequence which is disclosed in International Application No. PCT/US2017/015879, which is incorporated by reference in its entirety. In some embodiments, the genetic cassette is a “hFVIIIco6XTEN” genetic cassette as described in PCT/US2017/015879. In some embodiments, the genetic cassette comprises codon optimized cDNA encoding B-domain deleted (BDD) codon-optimized human Factor VIII (BDDcoFVIII) fused with XTEN 144 peptide.

In some embodiments, the genetic cassette comprises a nucleotide sequence encoding a codon optimized FVIII driven by a mTTR promoter. In some embodiments, the mTTR promoter comprises the nucleic acid sequence of SEQ ID NO: 22. In some embodiments, the genetic cassette further comprises an A1MB2 enhancer element. In some embodiments, the A1MB2 enhancer element comprises the nucleic acid sequence of SEQ ID NO: 21. In some embodiments, the genetic cassette further comprises a chimeric or synthetic intron. In some embodiments, the chimeric intron consists of chicken beta-actin/rabbit beta-globin intron and has been modified to eliminate five existing ATG sequences to reduce false translation starts. In some embodiments, the intronic sequence is positioned 5′ to the nucleic acid sequence encoding the FVIII polypeptide. In some embodiments, the chimeric intron is positioned 5′ to a promoter sequence, such as the mTTR promoter. In some embodiments, the chimeric intron comprises the nucleic acid sequence of SEQ ID NO: 23. In some embodiments, the genetic cassette further comprises a a Woodchuck Posttranscriptional Regulatory Element (WPRE). In some embodiments, the WPRE comprises the nucleic acid sequence of SEQ ID NO: 24. In some embodiments, the genetic cassette further comprises a Bovine Growth Hormone Polyadenylation (bGHpA) signal. In some embodiments, the bGHpA signal comprises the nucleic acid sequence of SEQ ID NO: 25. In some embodiments, the genetic cassette comprises a nucleotide sequence having at least 70%, 75%, 80%, 85%, 90%, 95%, or 100% sequence identity to SEQ ID NO: 19. In some embodiments, the genetic cassette comprises the nucleotide sequence of SEQ ID NO: 19.

In some embodiments, the genetic cassette comprises a nucleotide sequence encoding a codon optimized FVIII and XTEN peptide driven by a liver-specific modified mouse transthyretin (mTTR) promoter (mTTR482) which includes an A1MB2 enhancer element, hybrid synthetic intron (chimeric Intron), the Woodchuck Posttranscriptional Regulatory Element (WPRE), and the Bovine Growth Hormone Polyadenylation (bGHpA) signal. In some embodiments, the genetic cassette comprises the nucleotide sequence of SEQ ID NO: 19. In some embodiments, the genetic cassette comprises a nucleotide sequence having at least 70%, 75%, 80%, 85%, 90%, 95%, or 100% sequence identity to SEQ ID NO: 19.

### Inverted Terminal Repeats

In certain embodiments, the 5′ ITR and the 3′ ITR are adeno-associated virus (AAV) ITRs or non-AAV ITRs. In certain embodiments, non-AAV ITRs are ITRs obtained from a member of the viral family Parvoviridae. Suitable ITR sequences include AAV ITRs of AAV serotypes known to those of skill in the art. Suitable AAV and non-AAV ITR sequences are described in PCT Publication Nos. WO2019032898A1, WO2020033863A1, and WO2017152149A1, the disclosures of which are herein incorporated by reference in their entireties. For example, non-AAV ITR sequences may be derived from Goose parvovirus (GPV) or parvovirus B19 (also referred to herein as “B19”). In some embodiments, the ITR is not derived from an AAV genome. In some embodiments, the ITR is an ITR of a non-AAV. In some embodiments, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain embodiments, the ITR is derived from erythrovirus parvovirus B19 (human virus; also referred to herein as “B19”)). In another embodiment, the ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY. In some embodiments, the ITR is derived from a porcine parvovirus, e.g., porcine parvovirus U44978. In some embodiments, the ITR is derived from a mice minute virus, e.g., mice minute virus U34256. In some embodiments, the ITR is derived from a canine parvovirus, e.g., canine parvovirus M19296. In some embodiments, the ITR is derived from a mink enteritis virus, e.g., mink enteritis virus D00765. In some embodiments, the ITR is derived from a Dependoparvovirus. In one embodiment, the Dependoparvovirus is a Dependovirus Goose parvovirus (GPV) strain. In a specific embodiment, the GPV strain is attenuated, e.g., GPV strain 82-0321V. In another specific embodiment, the GPV strain is pathogenic, e.g., GPV strain B. Examples of suitable Parvoviral ITR sequences are set forth in Table 1.

TABLE 1
Parvoviral ITR Sequences

| SEQ | | | |
|---|---|---|---|
| ID | | | |
| NO: | Parvovirus | Descriptor | Sequence |
| 1 | B19 | B19Δ135 | CTCTGGGCCAGCTTGCTTGGGGTTGCCTTGACACTAAGA |
| | | | CAAGCGGCGCGCCGCTTGATCTTAGTGGCACGTCAACCC |
| | | | CAAGCGCTGGCCCAGAGCCAACCCTAATTCCGGAAGTCC |
| | | | CGCCCACCGGAAGTGACGTCACAGGAAATGACGTCACA |
| | | | GGAAATGACGTAATTGTCCGCCATCTTGTACCGGAAGTC |
| | | | CCGCCTACCGGCGGCGACCGGCGGCATCTGATTTGGTG |
| | | | TCTTCTTTTAAATTTT |
| 2 | | B19.WT | CCAAATCAGATGCCGCCGGTCGCCGCCGGTAGGCGGGA |
| | | | CTTCCGGTACAAGATGGCGGACAATTACGTCATTTCCTGT |
| | | | GACGTCATTTCCTGTGACGTCACTTCCGGTGGGCGGGAC |
| | | | TTCCGGAATTAGGGTTGGCTCTGGGCCAGCTTGCTTGGG |
| | | | GTTGCCTTGACACTAAGACAAGCGGCGCGCCGCTTGATC |
| | | | TTAGTGGCACGTCAACCCCAAGCGCTGGCCCAGAGCCAA |
| | | | CCCTAATTCCGGAAGTCCCGCCCACCGGAAGTGACGTCA |
| | | | CAGGAAATGACGTCACAGGAAATGACGTAATTGTCCGCC |
| | | | ATCTTGTACCGGAAGTCCCGCCTACCGGCGGCGACCGG |
| | | | CGGCATCTGATTTGGTGTCTTCTTTTAAATTTT |
| 3 | GPV | GPVΔ162 | CGGTGACGTGTTTCCGGCTGTTAGGTTGACCACGCGCAT |
| | | | GCCGCGCGGTCAGCCCAATAGTTAAGCCGGAAACACGT |
| | | | CACCGGAAGTCACATGACCGGAAGTCACGTGACCGGAAA |
| | | | CACGTGACAGGAAGCACGTGACCGGAACTACGTCACCG |
| | | | GATGTGCGTCACCGGAAGCATGTGACCGGAACTTGCGTC |
| | | | ACTTCCCCCTCCCCTGATTGGCTGGTTCGAACGAACGAA |
| | | | CCCTCCAATGAGACTCAAGGACAAGAGGATATTTTGCGC |
| | | | GCCAGGAAGTG |
| 4 | | GPV.WT | CTCATTGGAGGGTTCGTTCGTTCGAACCAGCCAATCAGG |
| | | | GGAGGGGGAAGTGACGCAAGTTCCGGTCACATGCTTCC |
| | | | GGTGACGCACATCCGGTGACGTAGTTCCGGTCACGTGCT |
| | | | TCCTGTCACGTGTTTCCGGTCACGTGACTTCCGGTCATG |
| | | | TGACTTCCGGTGACGTGTTTCCGGCTGTTAGGTTGACCA |
| | | | CGCGCATGCCGCGCGGTCAGCCCAATAGTTAAGCCGGA |
| | | | AACACGTCACCGGAAGTCACATGACCGGAAGTCACGTGA |
| | | | CCGGAAACACGTGACAGGAAGCACGTGACCGGAACTAC |
| | | | GTCACCGGATGTGCGTCACCGGAAGCATGTGACCGGAA |
| | | | CTTGCGTCACTTCCCCCTCCCCTGATTGGCTGGTTCGAA |
| | | | CGAACGAACCCTCCAATGAGACTCAAGGACAAGAGGATA |
| | | | TTTTGCGCGCCAGGAAGTG |
| 5 | AAV2 | AAV2.WT (5′) | AGGAACCCCTAGTGATGGAGTTGGCCACTCCCTCTCTGC |
| | | | GCGCTCGCTCGCTCACTGAGGCCGCCCGGGCAAAGCCC |
| | | | GGGCGTCGGGCGACCTTTGGTCGCCCGGCCTCAGTGAG |
| | | | CGAGCGAGCGCGCAGAGAGGGAGTGGCCAA |
| 6 | | AAV2.WT (3′) | AGGAACCCCTAGTGATGGAGTTGGCCACTCCCTCTCTGC |
| | | | GCGCTCGCTCGCTCACTGAGGCCGGGCGACCAAAGGTC |
| | | | GCCCGACGCCCGGGCTTTGCCCGGGCGGCCTCAGTGAG |
| | | | CGAGCGAGCGCGCAGAGAGGGAGTGGCCAA |
| 7 | | AAV2Δ15 | AGGAACCCCTAGTGATGGAGTTGGCCACTCCCTCTCTGC |
| | | | GCGCTCGCTCGCTCACTGAGGCCGGGCGACCAAAGGTC |
| | | | GCCCGACGCCCGGGCTTTGCCCGGGCGGCCTCAGTGAG |
| | | | CGAGCGAGCGCGCAG |
| 8 | | AAV2Δ15Δ11 | AGGAACCCCTAGTGATGGAGTTGGCCACTCCCTCTCTGC |
| | | | GCGCTCGCTCGCTCACTGAGGCCGGGCGACCAAAGGTC |
| | | | GCCCGACGCCCGGGCGGCCTCAGTGAGCGAGCGAGCG |
| | | | CGCAG |
| 26 | HBoV1 | HBoV1 5′ ITR | GTGGTTGTACAGACGCCATCTTGGAATCCAATATGTCTGC |
| | | | CGGCTCAGTCATGCCTGCGCTGCGCGCAGCGCGCTGCG |
| | | | CGCGCGCATGATCTAATCGCCGGCAGACATATTGGATTC |
| | | | CAAGATGGCGTCTGTACAACCAC |
| 27 | | HBoV1 3′ ITR | TTGCTTATGCAATCGCGAAACTCTATATCTTTTAATGTGTT |
| | | | GTTGTTGTACATGCGCCATCTTAGTTTTATATCAGCTGGC |
| | | | GCCTTAGTTATATAACATGCATGTTATATAACTAAGGCGC |
| | | | CAGCTGATATAAAACTAAGATGGCGCATGTACAACAACAA |
| | | | CACATTAAAAGATATAGAGTTTCGCGATTGCATAAGCAA |

### Therapeutic Proteins

In some aspects, provided herein is the production of a nucleic acid molecule comprising a first ITR, a second ITR, and a genetic cassette encoding a target sequence, wherein the target sequence encodes a therapeutic protein. In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

Certain embodiments of the present disclosure are directed to a nucleic acid molecule comprising a first ITR, a second ITR, and a genetic cassette encoding a therapeutic protein. Any therapeutic protein can be produced by a baculovirus expression vector system of the present disclosure, including, without limitation, production of clotting factor. In some embodiments, the clotting factor is selected from the group consisting of FI, FII, FIII, FIV, FV, FVI, FVII, FVIII, FIX, FX, FXI, FXII, FXIII, VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In one embodiment, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

### Growth Factors

In some aspects, provided herein is the production of a nucleic acid molecule comprising a first ITR, a second ITR, and a genetic cassette encoding a target sequence, wherein the target sequence encodes a therapeutic protein, and wherein the therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7. In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin B1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is foetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

Micro RNAs (miRNAs)

MicroRNAs (miRNAs) are small non-coding RNA molecules (about 18-22 nucleotides) that negatively regulate gene expression by inhibiting translation or inducing messenger RNA (mRNA) degradation. Since their discovery, miRNAs have been implicated in various cellular processes including apoptosis, differentiation and cell proliferation and they have shown to play a key role in carcinogenesis. The ability of miRNAs to regulate gene expression makes expression of miRNAs in vivo a valuable tool in gene therapy.

In some aspects, provided herein is the production of a nucleic acid molecule comprising a first ITR, a second ITR, and a genetic cassette encoding a target sequence, wherein the target sequence encodes a miRNA, and wherein the first ITR and/or the second ITR are an ITR of a non-adeno-associated virus (e.g., the first ITR and/or the second ITR are from a non-AAV). The miRNA can be any miRNA known in the art. In some embodiments, the miRNA down regulates the expression of a target gene. In certain embodiments, the target gene is selected from SOD1, HTT, RHO, or any combination thereof.

In some embodiments, the genetic cassette encodes one miRNA. In some embodiments, the genetic cassette encodes more than one miRNA. In some embodiments, the genetic cassette encodes two or more different miRNAs. In some embodiments, the genetic cassette encodes two or more copies of the same miRNA. In some embodiments, the genetic cassette encodes two or more variants of the same therapeutic protein. In certain embodiments, the genetic cassette encodes one or more miRNA and one or more therapeutic protein.

In some embodiments, the miRNA is a naturally occurring miRNA. In some embodiments, the miRNA is an engineered miRNA. In some embodiments, the miRNA is an artificial miRNA. In certain embodiments, the miRNA comprises the miHTT engineered miRNA disclosed by Evers et al., Molecular Therapy 26(9):1-15 (epub ahead of print June 2018). In certain embodiments, the miRNA comprises the miR SOD1 artificial miRNA disclosed by Dirren et al., Annals of Clinical and Translational Neurology 2(2):167-84 (February 2015). In certain embodiments, the miRNA comprises miR-708, which targets RHO (see Behrman et al., JCB 192(6):919-27 (2011).

In some embodiments, the miRNA upregulates expression of a gene by down regulating the expression of an inhibitor of the gene. In some embodiments, the inhibitor is a natural, e.g., wild-type, inhibitor. In some embodiments, the inhibitor results from a mutated, heterologous, and/or misexpressed gene.

### Expression Control Elements

In some embodiments, the nucleic acid molecule or vector produced by a baculovirus expression vector system described herein further comprises at least one expression control sequence. An expression control sequence as used herein is any regulatory nucleotide sequence, such as a promoter sequence or promoter-enhancer combination, which facilitates the efficient transcription and translation of the coding nucleic acid to which it is operably linked. For example, the isolated nucleic acid molecule produced by a method of the disclosure can be operably linked to at least one transcription control sequence.

The gene expression control sequence can, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus.

Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. Examples of liver specific promoters include, but are not limited to, a mouse transthyretin promoter (mTTR), a native human factor VIII promoter, a native human factor IX promoter, human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The F8 promoter is described in Figueiredo and Brownlee, 1995, J. Biol. Chem. 270:11828-11838. In certain embodiments, the promoter comprises any of the mTTR promoters (e.g., mTTR202 promoter, mTTR202opt promoter, mTTR482 promoter) as disclosed in U.S. patent publication no. US2019/0048362, which is incorporated by reference herein in its entirety.

In some embodiments, the nucleic acid molecule comprises a tissue specific promoter. In certain embodiments, the tissue specific promoter drives expression of the therapeutic protein, e.g., the clotting factor, in the liver, e.g., in hepatocytes and/or endothelial cells. In particular embodiments, the promoter is selected from the group consisting of a mouse transthyretin promoter (mTTR), a native human factor VIII promoter, a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, a phosphoglycerate kinase (PGK) promoter and any combination thereof. In some embodiments, the promoter is selected from a liver specific promoter (e.g., al-antitrypsin (AAT)), a muscle specific promoter (e.g., muscle creatine kinase (MCK), myosin heavy chain alpha (aMHC), myoglobin (MB), and desmin (DES)), a synthetic promoter (e.g., SPc5-12, 2R5Sc5-12, dMCK, and tMCK) and any combination thereof.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In a particular example, a promoter useful for the disclosure is an ET promoter, which is also known as GenBank No. AY661265. See also Vigna et al., Molecular Therapy 11(5):763 (2005). Examples of other suitable vectors and expression control sequences are described in WO 02/092134, EP1395293, or U.S. Pat. Nos. 6,808,905, 7,745,179, or 7,179,903, which are incorporated by reference herein in their entireties.

In general, the expression control sequences shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

In certain embodiments, a nucleic acid molecule produced by a baculovirus expression vector system described herein comprises one or more miRNA target sequences which, for example, are operably linked to a transgene.

In some embodiments, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In some embodiments, the target sequence is an miR-142 target. In some embodiments, the complementary sequence of hematopoietic-specific microRNAs, such as miR-142 (142T), is incorporated into a nucleic acid molecule comprising a transgene, making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can impose a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100 complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### Heterologous Moieties

In some embodiments, the transgene encodes a heterologous amino acid sequence. The heterologus amino acid sequence can be linked to the transgene product. In some embodiments, the heterologous amino acid sequence can be linked to the N-terminus or the C-terminus of the transgene product. For example, with respect to a transgene encoding FVIII, the heterologous amino acid sequence can be linked to the N-terminus or the C-terminus of the FVIII amino acid sequence or inserted between two amino acids in the FVIII amino acid sequence. In some embodiments, the heterologous amino acid sequence can be inserted within the FVIII polypeptide at any site disclosed in International Publication No. WO 2013/123457 A1 and WO 2015/106052 A1 or U.S. Publication No. 2015/0158929 A1, which are herein incorporated by reference in their entirety.

In some embodiments, the heterologous amino acid sequence is inserted within the B domain of FVIII or a fragment thereof.

In some embodiments, a heterologous moiety comprises one or more XTEN sequences, fragments, variants, or derivatives thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

In some embodiments, the XTEN sequence useful for the disclosure is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence of the disclosure can comprise one or more sequence motifs of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine (S), threonine (T), glutamate (E) and proline (P). Examples of XTEN sequences that can be used as heterologous moieties in chimeric proteins of the disclosure are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010/091122 A1, WO 2010/144502 A2, WO 2010/144508 A1, WO 2011/028228 A1, WO 2011/028229 A1, or WO 2011/028344 A2, each of which is incorporated by reference herein in its entirety.

### IV. Host Cells

Suitable host cells are known to those of skill in the art. A “host cell” refers to any cell that harbors, or is capable of harboring, any substance of interest.

In some embodiments, host cells suitable for use in the present invention are of insect origin. In some embodiments, a suitable insect host cell includes, for example, a cell line isolated from Spodoptera frugiperda (Sf) or a cell line isolated from Trichoplusia ni (Tni). Those of skill in the art will readily be able to determine the suitability of any Sf or Tni cell line. Exemplary insect host cells include, without limitation, Sf9 cells, Sf21 cells, a High Five™ cells. Exemplary insect host cells also include, without limitation, any Sf or Tni cell line that is free from adventitious virus contamination, e.g., Sf-rhadovirus-negative (Sf-RVN) and Tn-nodavirus-negative (Tn-NVN) cells. Other suitable host insect cells are known to those of skill in the art. In one particular embodiment, the insect host cells are Sf9 cells.

In some embodiments, host cells suitable for use in the present invention are of bacterial origin. In some embodiments, a suitable bacterial host cell can be any bacterial host cell known to those of skill in the art. In certain embodiments, a suitable bacterial host cell is incapable of resolving cruciform DNA structures. In certain embodiments, a suitable bacterial host strain comprises a disruption in the SbcCD complex. In some embodiments, the disruption in the SbcCD complex comprises a genetic disruption in the SbcC gene and/or SbcD gene. In certain embodiments, the disruption in the SbcCD complex comprises a genetic disruption in the SbcC gene. Various bacterial host strains that comprise a genetic disruption in the SbcC gene are known in the art. For example, without limitation, the bacterial host strain PMC103 comprises the genotype sbcC, recD, mcrA, ΔmcrBCF; the bacterial host strain PMC107 comprises the genotype recBC, recJ, sbcBC, mcrA, ΔmcrBCF; and the bacterial host strain SURE comprises the genotype recB, recJ, sbcC, mcrA, ΔmcrBCF, umuC, uvrC.

In some embodiments, host cells suitable for use in the present invention are of mammalian origin, e.g., human origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/0 (mouse myeloma), P3.times.63-Ag3.653 (mouse myeloma), BFA-1c1BPT (bovine endothelial cells), RAJI (human lymphocyte), PER.C6®, NS0, CAP, BHK21, and HEK 293 (human kidney).

Introduction of the vectors of the disclosure into a host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988).

Host cells comprising vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). The growth medium will generally select for cells containing the vector by, for example, drug selection or deficiency in an essential nutrient which is complemented by the selectable marker on the vector.

### V. Methods of Use

A baculovirus expression vector system provided herein finds use in the production of a product encoded by a foreign sequence inserted in a recombinant bacmid described herein. Scalable production of the product can be achieved by several approaches known in the art.

One approach comprises the infection of suitable insect host cells that supports the growth of baculovirus. In certain embodiments, a recombinant bacmid comprising foreign sequences are described herein is first propagated in a suitable bacterial host cell (e.g., E. coli). The recombinant bacmid is then isolated from the bacterial host cell and transfected into a suitable insect host cell using a suitable transfection reagent (e.g., CELLFECTIN). The insect host cell generates recombinant baculovirus particles which can then be infected into a host insect cell for viral amplification of the foreign sequences.

In certain embodiments, provided herein is a method of producing a product encoded by a foreign sequence, comprising transfecting a recombinant bacmid described herein into a suitable insect cell under appropriate conditions to generate a recombinant baculovirus; and infecting a second suitable insect cell with the recombinant baculovirus under appropriate conditions to produce the product encoded by the foreign sequence. In certain embodiments, for the purposes of producing a gene therapeutic, the recombinant bacmid comprises a Rep coding sequence and a sequence encoding a protein flanked on both sides by ITRs.

In certain embodiments, provided herein is a method of producing a nucleic acid molecule, comprising transfecting a recombinant bacmid described herein into a suitable insect cell under appropriate conditions to generate a recombinant baculovirus; and infecting a second suitable insect cell with the recombinant baculovirus under appropriate conditions to produce the nucleic acid molecule. In certain embodiments, provided herein is a method of producing a ceDNA, comprising transfecting a recombinant bacmid described herein into a suitable insect cell under appropriate conditions to generate a recombinant baculovirus; and infecting a second suitable insect cell with the recombinant baculovirus under appropriate conditions to produce the ceDNA.

In another approach, a stable cell line can be generated by stably integrating a protein encoding sequence under the control of a baculovirus gene promoter (e.g., a baculovirus constitutive gene promoter). In certain embodiments, the stable cell line is a stable insect cell line. Stable integration of sequences can be performed by any method known to those of skill in the art. Methods for stable integration of nucleic acids into a variety of host cell lines are known in the art (see Examples below for more detailed description of an exemplary producer cell line created by stable integration of nucleic acids). For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A. C. et al. (2000) Proc. Natl. Acad. Sci. 97:5995-6000).

In the stable cell line approach, in one embodiment, a BEV encoding a complement protein required for proper expression of the protein encoding sequence is introduced into the stable cell line. In certain embodiments, for the purposes of producing a gene therapeutic, the stable cell line comprises a therapeutic protein-coding gene with flanking symmetric or assymetric AAV or non-AAV ITRs stably integrated therein. A BEV comprising encoding a suitable Rep is then introduced into the stable cell line under conditions necessary for the production of the gene therapeutic.

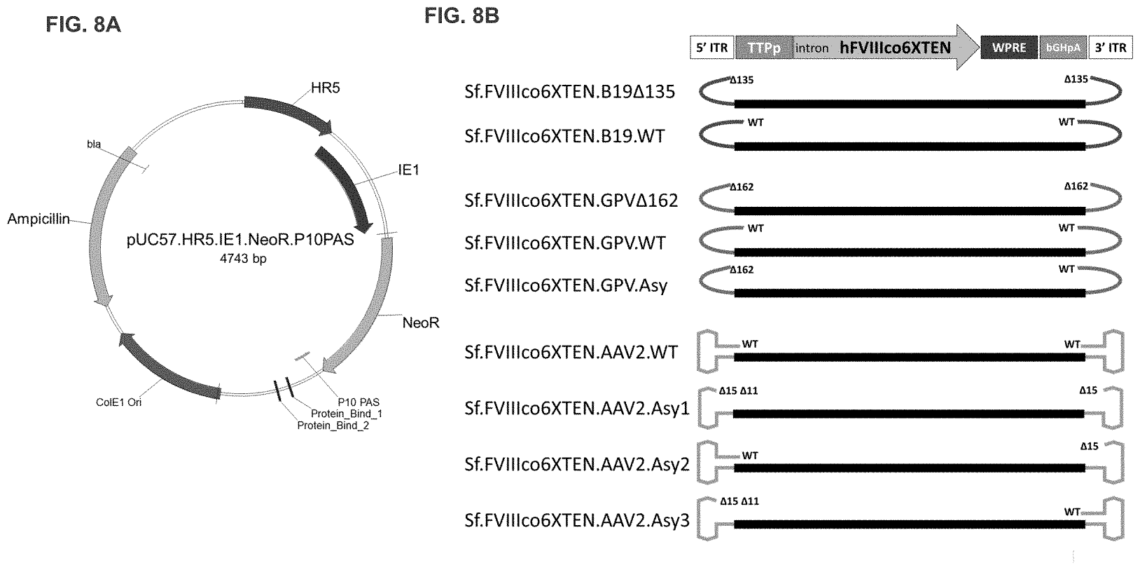

Methods of generating specific stable cell lines are described herein (see, Example 8). Examples of specific stable cell lines for producing ceDNA include cell lines having the plasmids shown in FIG. 8B stably integrated therein. These stable cell lines are listed in Table 5. In certain methods, to produce a ceDNA, a BEV encoding a suitable Rep is introduced into a stable cell line under conditions necessary for the production of the ceDNA.

In yet another approach, production of product encoded by a foreign sequence can be achieved using a stable cell line in a baculovirus-free manner. In certain embodiments, for the purposes of producing a gene therapeutic, the stable cell line comprises a therapeutic protein-coding gene with flanking symmetric or assymetric AAV or non-AAV ITRs stably integrated therein. In certain embodiments, baculovirus-free production in the stable cell lines comprises transient expression of a Rep protein in the stable cell line under the control of a baculovirus gene promoter. Suitable baculovirus gene promoters are known to those of skill in the art. In certain embodiments, the baculovirus gene promoter is an immediate-early (ie) gene promoter of Orgyia pseudotsugata multiple nucleopolyhedrovirus (OpMNPV). In certain embodiments, the baculovirus gene promoter is the OpIE2 promoter of OpMNPV. Various methods of mediating transient gene expression are known to those of skill in the art. In certain embodiments, transient gene expression can be achieved by polyethylenimine (PEI)-mediated trasfection.

Downstream purification of the product can involve any methods known to those of skill in the art. For example, for viral or non-viral vectors for gene therapy purposes may be purified by plasmid DNA isolation kits containing silica-based column that separate the low molecular weight DNA from the RNAs, high molecular weight DNAs, proteins, and other impurities by ion-exchange chromatography.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1: BIVVBac Precursor Bacmid

Among expression vectors, baculovirus stands out because of their outsized genetic cargo capacity—up to several 10s of kb, with reports of 100 kb. This transgene capacity has been taken advantage of for the production of recombinant AAV vectors (up to 38 kb expression cassettes). It was reasoned that the large transgene capacity of the baculovirus vector can also be leveraged for DNA therapeutic drug substance production for non-viral gene therapy. Thus, a new versatile baculovirus shuttle vector (Bacmid) was generated specifically designed to accommodate multiple transgenes than could be accomplished with the existing bacmid tools. This versatile bacmid (called “BIVVBac”) could also be used for rAAV vector production for in vivo gene therapy, as well as for the production of any desired protein, e.g., a recombinant protein.

BIVVBac bacmid was derived from the bMON14272 (Invitrogen) encoding AcMNPV C6 genome (FIG. 1A) and engineered to encode two insertion sites: 1) a mini-attTn7 in the Polyhedrin locus, and 2) a LoxP in the EGT locus. The mini-attTn7 insertion sequence is fused in-frame with a Lac-promoter-driven E. coli LacZα fragment for the X-gal-mediated blue/white screening of recombinant bacmid following Tn7-mediated transposition and the LoxP site for Cre-mediated in-vitro or in-vivo recombination. The LoxP recombination site was inserted in the bMON14272 containing, a mini-attTn7 insertion sequence in the Polyehdrin locus, in two steps. First, a plasmid comprising of a Rudolph Red Fluorescent Protein (RFP) gene under the AcMNPV 39K promoter followed by the ets polyadenylation signal, a LoxP recombination site, and sequences flanking the AcMNPV EGT locus was synthesized through GenScript® (Piscataway, N.J.) (FIG. 1B) (SEQ ID NO:9). Then, the synthetic plasmid DNA was co-transfected with Avrll linearized bMON14272 in Sf9 cells (ATCC® CRL-1711) using Cellfectin (Invitrogen) transfection reagent, according to the manufacturer's instructions. The RFP expression cassette and LoxP sequence were recombined at the EGT locus by the homologous recombination in Sf9 cells. The progeny virus produced in the cell-free supernatant was plaque purified and RFP+ plaques were amplified in Sf9 cells to generate P1 (Passage 1) virus. The baculoviral DNA was then isolated from the P1 virus and used as a template to PCR amplify the regions spanning across the expected recombination junctions using primers internal and external to the transfer plasmid and sequencing the resulting amplimers. Recombinant virus was further amplified, and used for viral DNA isolation, which was then transformed into E. coli DH10B bacteria (O'Reilly et al. 1992), followed by selection with kanamycin, X-Gal, and IPTG. Bacmid DNA isolated from the kanamycin resistant blue E. coli clone was analyzed by restriction enzyme mapping and designated “BIVVBac” (FIG. 1C). A helper plasmid, pMON7124 (Invitrogen) encoding the Tn7 transposase genes tnsA-E and tetracycline resistance was transformed into DH10B E. coli harboring BIVVBac, followed by selection with kanamycin and tetracycline. One of the double resistant clones was used to make electro-competent cells, as described previously (Sharma and Schimke, 1996) and the resulting new E. coli strain was designated BIVVBac^{DH10B} (FIG. 1D). This strain was then used for inserting AAV2, B19, or GPV Replication (Rep) protein-coding gene at the Polyhedrin locus by the Tn7 transposition, as described below.

### Example 2: Cre-LoxP Donor Vector

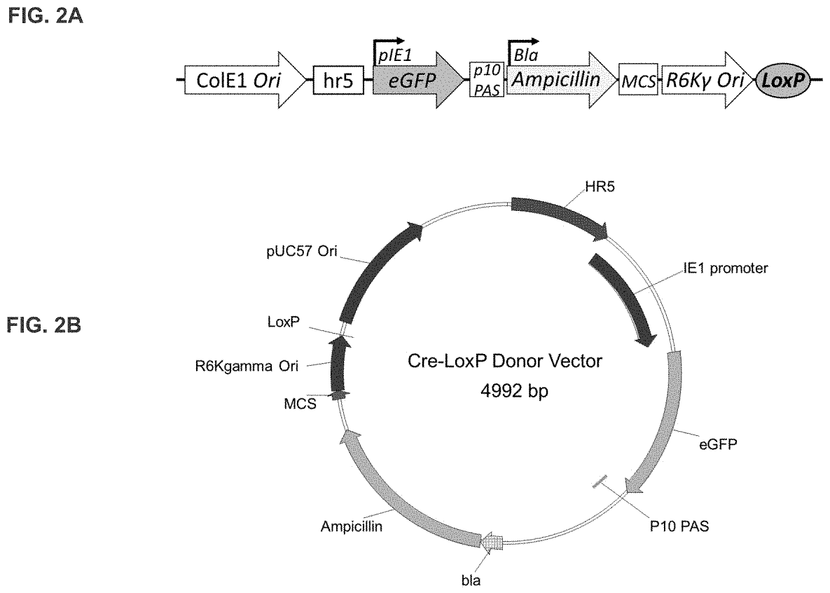

A DNA construct comprising of a LoxP recombination site, an enhanced GFP (eGFP) marker gene under the AcMNPV ie1 promoter preceded with the transcriptional enhancer hr5 element and followed by the AcMNPV p10 polyadenylation signal, an ampicillin resistance marker, a conditional R6Kγ origin of replication, and multiple cloning site was synthesized through GenScript® (Piscataway, N.J.) (SEQ ID NO:10) (FIG. 2A). This synthetic DNA was cloned into the pUC57 vector and the resulting construct was designated “Cre-LoxP donor vector” (FIG. 2B). Key features of the Cre-LoxP donor vector are: (1) A Multiple cloning site (MCS) for inserting transgenes; (2) A LoxP site for Cre-mediated in-vitro or in-vivo recombination; (3) Two origins of replications: (3a) A ColE1 Ori for propagating this vector into E. coli strains such as DH5α, NEB Stable, PMC103, and DH10B, and (3b) A conditional R6Kγ Ori for propagating this vector into E. coli strains expressing π protein such as pir+ and pir116; (4) An ampicillin antibiotic-resistant gene for selecting the recombinant bacmid with kanamycin and ampicillin after inserting the Cre-LoxP donor vector in the LoxP site in ‘BIVVBac’ by Cre-LoxP recombination; and (5) An enhanced GFP reporter gene for determining the stability of transgenes in the recombinant BEV while replicating in Sf9 cells over serial passages.

### Example 3: Replication (Rep) Protein Expression Constructs

### B19.Rep:

The coding sequence of B19 Rep was obtained from B19 strain HV (GenBank accession no. AF162273) and the wild-type sequence was synthesized through GenScript® (Piscataway, N.J.) (SEQ ID NO:11) (FIG. 3A). The synthetic DNA was cloned into pFastBac1 (Invitrogen) vector under the AcMNPV polyhedrin promoter and the gentamycin resistant clone was used to generate pFastBac.Polh.B19.Rep transfer vector. This vector was transformed into BIVVBac^{DH10B} E. coli to produce recombinant BEV, AcBIVVBac.Polh.B19.Rep^{Tn7}, as described below. Titrated BEV was then used for infection in polyclonal Sf cell lines encoding hFVIIIco6XTEN with symmetric B19 ITRs and the ceDNA vector was purified from the infected cell pellets, as described below. Preliminary results revealed that the polyhedrin promoter-driven B19.Rep was unable to ‘rescue’ ceDNA. It was hypothesized that the high level of non-spliced B19.Rep expression at a very-late phase of the baculovirus infection cycle is inefficient and/or toxic to the cells. To this hypothesis, the polyhedrin promoter was replaced with an AcMNPV-constitutive immediate-early (ie1) promoter to generate pFastBac.IE1.B19.Rep transfer vector (FIG. 3B). This vector was used to generate recombinant BEV, AcBIVVBac.IE1.B19.Rep^{Tn7} and then tested in polyclonal Sf cell lines encoding hFVIIIco6XTEN with symmetric B19 ITRs. The results showed that the AcMNPV-constitutive ie1-driven B19.Rep was able to rescue the ceDNA vector from both the cell lines tested in comparison with the polyhedrin-driven Rep. This data suggests that the stoiochiometric expression of Rep protein is critical for ceDNA production in Sf9 cells as observed earlier for rAAV production.

### GPV. Rep:

The coding sequence of GPV Rep was obtained from GPV strain B (GenBank accession no. GPU25749). Unlike B19, GPV.Rep produces spliced proteins (Rep78 and Rep52) from a single mRNA transcript, and to achieve the stoichiometric expression of both proteins, the Rep78 coding sequence was genetically modified that would allow the expression of Rep78 and Rep52 polypeptides from a single mRNA species via leaky ribosomal scanning mechanism. The AUG initiation codon of the Rep78 open reading frame (orf), the adjacent proline codon, and twelve downstream AUG triplets occurring before the start codon of the Rep52 orf was altered via gene synthesis. The Rep78 initiation codon and proximal flanking nucleotides were mutated to an inefficient translation initiation signal composed of a CUG triplet presented in the context of a Kozak consensus sequence. AUG triplets occurring between the initiation codon of the Rep78 orf and the AUG initiation codon of the Rep52 orf were altered to bear either a silent mutation (in the case of out-of-frame AUG codons), or to encode a conservative amino acid substitution (in the case of in-frame AUG codons). Finally, the modified GPV.Rep coding sequence was synthesized through GenScript® (SEQ ID NO:12) (FIG. 3C) and the synthetic DNA was cloned into pFastBac1 (Invitrogen) vector under the AcMNPV Polyhedrin promoter to generate pFastBac.Polh.GPV.Rep transfer vector (FIG. 3D). This vector was then transformed into BIVVBac^{DH10B} E. coli to produce a recombinant BEV, AcBIVVBac.Polh.GPV.Rep^{Tn7}, as described below.

### AAV2. Rep:

The coding sequence of AAV2 Rep was obtained from the AAV2 genome (GenBank accession no. NC_001401). Unlike B19.Rep, AAV2.Rep also produces spliced proteins (Rep78 and Rep52) from a single mRNA transcript, and to achieve the stoichiometric expression of both proteins, the Rep78 coding sequence was genetically modified that would allow the expression of Rep78 and Rep52 polypeptides from a single mRNA transcript via leaky ribosomal scanning mechanism, as described earlier (Smith et al., 2009) (FIG. 3E). Modified AAV2.Rep coding sequence was synthesized through GenScript® (SEQ ID NO:13) (FIG. 3E) and the synthetic DNA was cloned into pFastBac1 (Invitrogen) vector under the AcMNPV Polyhedrin promoter to generate pFastBac.Polh.AAV2.Rep transfer vector (FIG. 3F). This vector was then transformed into BIVVBac^{DH10B} E. coli to produce a recombinant BEV, AcBIVVBac.Polh.AAV2.Rep^{Tn7}, as described below.

### Example 4: Human FVIIIco6XTEN Expression Constructs

Human FVIIIco6XTEN Expression Constructs with Non-AAV or AAV ITRs:

The genetic constructs comprising a codon-optimized human FVIII with XTEN 144 peptide (FVIIIco6XTEN) under the liver-specific TTPp promoter, the Woodchuck Posttranscriptional Regulatory Element (WPRE), the Bovine Growth Hormone Polyadenylation (bGHpA) signal, and flanking truncated ITRs of non-AAV or AAV are described in PCT Publication No. WO2019032898A1. Here, these constructs were tested in parallel with the new constructs made by replacing either 5′ or 3′ ITR with the wildtype sequences of non-AAV or AAV (Table 2). These new constructs were grown and maintained in E. coli strain PMC103 containing deleted sbcC gene, which encodes an exonuclease that recognizes and eliminates the cruciform DNA structures. The PMC103 E. coli was able to support the growth of hFVIIIco6XTEN constructs with symmetric or asymmetric ITRs of non-AAV or AAV after optimizing the temperature and the growth conditions of bacterial cultures. All new constructs were selected on ampicillin resistance plate and screened by the restriction enzyme mapping to determine the correct genetic structures. The hFVIIIco6XTEN expression constructs were then used for making Cre-LoxP donor vectors.

TABLE 2
Human FVIIIco6XTEN Expression Constructs

| | Symmetric/ | | |
|---|---|---|---|
| | Asymmetric | 5′ ITR | 3′ ITR |
| Construct-1 | B19 symmetric | B19Δ135 | B19Δ135 |
| | truncated | (SEQ ID NO: 1) | (SEQ ID NO: 1) |
| Construct-2 | B19 symmetric | B19.WT | B19.WT |
| | wildtype | (SEQ ID NO: 2) | (SEQ ID NO: 2) |
| Construct-3 | GPV symmetric | GPVΔ162 | GPVΔ162 |
| | truncated | (SEQ ID NO: 3) | (SEQ ID NO: 3) |
| Construct-4 | GPV symmetric | GPV.WT | GPV.WT |
| | wildtype | (SEQ ID NO: 4) | (SEQ ID NO: 4) |
| Construct-5 | GPV | GPVΔ162 | GPV.WT |
| | asymmetric | (SEQ ID NO: 3) | (SEQ ID NO: 4) |
| Construct-6 | AAV2 | AAV2.WT | AAV2.WT |
| | symmetric | (SEQ ID NO: 5) | (SEQ ID NO: 6) |
| | wildtype | | |
| Construct-7 | AAV2 | AAV2Δ15Δ11 | AAV2Δ15 |
| | asymmetric1 | (SEQ ID NO: 8) | (SEQ ID NO: 7) |
| Construct-8 | AAV2 | AAV2.WT | AAV2Δ15 |
| | asymmetric2 | (SEQ ID NO: 5) | (SEQ ID NO: 7) |
| Construct-9 | AAV2 | AAV2Δ15Δ11 | AAV2.WT |
| | asymmetric3 | (SEQ ID NO: 8) | (SEQ ID NO: 6) |

Human FVIIIco6XTEN Cre-LoxP Donor Vectors with Non-AAV or AAV ITRs:

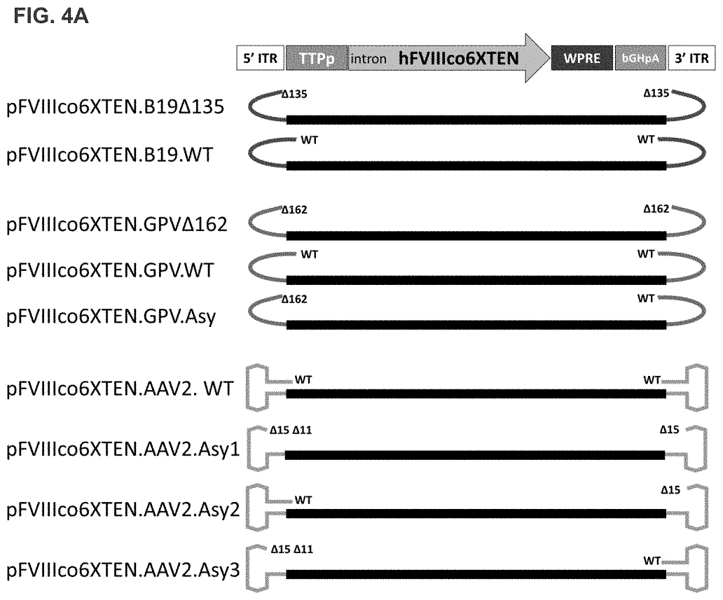

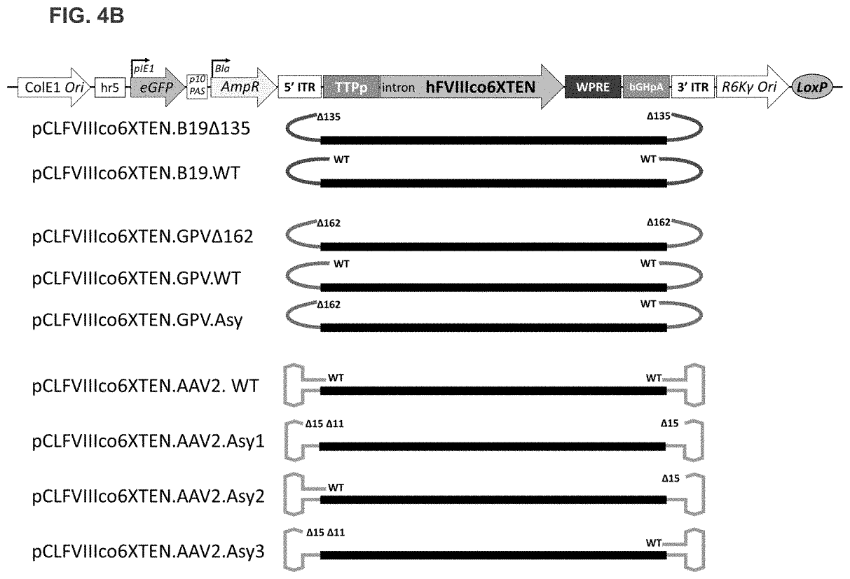

The hFVIIIco6XTEN expression cassette from the constructs-1, -3, and -7 (Table 2) was excised with PstI enzyme and cloned at the same site in Cre-LoxP donor vector. The resulting constructs were transformed into the NEB® Stable Competent E. coli strain (New England Biolabs) and the ampicillin resistance clones were screened by the restriction enzyme mapping. Correct clones were designated with prefix “pCL” for “Plasmid Cre-LoxP” in pCLDV-1, -3, and -7 (Table 2) (FIG. 4B). Similarly, to generate other donor vectors, the hFVIIIco6XTEN expression cassette from the contructs-2, -4, -5, -6, -8, and -9 was excised PstI and/or PvuII enzymes and cloned at the same sites in Cre-LoxP donor vector. These constructs were transformed into the PMC103 E. coli strain due to long palindromic repeats of the wildtype ITR sequences and the ampicillin resistance clones were screened by the restriction enzyme mapping. The resulting Cre-LoxP donor vectors, pCLDV-2, -4, -5, -6, -8, and -9 were then inserted at the LoxP recombination site into the “BIVVBac” bacmid encoding either B19, GPV, or AAV2 Rep at the Tn7 site, respectively (Table 3). Sequences of the ITRs in Table 3 are set forth in Table 1.

TABLE 3
Human FVIIIco6XTEN Cre-LoxP Donor Vectors

| | Cre-LoxP donor vector | 5′ ITR | 3′ ITR |
|---|---|---|---|
| pCLDV-1 | pCL.hFVIIIco6XTEN.B19Δ135 | B19Δ135 | B19Δ135 |
| pCLDV-2 | pCL.hFVIIIco6XTEN.B19.WT | B19.WT | B19.WT |
| pCLDV-3 | pCL.hFVIIIco6XTEN.GPVΔ135 | GPVΔ162 | GPVΔ162 |
| pCLDV-4 | pCL.hFVIHco6XTEN.GPV.WT | GPV.WT | GPV.WT |
| pCLDV-5 | pCL.hFVIIIco6XTEN.GPV.Asy | GPVΔ162 | GPV.WT |
| pCLDV-6 | pCL.hFVIIIco6XTEN.AAV2.WT | AAV2.WT | AAV2. |
| | | | WT |
| pCLDV-7 | pCL.hFVIIIco6XTEN.AAV2.Asy1 | AAV2Δ15Δ11 | AAV2Δ15 |
| pCLDV-8 | pCL.hFVIIIco6XTEN.AAV2.Asy2 | AAV2.WT | AAV2Δ15 |
| pCLDV-9 | pCL.hFVIIIco6XTEN.AAV2.Asy3 | AAV2Δ15Δ11 | AAV2. |
| | | | WT |

### Example 5: Replication (Rep) Baculovirus Expression Vectors (BEVs)

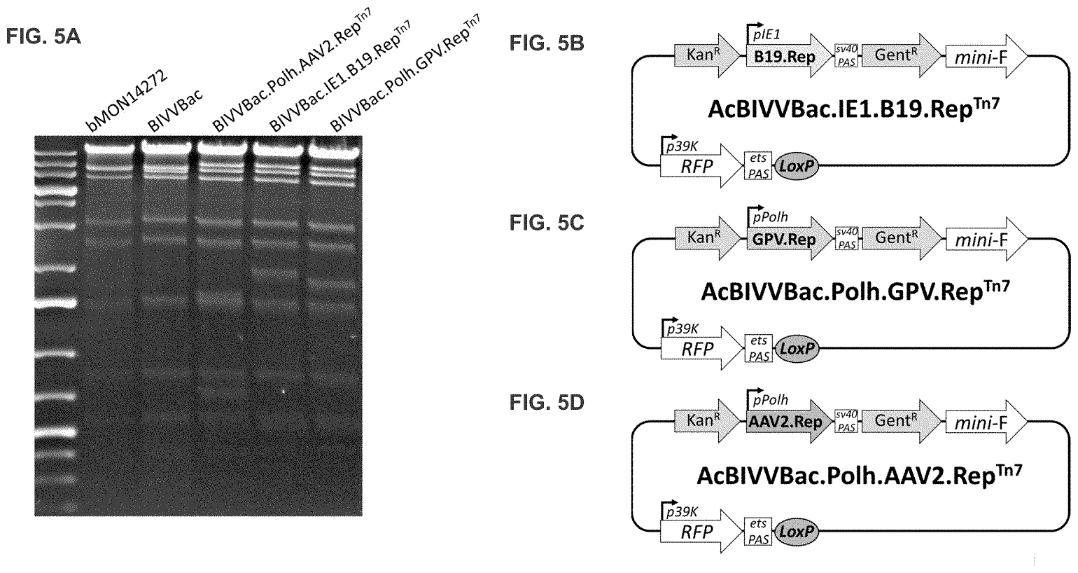

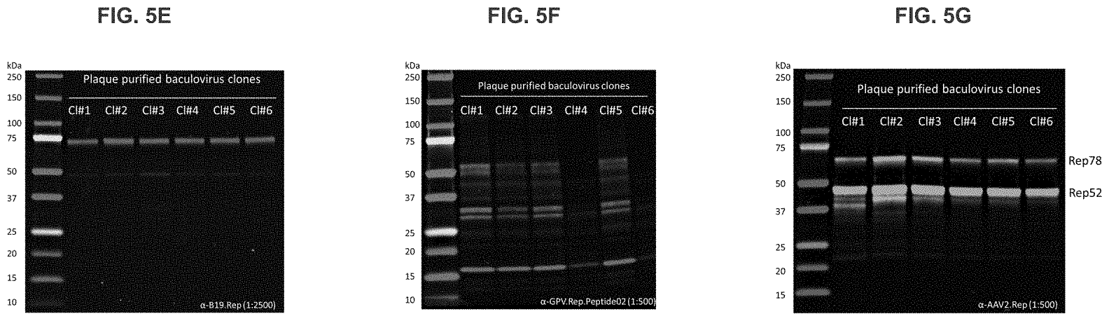

Replication of AAV is dependent on the nonstructural (Replication) proteins; Rep78, Rep68, Rep52, and Rep40. There are several activities of AAV Rep78 and Rep68 have been characterized that are involved in viral DNA replication, including binding to the AAV ITR, sequence- and strand-specific endonuclease activity, and ATP-dependent DNA helicase activity. Either Rep68 or Rep78 binds to the specific region (Rep Binding Site) within the ITR and nicks one strand of the duplex replication intermediate at a unique site called Terminal Resolution Site (TRS). The endonuclease reaction results in covalent attachment of Rep to the newly generated 5′ end. The 3′ end of the nick serves as a primer for extension of the ITR. The helicase activity of the covalently attached Rep molecule may unwind the secondary structure of the ITR. The process of terminal resolution provides the means for the restoration of the termini and generation of progeny viral genomes. Thus, Rep is essential for the ITR-mediated vector production in eukaryotic cells and to ‘rescue’ an ITR-flanked hFVIIIco6XTEN vector genome from Sf9 cells, we generated recombinant BEVs encoding non-AAV or AAV Rep. In order to generate these BEVs, first, BIVVBac^{DH10B} E. coli (FIG. 1D) were super-transformed with transfer vectors, pFastBac.IE1.B19.Rep (FIG. 3B), pFastBac.Polh.GPV.Rep (FIG. 3D) and pFastBac.Polh.AAV2.Rep (FIG. 3F) and then, the transformants were selected on kanamycin, gentamycin, X-Gal, and IPTG. The site-specific transposition of the Rep expression cassette and gentamycin resistance gene at the mini-attTn7 insertion site in BIVVBac disrupted LacZα (fused in-frame with mini-attTn7) and resulted in white E. coli colonies on X-Gal-mediated dual antibiotic selection. Thus, recombinant bacmid DNAs were isolated from white E. coli colonies by alkaline lysis miniprep and digested with restriction enzymes to determine the correct genetic structures. The results of restriction enzyme mapping showed expected fragments for each recombinant bacmid suggesting the site-specific transposition of Rep in the Polyhedrin locus of BIVVBac (FIG. 5A). Further confirmation was obtained by PCR amplifying regions spanning across the expected insertion site using primers internal and external to the transfer plasmid and sequencing the resulting amplimers.

The correct recombinant bacmids encoding B19.Rep, GPV.Rep, or AAV2.Rep were transfected in Sf9 cells using Cellfectin® (Invitrogen) transfection reagent, according to the manufacturer's instructions. At 4-5 days post-transfection, the progeny baculovirus was harvested and plaque purified in Sf9 cells, as described (Jarvis, 2014). Six plaque purified RFP+ clones of each recombinant BEV, AcBIVVBac.IE1.B19.Rep^{Tn7} (FIG. 5B), AcBIVVBac.Polh.GPV.Rep^{Tn7} (FIG. 5C), and AcBIVVBac.Polh.AAV2.Rep^{Tn7} (FIG. 5D) was amplified to P1 (Passage 1) in Sf9 cells seeded at 0.5×10⁶ per mL in T25 flasks in ESF-921 medium supplemented with 10% heat-inactivated Fetal Bovine Serum (FBS). At 4-5 days post-infection, the P1 virus was harvested by low-speed centrifugation and the infected cell pellets were tested for B19.REP, GPV.REP or AAV2.REP detection by immunoblotting using anti-B19 NS1 (MyBioSource, Inc.), anti-GPV.REP (GenScript®), and anti-AAV2.REP 303.9 (American Research Products Inc.) monoclonal or polyclonal antibody, respectively. The immunoblot analysis of AcBIVVBac.IE1.IB19.Rep^{Tn7} plaque purified clones (FIG. 5E) showed single species of B19.REP at the predicted mass of 74 kDa suggesting B19.REP expressed from a non-spliced mRNA transcript in Sf9 cells. Interestingly, there was an equivalent level of B19.REP expression observed in all viral clones tested suggesting a stoichiometric level of B19.REP expression under the AcMNPV IE1 promoter in Sf9 cells. Unlike B19.REP, immunoblot analysis of AcBIVVBac.Polh.GPV.Rep^{Tn7} (FIG. 5F) or AcBIVVBac.Polh.AAV2.Rep^{Tn7} (FIG. 5G) plaque purified clones showed two species of REP at the predicted mass of 73 kDa and 49 kDa corresponds to the Rep78 and Rep52 polypeptides suggesting GPV.REP and AAV2.REP expressed from spliced mRNA transcripts in Sf9 cells. There was an equivalent level of Rep78 or Rep52 observed in all viral clones tested suggesting stable GPV.REP and AAV2.REP expression under the AcMNPV Polyhedrin promoter in Sf9 cells. The level of REP78 expression was almost half of the REP52 further suggesting the correct stoichiometric expression of both polypeptides is achieved from a single mRNA transcript by the leaky ribosomal scanning mechanism. The highest REP expressing clone of each BEV was selected for further amplification in Sf9 cells and then, used for infection in stable cell lines encoding hFVIIIco6XTEN flanked by the symmetric or asymmetric ITRs of non-AAV and AAV for ceDNA vector production.

### Example 6: Replication (Rep) and Human FVIIIco6XTEN Baculovirus Expression Vectors (BEVs)

In order to test whether BIVVBac could be used to accommodate multiple transgenes, a family of derivative vectors were generated encoding two transgene expression cassettes: 1) Rep and, 2) hFVIIIco6XTEN flanked by the symmetric or asymmetric ITRs of non-AAV and AAV. These BEVs were produced in two steps. First, the Rep expression cassette was inserted at the mini-attTn7 site in the Polyhedrin locus via Tn7 transposition, as described above. Then, the resulting recombinant bacmids were used for inserting hFVIIIco6XTEN expression cassette at the LoxP site in the EGT locus via in-vitro Cre-LoxP recombination using Cre recombinase (New England Biolabs). In the process, the Cre-LoxP donor vectors (pCLDV-1 to -9) (Table 2) encoding hFVIIIco6XTEN with B19 ITRs, GPV ITRs, and AAV ITRs were inserted in the AcBIVVBac.IE1.619.Rep (FIG. 5B), AcBIVVBac.Polh.GPV.Rep (FIG. 5C), or AcBIVVBac.Polh.AAV2.Rep (FIG. 5D) bacmid, respectively. The recombination reactions were transformed in DH10B E. coli and the transformants were selected on kanamycin, gentamycin, and ampicillin. Triple antibiotic-resistant colonies were screened by the restriction enzyme mapping and/or PCR by amplifying the regions spanning across the expected insertion site using primers internal and external to the transfer plasmid and sequencing the resulting amplimers.

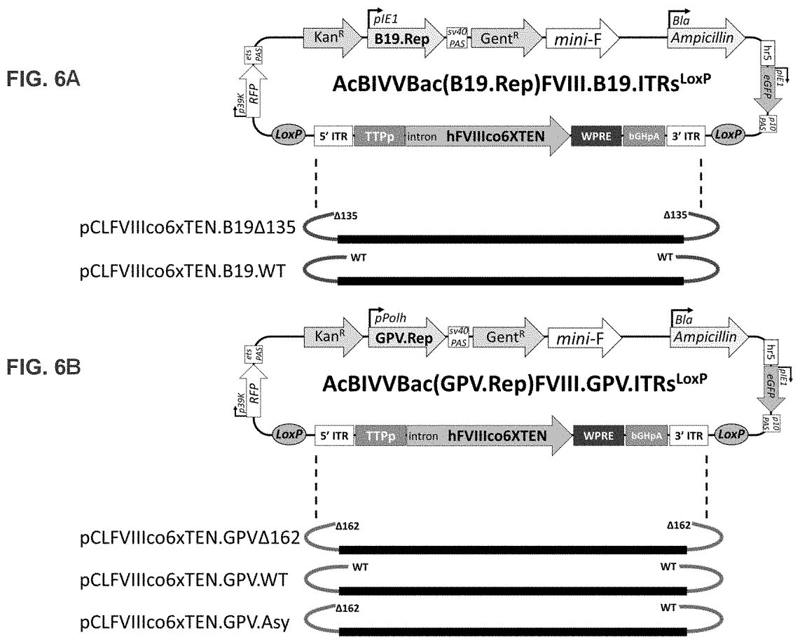

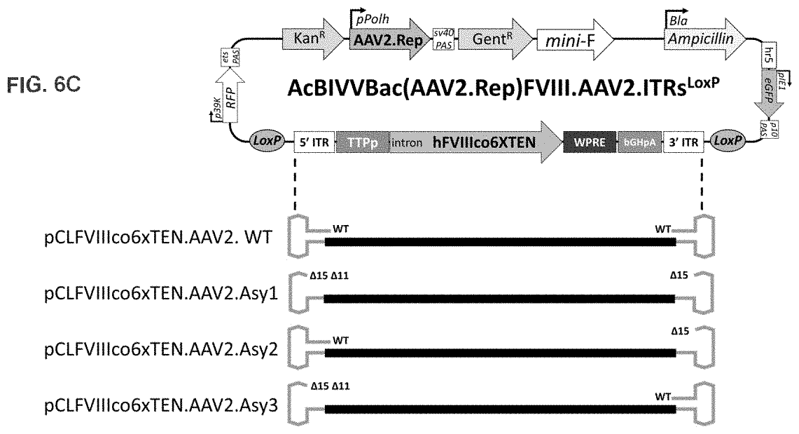

The correct recombinant bacmid encoding both transgene cassettes was maxi prep purified and transfected in Sf9 cells using Cellfectin® (Invitrogen) transfection reagent. At 4-5 days post-transfection, the progeny baculovirus was harvested and plaque purified in Sf9 cells. Six plaque purified RFP+ and GFP+ clones of each recombinant BEV (BEV-1 to -9) (Table 4) (FIGS. 6A, 6B, and 6C) were amplified to P1 (Passage 1) in Sf9 cells seeded at 0.5×10⁶ per mL in T25 flasks in ESF921 medium supplemented with 10% heat-inactivated Fetal Bovine Serum (FBS). At 4-5 days post-infection, all clones showed progression of infection, determined by the number of GFP+ and RFP+ cells, for each recombinant BEV suggesting the virus was able to replicate normally and the insertion of multiple transgenes in the same baculovirus genome had no adverse effect on the progeny virus production. The P1 virus was harvested by low-speed centrifugation and the infected cell pellets were processed either for REP detection by immunoblotting or for ceDNA isolation using PureLink Maxi Prep DNA isolation kit (Invitrogen). Immunoblotting was performed to detect B19.REP, GPV.REP or AAV2.REP, as described above. The results of immunoblotting showed all six clones positive for each REP tested which suggests that the insertion of hFVIIIco6XTEN cassette at the EGT locus had no adverse effect on the immediate-early or polyhedrin-d riven REP expression. Finally, the highest REP expressing clone of each BEV was further amplified to produce working BEV stocks (P2) followed by titering in Sf9 cells. Titrated BEVs were used for infection in Sf9 cells to produce hFVIIIco6XTEN ceDNA vectors, as described below.

TABLE 4
Rep and Human FVIIIco6XTEN BEVs

| | BEVs | ITRs | |
|---|---|---|---|
| BEV-1 | AcBIVVBac(B19.Rep)FVIII.B19Δ135.ITR^{LoxP} | B19 symmetric | FIG. 6A |
| | | truncated | |
| BEV-2 | AcBIVVBac(B19.Rep)FVIII.B19.WT.ITR^{LoxP} | B19 symmetric | |
| | | wildtype | |
| BEV-3 | AcBIVVBac(GPV.Rep)FVIII.GPVΔ162.ITR^{LoxP} | GPV symmetric | FIG. 6B |
| | | truncated | |
| BEV-4 | AcBIVVBac(GPV.Rep)FVIII.GPV.WT.ITR^{LoxP} | GPV symmetric | |
| | | wildtype | |
| BEV-5 | AcBIVVBac(GPV.Rep)FVIII.GPV.Asy.ITR^{LoxP} | GPV asymmetric | |
| BEV-6 | AcBIVVBac(AAV2.Rep)FVIII.AAV2.WT.ITR^{LoxP} | AAV2 symmetric | FIG. 6C |
| | | wildtype | |
| BEV-7 | AcBIVVBac(AAV2.Rep)FVIII.AAV2.Asy1.ITR^{LoxP} | AAV2 | |
| | | asymmetric1 | |
| BEV-8 | AcBIVVBac(AAV2.Rep)FVIII.AAV2.Asy2.ITR^{LoxP} | AAV2 | |
| | | asymmetric2 | |
| BEV-9 | AcBIVVBac(AAV2.Rep)FVIII.AAV2.Asy3.ITR^{LoxP} | AAV2 | |
| | | asymmetrie3 | |

### Example 7: Human FVIIIco6XTEN ceDNA Vector Production from Baculovirus

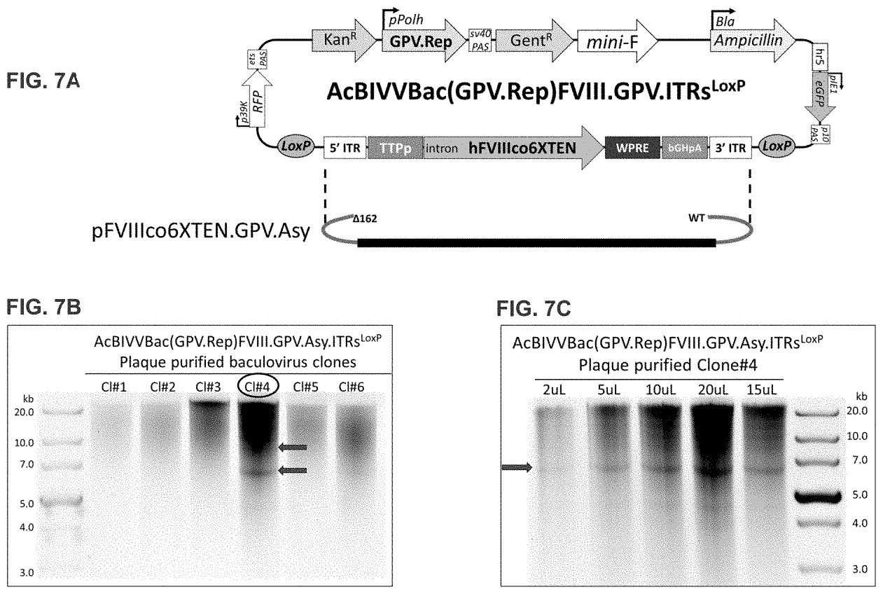

Recombinant BEVs encoding both hFVIIIco6XTEN and Rep genes (Table 4) were tested for ceDNA production. Sf9 cells were infected with the titrated working stock (P2) of each BEV at a multiplicity of infection (MOI) of 3 pfu/cell. Cells were gently tumbled at room temperature for 1.5 h, pelleted at 500×g for 5 min, the supernatant was aspirated, and the cells were washed once with 10 mL of fresh ESF-921 medium. Cells were suspended into 50 mL of ESF-921 medium and then incubated for 72 h at 28° C. in a shaker incubator. At 72 h post-infection, infected cells were harvested, and the pellets were washed once with 1×PBS to remove residual baculoviral particles and/or culture medium. Then, the ceDNA vectors were isolated by the PureLink Maxi Prep DNA isolation kit (Invitrogen), according to the manufacturer's instructions. Elution fractions were analyzed by 0.8 to 1.2% agarose gel electrophoresis to determine the yield and purity of each ceDNA vector. As the first round of analysis, the ceDNA vector isolated from Sf9 cells infected with the BEV-5 (Table 4, FIG. 7A) was tested. All six clones of BEV-5 were tested for ceDNA production. One of the clones showed a DNA band corresponding to the size of the hFVIIIco6XTEN transgene (˜7.0 kb) showing that that the polyhedrin-driven GPV REP was able to rescue asymmetric ITR-flanked hFVIIIco6XTEN vector DNA (FIG. 7B). The sample was run again at different volumes to visualize the banding pattern and confirm the size of ceDNA. Multiple bands of high molecular weights were observed (FIG. 7C). This data proves the concept of ceDNA production from a single recombinant BEV encoding non-AAV Rep as well as ITR-flanked hFVIIIco6XTEN transgene.

### Example 8: Human FVIIIco6XTEN Stable Insect Cell Lines

It was reasoned that the insect cell genome can also be modified to produce DNA therapeutic drug substances following baculovirus infection. In order to achieve this goal, a plasmid encoding neomycin resistance marker under the AcMNPV immediate early (ie1) promoter preceded by the transcriptional enhancer hr5 element and followed by the AcMNPV p10 polyadenylation signal was synthesized from GenScript® (Piscataway, N.J.) (SEQ ID NO:14) (FIG. 8A). This synthetic DNA was co-transfected with the plasmids encoding hFVIIIco6XTEN expression cassette flanked by the symmetric and asymmetric ITRs of non-AAV or AAV (Table 3) in Sf9 cells using Cellfectin® (Invitrogen™). At 24 h post-transfection, cells were visualized under the fluorescence microscope to determine the transfection efficiency and the results showed >80% GFP+ cells suggesting higher transfection efficiency. At 72 h post-transfection, cells were selected with G418 antibiotic (Sigma Aldrich) suspended in complete TNMFH medium (Grace's Insect Medium supplemented with 10% FBS+0.1% Pluronic F68) at 1.0 mg/mL final concentration. After about a week of selection, there were ˜50% of transformed cells recovered which suggests that the neomycin resistant marker was stably integrated into this cell population. The survivor cells were taken off the selection media and fed with a fresh complete TNMFH medium until confluence growth. The confluent cells were progressively expanded as an adherent culture into larger culture vessels as they continue to divide. Later, each cell line was adapted to the suspension culture by growth in shake flasks for one passage in complete TNMFH and one passage in ESF-921 medium supplemented with 10% FBS. Finally, each cell line was adapted to serum-free ESF-921 in shake flasks as suspension cultures. These shake flask cultures were routinely maintained in serum-free ESF-921 medium with passages every four days and cell growth was monitored. Each cell line is designated according to the hFVIIIco6XTEN construct inserted and prefixed with “Sf” (FIG. 8B) (Table 5).

TABLE 5
Human FVIIIco6XTEN Stable Insect Cell Lines

| | Human FVIIIco6XTEN Cell line | 5′ ITR | 3′ ITR |
|---|---|---|---|
| Cell line-1 | Sf.hFVIIIco6XTEN.B19Δ135 | B19Δ135 | B19Δ135 |
| Cell line-2 | Sf.hFVIIIco6XTEN.B19.WT | B19.WT | B19.WT |
| Cell line-3 | Sf.hFVIIIco6XTEN.GPVΔ135 | GPVΔ162 | GPVΔ162 |
| Cell line-4 | Sf.hFVIIIco6XTEN.GPV.WT | GPV.WT | GPV.WT |
| Cell line-5 | Sf.hFVIIIco6XTEN.GPV.Asy | GPVΔ162 | GPV.WT |
| Cell line-6 | Sf.hFVIIIco6XTEN.AAV2.WT | AAV2.WT | AAV2.WT |
| Cell line-7 | Sf.hFVIIIco6XTEN.AAV2.Asy1 | AAV2Δ15Δ11 | AAV2Δ15 |
| Cell line-8 | Sf.hFVIIIco6XTEN.AAV2.Asy2 | AAV2.WT | AAV2Δ15 |
| Cell line-9 | Sf.hFVIIIco6XTEN.AAV2.Asy3 | AAV2Δ15Δ11 | AAV2.WT |

### Example 9: Human FVIIIco6XTEN ceDNA Vector Production from Stable Cell Lines

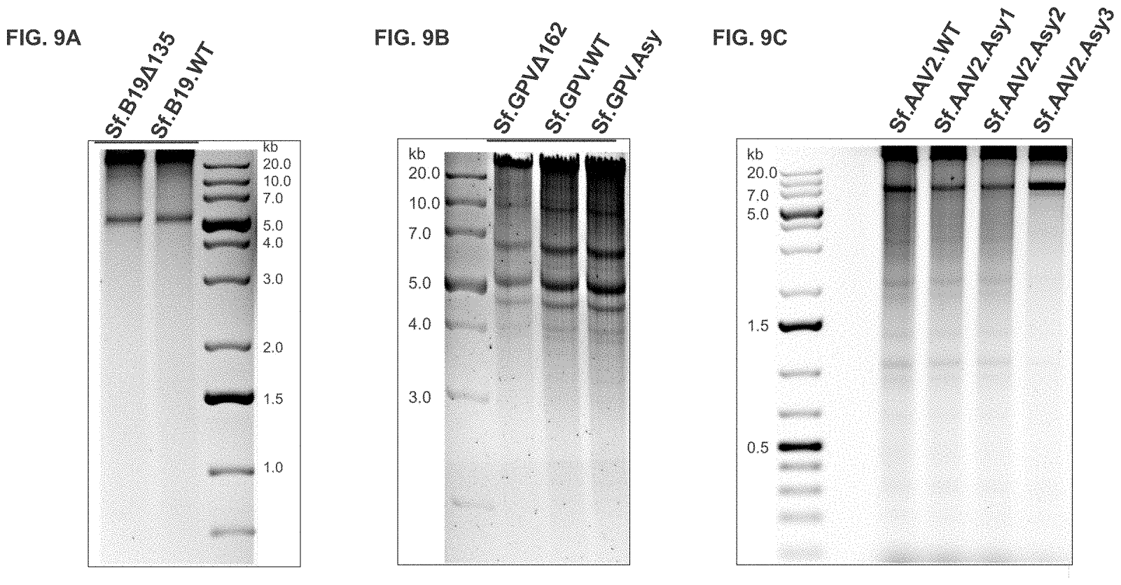

In order to test the stable cell line approach, a polyclonal population of each cell line encoding hFVIIIco6XTEN expression cassette with symmetric or asymmetric ITRs of non-AAV and AAV (Table 4) was used. Cells were infected with the titrated working stock (P2) of each recombinant BEV encoding Rep (FIG. 5B-D) at a multiplicity of infection (MOI) of 3 pfu/cell. Cells were gently tumbled at room temperature for 1.5 h, pelleted at 500×g for 5 min, the supernatant was aspirated, and the cells were washed once with 10 mL of fresh ESF-921 medium. Finally, cells were suspended into 50 mL of ESF-921 medium and then incubated for 72 h at 28° C. in a shaker incubator. At 72 h post-infection, infected cells were harvested, and the pellets were washed once with 1×PBS to remove residual baculoviral particles and/or culture medium. Then, the ceDNA vectors were isolated by the PureLink Maxi Prep DNA isolation kit (Invitrogen), according to the manufacturer's instructions. Elution fractions were analyzed by 0.8 to 1.2% agarose gel electrophoresis to determine the yield and purity of each ceDNA vector. The results showed DNA bands correspond to the size of hFVIIIco6XTEN (˜7.0 kb) with varying degrees of intensity as well as the high molecular weight bands, as observed earlier (FIG. 9A-C). Interestingly, all the cell lines tested showed DNA bands correspond to the size of hFVIIIco6XTEN expression cassette irrespective of the flanking symmetric and/or asymmetric ITRs. The GPV cell lines-3, -4, and -5 showed multiple bands of various sizes in comparison with the B19 or AAV2 lines.

### Example 10: Human FVIIIco6XTEN Expression from ceDNA

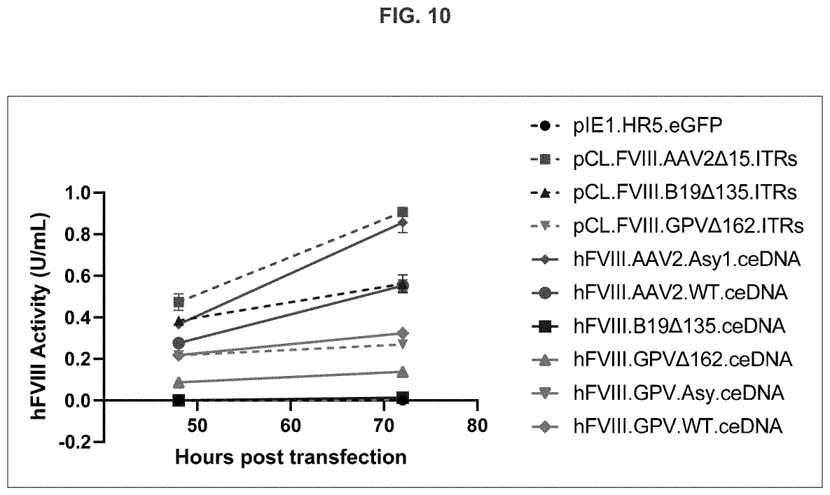

To determine whether ceDNA could be used as a non-viral gene therapy vector for treating Hemophilia A patients, an in vitro assay was performed to test human FVIII expression from ceDNA in hepatocyte-derived carcinoma human Huh7 cells. First, Huh7 cells were seeded at 5×10⁵ cells/mL in 24-well plates in the modified IMEM medium (Gibco™) supplemented with 10% FBS and 0.1% antibiotic mixtures (Invitrogen™) and then, incubated at 37° C. incubator with CO₂ (5%) for 48 h to achieve the monolayer due to static cell growth. This growth condition was achieved to mimic the hFVIIIco6XTEN expression in the nucleus of non-diving liver cells in vivo. The monolayer was transfected with ˜1 ug of gel-purified ceDNAs correspond to the size of the hFVIIIco6XTEN expression cassette using Lipofectamine 3000 (Invitrogen™), according to the manufacturer's instructions. Plasmid DNAs encoding hFVIIIco6XTEN flanked by the symmetric truncated ITRs were used as controls (pCLDV-1, -3, and -7, Table 3). At 18 h post-transfection, the transfection mixture was aspirated, and cells were fed with fresh IMEM medium supplemented with 10% FBS and 0.1% antibiotic mixtures. Cell-free supernatant was aliquoted from each treatment at 48 h and 72 h post-feeding and the secreted hFVIIIco6XTEN was measured by the Chromogenix Coatest® SP Factor VIII chromogenic assay, according to the manufacturer's instructions. The FVIII activity was normalized with the standard and plotted as U/mL in FIG. 10. The assay results showed increased levels of hFVIII expression over the time for all samples tested except hFVII.B19Δ135.ceDNA. This data suggests that the FVIII was expressed from the ceDNA vectors that were potentially transfected in the nucleus of a non-dividing monolayer of Huh7 cells. ceDNAs obtained from GPV cell lines-3, -4, and -5 showed low level of FVIII expression in comparison with AAV lines-6 and -8 which suggests that the GPV ITRs most likely get truncated while integrating into the cell genome, as described above. Surprisingly, ceDNAs obtained from B19 cell lines-1 and -2 showed no detectable hFVIIIco6XTEN expression while it was detectable in the plasmid DNA (pCLDV-1, Table 3) used as a control. Overall, the ceDNA vectors of GPV and AAV showed persistent hFVIII expression at the physiological levels in Huh7 cells which, suggests that the ceDNA could be used as a non-viral gene therapy vector (FIG. 10). Individual cell lines are described in Table 5.

### Example 11: Human FVIIIco6XTEN ceDNA Vector Production by Transient Transfection

Transient gene expression system is one of the most important technologies for performing functional analysis in the baculovirus-insect cell system. This system was developed to express foreign genes under the control of baculoviral promoter in transient expression plasmids. Furthermore, this system offers shorter turn-around on vector constructions and protein synthesis, avoids challenges associated with viral infection and cell lysis, and provides a robust means to observe the cellular trafficking of proteins. Baculovirus gene promoters in general are divided into immediate early, early, late, and very late promoters according to their onset of transcription in the infection cycle. Among these, only immediate-early (ie) gene promoters are recognized by the host RNA-polymerase II and are independent of viral transcriptional factors, making them suitable for baculovirus-free heterologous protein expression in insect cells. The most widely and commercially available transient gene expression system is developed based on the immediate-early (ie) gene promoters of Orgyia psuedotsugata multiple nucleopolyhedrovirus (OpMNPV). In comparison with the other immediate-early (ie) gene promoters, OpIE2 promoter of OpMNPV has been shown to provide strong activity for heterologous protein expression in Sf cells (Bleckmann et al., 2016). More recently, a baculovirus-free system based on the polyethylenimine (PEI)-mediated transient gene expression under the control of OpIE2 promoter was also described (Puente-Massaguer et al., 2020).

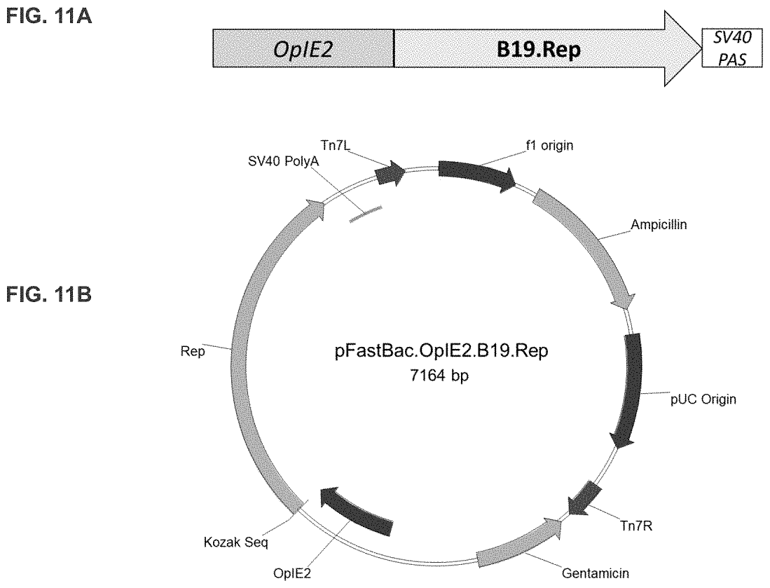

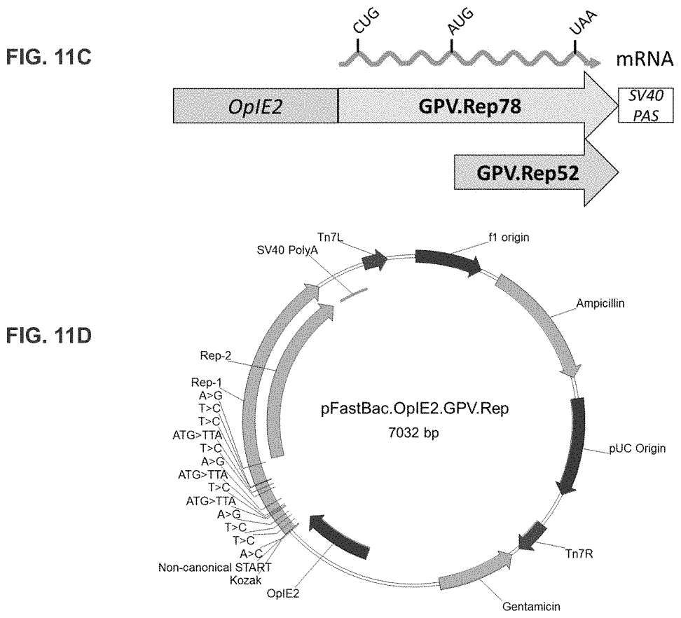

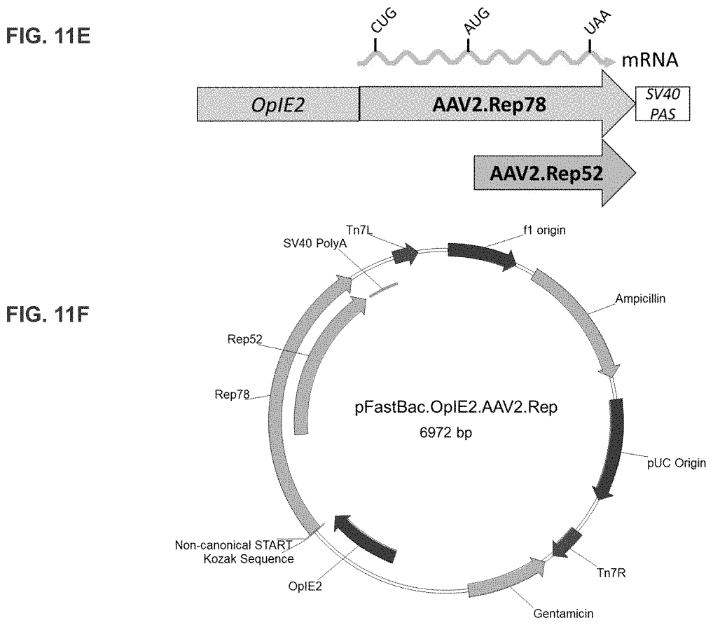

Thus, it was hypothesized that the transient expression of Rep protein could “rescue” the human FVIIIco6XTEN ceDNA vector from the stable cell lines. To test this hypothesis, the PEI-mediated transient Rep protein expression under the OpMNPV immediate-early (OpIE2) promoter for the baculovirus-free ceDNA production in the stable cell lines was utilized. The OpIE2 promoter sequence from OpMNPV genome (GenBank accession no. NC_001875.2) was synthesized through GenScript® (Piscataway, N.J.) (SEQ ID NO:15). The synthetic promoter sequence was cloned to replace the polyhedrin or immediate-early promoter in the Rep protein expression constructs (FIG. 11A, 11C, 11E). The resulting transient expression plasmids, pFastBac.OpIE2.B19.Rep (FIG. 11B), pFastBac.OpIE2.GPV.Rep (FIG. 11D), and pFastBac.OpIE2.AAV2.Rep (FIG. 11F) will be used to transfect stable cell lines encoding human FVIIIco6XTEN expression cassette with symmetric or asymmetric ITRs of non-AAV and AAV (Table 5).

The stable cell lines encoding human FVIIIco6XTEN expression cassette with symmetric or asymmetric ITRs of non-AAV and AAV (Table 5) will be super-transformed with the transient expression plasmids encoding B19.Rep, GPV.Rep or AAV2.Rep under the OpMNPV immediate-early (OpIE2) promoter (FIG. 11) and a plamid encoding puromycin resistance marker under the AcMNPV immediate early (ie1) promoter preceded by the transcriptional enhancer hr5 element and followed by the AcMNPV p10 polyadenylation signal to generate new set of stable cell lines. The transformed cells will be selected with puromycin and amplified to a shake flask culture, as described in Example 8. These cell lines will be used as a producer cell lines for the baculovirus-free human FVIIIco6XTEN ceDNA vector production.

### Example 12: Modified FVIIIXTEN Expression Cassette

It was hypothesized that the transgene expression level can be increased by codon-optimizing cDNA for the targeted hosts. The physiological levels of FVIII expression from V1.0 FVIIIco6XTEN expression cassette have been demonstrated in previous studies as described in U.S. Publication No. 20190185543. However, to further improve the target specificity and reduce the immunogenicity, the FVIIIXTEN cDNA was codon-optimized with CpG repeats depleted to escape innate immune response raised against the DNA vector encoding FVIIIXTEN expression cassette with parvoviral ITRs. The modified V2.0 FVIIIXTEN expression cassette was generated which comprises a B-domain deleted (BDD) codon-optimized human Factor VIII (BDDcoFVIII) fused with XTEN 144 peptide (FVIIIXTEN) under the regulation of liver-specific modified mouse transthyretin (mTTR) promoter (mTTR482) with enhancer element (A1MB2), hybrid synthetic intron (Chimeric Intron), the Woodchuck Posttranscriptional Regulatory Element (WPRE), and the Bovine Growth Hormone Polyadenylation (bGHpA) signal. The V2.0 FVIIIXTEN expression cassette comprises the nucleotide sequence of SEQ ID NO: 19.

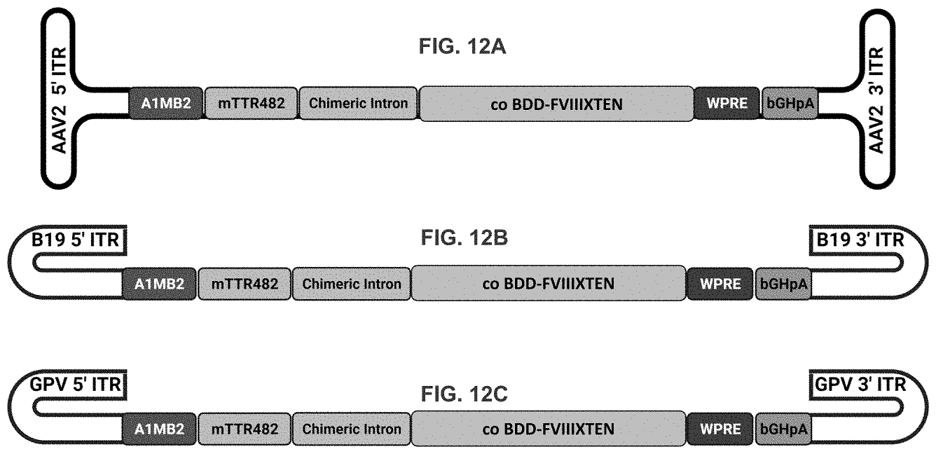

Initial in vivo efficacy studies showed significant improvement in FVIII activity in comparison with the V1.0 FVIIIXTEN expression cassette (data not shown). Therefore, the V2.0 FVIIIXTEN expression cassette was used to generate recombinant BIVVBac bacmids, as described above (Example 6). Then, engineered parvoviral ITRs including AAV2 WT (FIG. 12A), B19 WT or Minimal (SEQ ID NO. 16) (FIG. 12B), and GPVΔ120 (SEQ ID NO. 17) or GPVΔ186 (SEQ ID NO. 18) (FIG. 12C) were inserted into the V2.0 FVIIIXTEN expression cassette (SEQ ID NO: 19) and tested for FVIIIXTEN ceDNA vector production in the baculovirus system using three different approaches, as described below.

### Example 13: Approaches of FVIIIXTEN ceDNA Production

In the baculovirus-insect cell system, recombinant BEV delivers the gene of interest under a strong promoter and provides transcriptional complex essential for the virus replication in insect cells. This system provides the flexibility of inserting transgene of interest in the baculovirus genome and/or insect cell genome in a form of stable cell line. These advantages of the baculovirus system were leveraged in the design of three different approaches of ceDNA production to provide the flexibility of platform selection according to the ease of scalability.

OneBAC:

To investigate the use of a OneBac approach for transgene expression, the BIVVBac bacmid was used. BIVVBac is designed to accept multiple transgene insertions by two different mechanisms at two different sites within the baculovirus genome, as described in Example 1. The optimized FVIIIXTEN expression cassette (SEQ ID NO: 19) was inserted with parvoviral ITRs at the mini-attTn7 site in the Polyhedrin locus in BIVVBac through Tn7 transposition and in the same backbone, the ITR-specific Replication (Rep) gene expression cassette was inserted at the LoxP site in the EGT locus through Cre-LoxP recombination, as described above (Example 6). The recombinant BEV was then generated and used for infection in Sf9 cells to produce FVIIIXTEN ceDNA, as depicted in FIG. 13A. Different promoters for controlling the Rep expression levels were used to prove the concept of the OneBac approach for ceDNA production, as described below.

TwoBAC:

To investigate the use of a TwoBac approach for transgene expression, the optimized FVIIIXTEN expression cassette was inserted with parvoviral ITRs and/or the ITR-specific Replication (Rep) gene expression cassette was inserted at the mini-attTn7 site in the polyhedrin locus in two different BIVVBac bacmids through Tn7 transposition, as described above in Example 5. The recombinant BEVs were then generated and used for co-infection in Sf9 cells to produce FVIIIXTEN ceDNA, as depicted in FIG. 13B. Challenges associated with the TwoBAC approach were investigated using different ratios of multiplicity of infection (MO's) of two baculoviruses and fine-tuning of the Rep expression levels to obtain reproducible ceDNA productivity, as described in the following experiments.

Stable Cell Line:

To investigate the use of a stable cell line approach for transgene expression, a stable cell line was generated with the optimized FVIIIXTEN expression cassette with parvoviral ITRs, as described in Example 8. A recombinant bacmid was also generated by inserting the ITR-specific Replication (Rep) gene expression cassette at the mini-attTn7 site in the polyhedrin locus in BIVVBac bacmid through Tn7 transposition, as described above in Example 5. The recombinant Rep.BEV was then generated and used for infection in the FVIIIXTEN stable cell line to produce FVIIIXTEN ceDNA, as depicted in FIG. 13C. Challenges associated with the stable cell line approach were investigated by enriching FVIIIXTEN transformers through FACS cell sorting using GFP as a proxy to expedite the process of generating stable cell line, as described in the following experiments.

### Example 14: FVIIIXTEN ceDNA (ceFVIIIXTEN) Vector Production from OneBAC

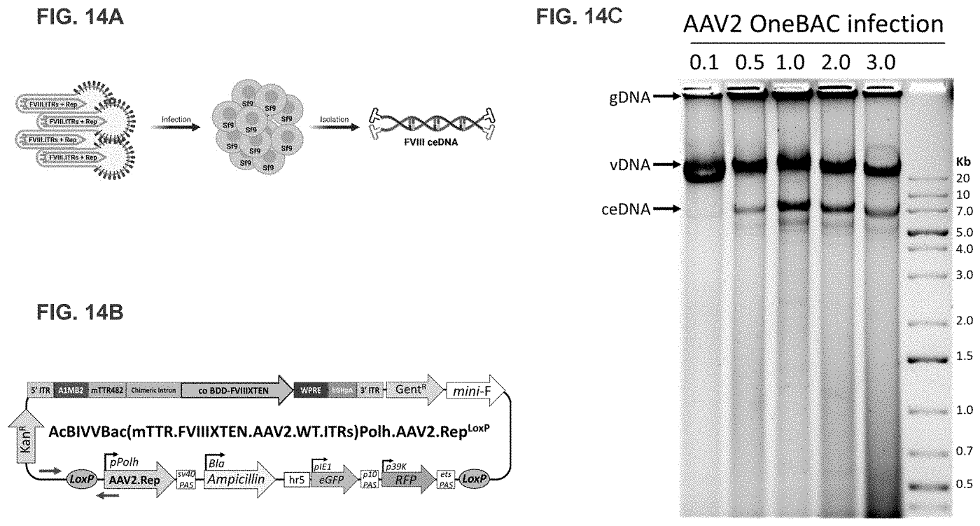

Recombinant BEVs encoding V2.0 FVIIIXTEN with AAV2 WT or B19 WT ITRs and their respective Rep genes under the AcMNPV polyhedrin promoter were tested for FVIIIXTEN ceDNA (ceFVIIIXTEN) production in Sf9 cells using OneBAC approach, as described above. About 2.0×10⁶/mL cells were infected with OneBAC BEVs at an MOI of 0.1, 0.5, 1.0, 2.0, or 3.0 plaque forming units (pfu)/cell (FIG. 14A, 15A). Cells were suspended into 50 mL of serum-free ESF-921 medium and then incubated for 72-96 h or until the viability reached at 60-70% in 28° C. shaker incubator. At ˜96 h post-infection, infected cells were harvested, and the pellets were processed for ceFVIIIXTEN isolation using PureLink Maxi Prep DNA isolation kit (Invitrogen), according to the manufacturer's instructions. Final elution fractions were analyzed on 0.8 to 1.2% agarose gel electrophoresis to determine the ceFVIIIXTEN productivity.

ceFVIIIXTEN AAV2 ITRs

Agarose gel analysis of AAV2 OneBAC (FIG. 14B) showed DNA band corresponding to the size of ceFVIIIXTEN AAV2 ITRs (˜8.5 kb) in all MOIs tested with the highest productivity obtained at an MOI of 1.0 pfu/cell in comparison to other treatments (FIG. 14C). This result was in contrary to early data indicating that the ceFVIIIXTEN obtained from the HBoV1 ITR constructs showed an increase in productivity with increases in viral load (data not shown). Without being bound by theory, this may suggest that the AAV2 Rep has a different mechanism of binding and endonuclease activity at the terminal resolution site of AAV2 WT ITRs for DNA replication in comparison with the HBoV1-NS1 protein, which could be due to the distinct hairpin structures (T-vs. U-shaped) of the respective ITRs.

ceFVIIIXTEN B19 ITRs

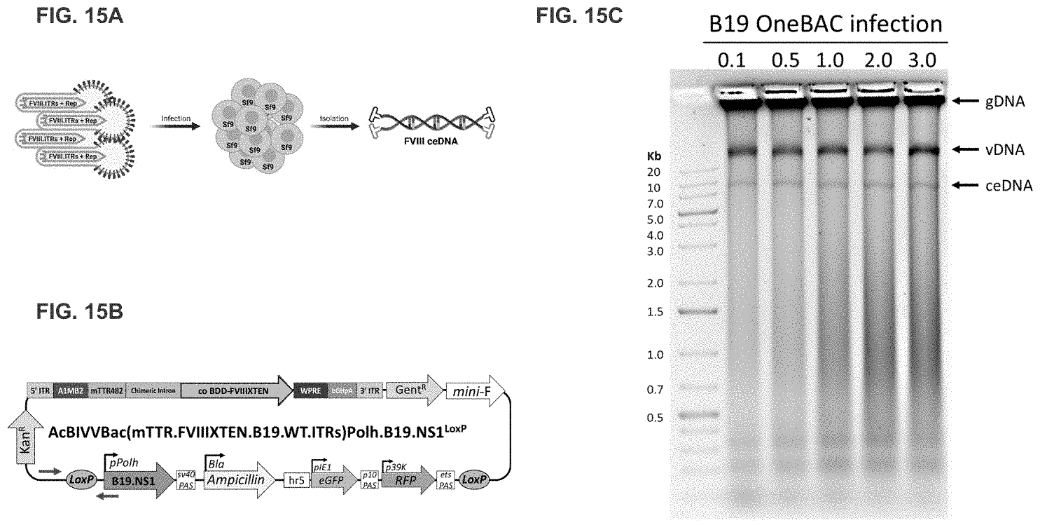

Agarose gel analysis of B19 OneBAC (FIG. 15B) showed a DNA band corresponding to the size of ceFVIIIXTEN B19 ITRs (˜8.5 kb) in all MOIs tested though with low levels of productivity (FIG. 15C). Unlike AAV2 or HBoV1, higher virus load of B19 OneBAC did not improve the ceFVIIIXTEN productivity, potentially due to the higher levels of B19-NS1 expressed at a later stage of infection under the strong (polyhedrin) baculoviral promoter. These results suggest that the combination of full-length (WT) parvoviral ITRs and stoichiometric REP expression may be critical for obtaining higher ceDNA productivity in the baculovirus system.

In conclusion, these experiments have shown that the OneBAC approach does prove the concept of ceDNA production from a single recombinant BEV encoding FVIIIXTEN with parvoviral ITRs and NS1 transgenes. It also shows the feasibility and functionality of multiple transgenes inserted at different loci in a baculovirus shuttle vector (BIVVBac), and its potential use for recombinant AAV vector production in the baculovirus insect cell system.

### Example 15: FVIIIXTEN ceDNA (ceFVIIIXTEN) Vector Production from TwoBAC

To investigate the TwoBAC approach to ceDNA transgene expression, several different conditions of co-infection were tested either by keeping the ratio constant and co-infecting Sf9 cells with TwoBAC at different MOIs or by keeping the MOI constant and co-infecting Sf9 cells with TwoBAC at different ratios. In each case, virus inoculum was not removed, and the cells were incubated until the viability reached at 60-70% in 28° C. shaker incubator. At ˜96 h post-infection, infected cells were harvested, and the pellets were processed for ceFVIIIXTEN isolation using PureLink Maxi Prep DNA isolation kit (Invitrogen), according to the manufacturer's instructions. Final elution fractions were analyzed on 0.8 to 1.2% agarose gel electrophoresis to determine the ceFVIIIXTEN productivity.

ceFVIIIXTEN AAV2 ITRs

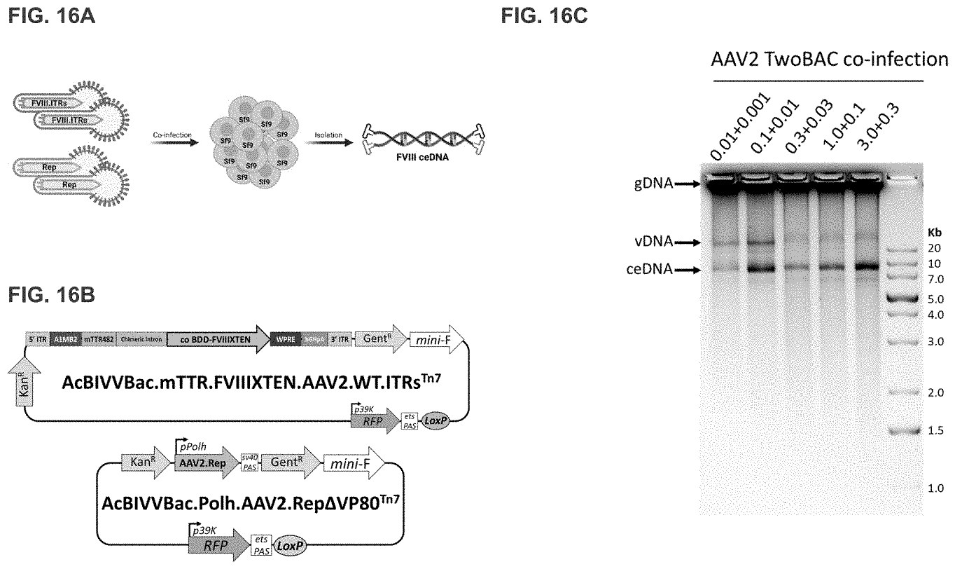

About 2.0×10⁶/mL cells seeded in 50 mL of serum-free ESF-921 medium were co-infected with titrated working stocks (P2) of AcBIVVBac.mTTR.FVIIIXTEN.AAV2.WT. ITRs^{Tn7} BEV at an MOI of 0.01, 0.1, 0.3, 1.0, or 3.0 pfu/cell and AcBIVVBac.Polh.AAV2. RepΔVP80^{Tn7} BEV (previously described in U.S. Patent Application No. 63/069,115) at an MOI of 0.001, 0.01, 0.03, 0.1, or 0.3 pfu/cell for keeping the ratio 1:10 constant, respectively (FIGS. 16A-16B). Agarose gel analysis of AAV2 TwoBAC showed varying degree of ceFVIIIXTEN (ceDNA) productivity in comparison with the contaminating baculoviral DNA (vDNA) at different MOIs of co-infection. Though MOIs of 0.1-0.01 and 3.0-0.3 pfu/cell showed equivalent levels of ceFVIIIXTEN (ceDNA) productivity, the later co-infection MOIs showed lower levels of contaminating vDNA (FIG. 16C).

These results suggest that though AAV2 TwoBAC approach shows equivalent levels of ceFVIIIXTEN productivity to OneBAC, there is a significant difference in the levels of other impurities co-purified with this method of purification. Without being bound by theory, this may further emphasize the importance of stoichiometric AAV2 REP78/REP52 expression for achieving higher ceFVIIIXTEN productivity in Sf9 cells.

ceFVIIIXTEN B19 ITRs

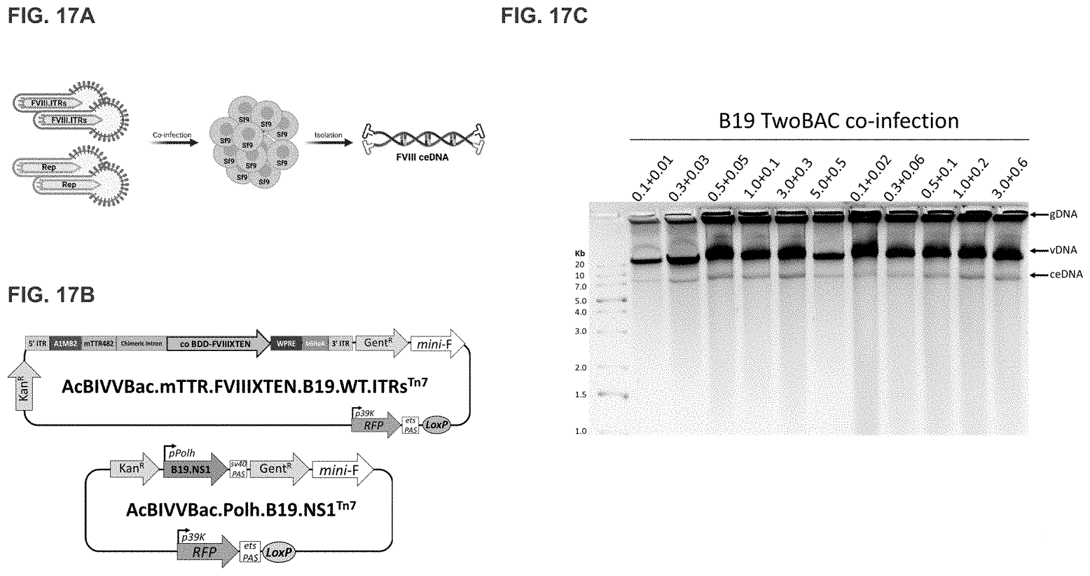

About 2.0×10⁶/mL cells seeded in 50 mL of serum-free ESF-921 medium were co-infected with titrated working stocks (P2) of AcBIVVBac.mTTR.FVIIIXTEN.B19.WT. ITRs^{Tn7} BEV at an MOI of 0.1, 0.3, 0.5, 1.0, 3.0, or 5.0 pfu/cell and AcBIVVBac.Polh.B19-NS1^{Tn7} BEV at an MOI of 0.01, 0.03, 0.05, 0.1, 0.3, or 0.5 pfu/cell for keeping the ratio 1:10 or at an MOI of 0.02, 0.06, 0.1, 0.2, or 0.6 pfu/cell for keeping the ratio 1:5 constant, respectively (FIG. 17A-17B). Unlike AAV2 TwoBAC, agarose gel analysis of B19 TwoBAC showed equivalent levels of ceFVIIIXTEN (ceDNA) and contaminating baculoviral DNA (vDNA) productivity at different MOIs of co-infection (FIG. 17C). Interestingly, none of the conditions tested showed improvement in the ceFVIIIXTEN B19 ITRs productivity like the results obtained with B19 OneBAC.

In conclusion, both the B19 OneBAC and B19 TwoBAC approaches prove the concept of ceDNA production from non-AAV parvoviral ITRs in the baculovirus system.

ceFVIIIXTEN GPV ITRs

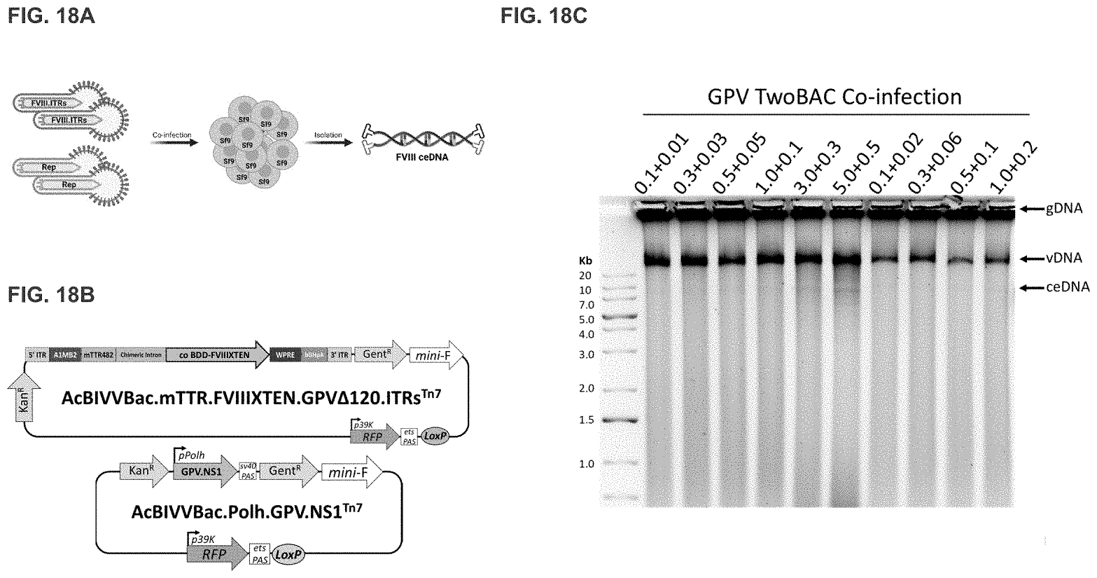

About 2.0×10⁶/mL cells seeded in 50 mL of serum-free ESF-921 medium were co-infected with titrated working stocks (P2) of AcBIVVBac.mTTR.FVIIIXTEN.GPVΔ120. ITRs^{Tn7} BEV at an MOI of 0.1, 0.3, 0.5, 1.0, 3.0, or 5.0 pfu/cell and AcBIVVBac.Polh.GPV-NS1^{Tn7} BEV at an MOI of 0.01, 0.03, 0.05, 0.1, 0.3, or 0.5 pfu/cell for keeping the ratio 1:10 or at an MOI of 0.02, 0.06, 0.1, 0.2 pfu/cell for keeping the ratio 1:5 constant, respectively (FIG. 18A-18B). Unlike AAV2 or B19 TwoBAC, agarose gel analyses of GPV TwoBAC showed no detectable DNA bands corresponding to the size of ceFVIIIXTEN (˜8.5 kb) in all MOIs tested except a faint band observed at an MOI of 5.0-0.5 pfu/cell co-infection (FIG. 18C). The ceFVIIIXTEN productivity obtained with GPV TwoBAC correlates with the GPV OneBAC, where V1.0 FVIIIco6XTEN was used with GPV Asymmetric ITRs, as described above (FIG. 7B, see Example 7).

In both studies, truncated GPV ITRs were tested due to the cloning complexity in obtaining full-length WT GPV ITRs on both ends of the FVIIIXTEN transgene cassettes. Nevertheless, both the GPV OneBAC and GPV TwoBAC approaches prove the concept of ceDNA production and demonstrates the significance of optimum MOI ratio and promoter selection for achieving higher productivity of FVIIIXTEN ceDNA in Sf9 cells.

### Example 16: FVIIIXTEN ceDNA (ceFVIIIXTEN) Vector Production from Stable Cell Line

In order to test the stable cell line approach, a polyclonal population of each cell line made with V2.0 FVIIIXTEN flanked by the AAV2 WT, B19 Minimal, or GPVΔ120 ITRs was seeded at ˜2.0×10⁶/mL in 50 mL of serum-free ESF921 medium and infected with titrated working stock (P2) of ITR-specific REP.BEV at different MOIs, as indicated. In each case, the virus inoculum was not removed, and the cells were incubated for 72-96 h or until the viability reached at 60-70% in 28° C. shaker incubator. At ˜96 h post-infection, infected cells were harvested, and the pellets were processed for ceFVIIIXTEN isolation using PureLink Maxi Prep DNA isolation kit (Invitrogen), according to the manufacturer's instructions. Final elution fractions were analyzed on 0.8 to 1.2% agarose gel electrophoresis to determine the ceFVIIIXTEN productivity.

ceFVIIIXTEN AAV2 ITRs

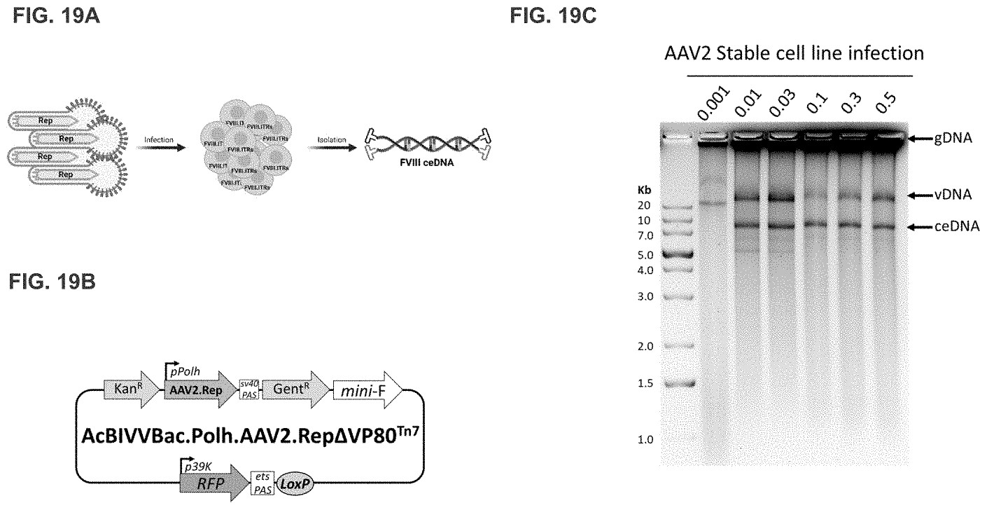

A cell line encoding V2.0 FVIIIXTEN with AAV2 WT ITRs was developed as described earlier (Example 8). Polyclonal Sf.FVIIIXTEN.AAV2.WT.ITRs cell population was then infected with the titrated working stock (P2) of AcBIVVBac.Polh.AAV2.RepΔVP80^{Tn7} BEV (described in U.S. Patent Application No. 63/069,115) at an MOI of 0.001, 0.01, 0.03, 0.1, 0.3, or 0.5 pfu/cell (FIG. 19A, 19B). Agarose gel analysis of ceFVIIIXTEN isolated from the AAV2 stable cell line showed equivalent levels of productivity at all MOIs tested except the lowest MOI of 0.001 pfu/cell, which showed no detectable DNA band corresponding to the size of ceFVIIIXTEN (˜8.5 kb). Interestingly, cells infected with REP.BEV at an MOI of 0.01 and 0.03 pfu/cell showed higher productivity of ceFVIIIXTEN in parallel with the contaminating baculoviral DNA (vDNA) in comparison with the higher MOIs (FIG. 19C). These results suggest that unlike the AAV2 OneBAC or AAV2 TwoBAC approach, the stable cell line approach requires lower virus inoculum to “rescue” stably integrated FVIIIXTEN expression cassette flanked by the AAV2 WT ITRs in Sf9 cell genome.

In conclusion, this study proves the concept of ceDNA production with a cell line stably integrated with a FVIIIXTEN expression cassette flanked with parvoviral ITRs.

ceFVIIIXTEN B19 ITRs

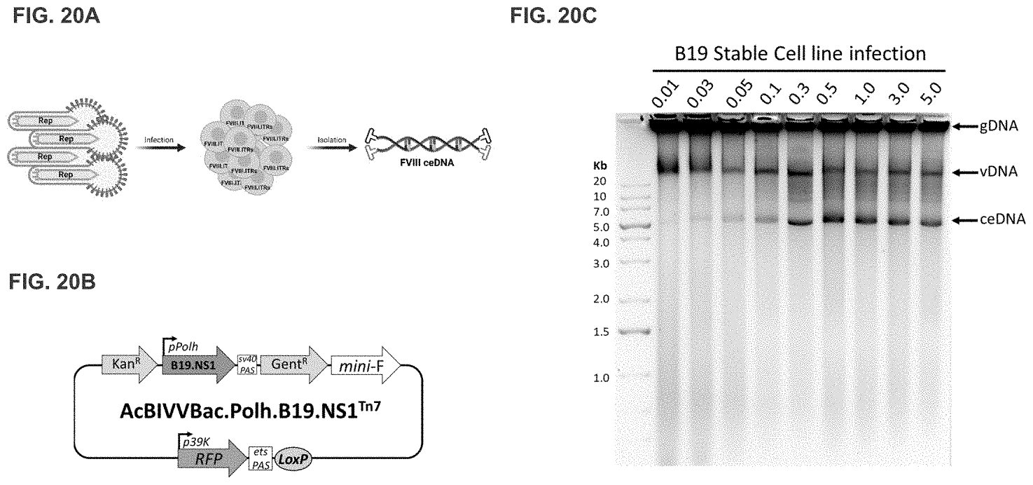

A cell line encoding V2.0 FVIIIXTEN with B19 Minimal ITRs was developed as described earlier (Example 8). Polyclonal Sf.FVIIIXTEN.B19.Minimal.ITRs cell population was then infected with the titrated working stock (P2) of AcBIVVBac.Polh.B19-NS1^{Tn7} BEV at an MOI of 0.01, 0.03, 0.05, 0.1, 0.3, 0.5, 1.0, 3.0, or 5.0 pfu/cell (FIG. 20A, 20B). Agarose gel analysis of ceFVIIIXTEN isolated from the B19 stable cell line showed increasing levels of DNA band intensity as the MOI of REP.BEV increases. However, the DNA band was slightly lower than the expected size of ceFVIIIXTEN (˜8.5 kb) (FIG. 20C). Interestingly, similar banding pattern was observed for the V1.0 ceFVIIIXTEN obtained from the B19 stable cell line (Example 9, FIG. 9A), though with different ITRs. In contrast, the ceFVIIIXTEN obtained from Sf9 cells infected with the B19 OneBAC or TwoBAC encoding FVIIIXTEN with B19 WT ITRs showed expected size DNA bands for V2.0 ceFVIIIXTEN (˜8.5 kb) (FIG. 15C & FIG. 17C).

These results suggest that though terminal resolution site and REP binding elements were present in the truncated or minimal variant of B19 ITRs, full-length (WT) sequence may be required for the B19-NS1 to bind and nick the DNA for efficient replication.

ceFVIIIXTEN GPV ITRs

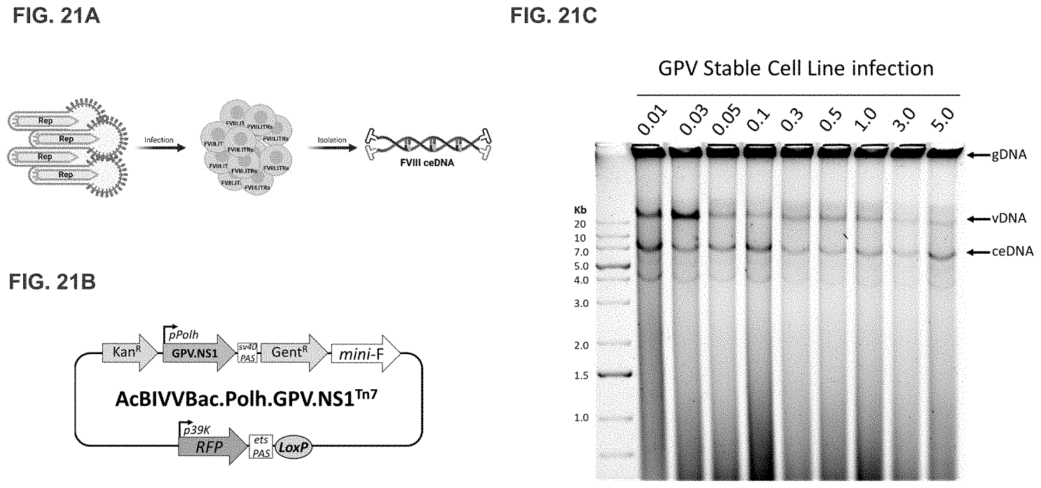

A cell line encoding V2.0 FVIIIXTEN with GPVΔ120 ITRs was developed as described earlier (Example 8). Polyclonal Sf.FVIIIXTEN.GPVΔ120.ITRs cell population was then infected with the titrated working stock (P2) of AcBIVVBac.Polh.GPV-NS1^{Tn7} BEV at an MOI of 0.01, 0.03, 0.05, 0.1, 0.3, 0.5, 1.0, 3.0, or 5.0 pfu/cell (FIG. 21A, 21B). Unlike B19 cell line, agarose gel analysis of ceFVIIIXTEN isolated from the GPV stable cell line showed reducing levels of productivity as the MOIs of infection increases.

Interestingly, for ceFVIIIXTEN GPV ITRs, the reverse trend was observed in ceFVIIIXTEN productivity in comparison with the other parvoviral ITRs. The lowest MOI (0.01 pfu/cell) used for infection in GPV cell line showed the highest band intensity for the ceFVIIIXTEN (ceDNA) in comparison with the other MOIs tested (FIG. 21C). These results suggest that very low level of GPV-NS1 is required for the GPV-ITR-mediated ceDNA production at least in the case of a stable cell line. Furthermore, this data suggests that the lower productivity of ceFVIIIXTEN observed in GPV OneBAC or TwoBAC (FIG. 7C, 18C) was probably due to the high virus load, which indirectly corresponds to the higher level of GPV-NS1 expression more than originally required for ceFVIIIXTEN production.

Nevertheless, the stable cell line approach does prove the concept of ceDNA production from the cell line stably integrated with the FVIIIXTEN expression cassette flanked with non-AAV parvoviral ITRs.

### Example 17: FVIIIXTEN ceDNA (ceFVIIIXTEN) Vector Purification

In the baculovirus-insect cell system, recombinant BEV delivers the gene of interest under a strong promoter and provides transcriptional complex essential for the virus replication in insect cells. Typically, the baculovirus DNA genome replicates in the nucleus and produce several tens of millions of progeny virus particles, each containing a full-length DNA genome. It has been demonstrated that baculoviral genomic DNAs are co-purified with the ceDNA while isolating DNA from the insect cells using a plasmid DNA-based purification method such as silica gel columns. The commercial plasmid DNA kit columns are generally not designed to separate DNA based on their molecular weights and therefore, typically, all forms of DNA present in the sample can bind to these columns. Moreover, the binding capacity of large molecular weight DNA could be different than the low molecular weight DNA and the anion-exchange based kit columns are not optimized based on the binding efficiency of different sizes of DNA.

It was hypothesized that the high molecular weight DNAs (>20 kb) observed in ceDNA preps were most likely baculoviral and/or Sf9 cell genomic DNAs that were co-purified with the low molecular weight ceFVIIIXTEN (˜8.5 kb) (see, e.g., FIGS. 14C-21C). Previously, an indirect approach was employed to reduce the baculoviral DNA by knocking out a baculoviral capsid gene such as VP80, which is required for the infectious progeny virus production. This approach showed significant reduction in the baculoviral DNA in the ceDNA prep obtained from the knock-out BEV (see U.S. Patent Application No. 63/069,115). Though this approach was efficient in reducing the baculoviral DNA contamination, it was unable to reduce the cellular genomic DNAs, which were present in a significant quantity (˜60%) of the total DNAs obtained from infected cell pellets.

In this study, a direct approach of separating FVIIIXTEN ceDNA from the rest of the unwanted DNAs was employed and demonstrated to efficiently obtain the purified FVIIIXTEN (>95% purity) from the total DNA prep from the infected cell pellet. This novel approach leverages preparative electrophoresis, which is widely used for separating different protein molecules according to their size and charge. See, e.g., Michov, B. (2020) Electrophoresis. Berlin, Boston: De Gruyter, pp. 405-424. For example, the Bio-Rad Model 491 prep cell or other such units can be used to separate complex molecules based on their sizes.

In the current study, a preparative electrophoresis technique was employed for separating FVIIIXTEN ceDNA from high molecular weight DNAs and successfully obtained the purified ceDNA to be used for in vivo studies, as described below.

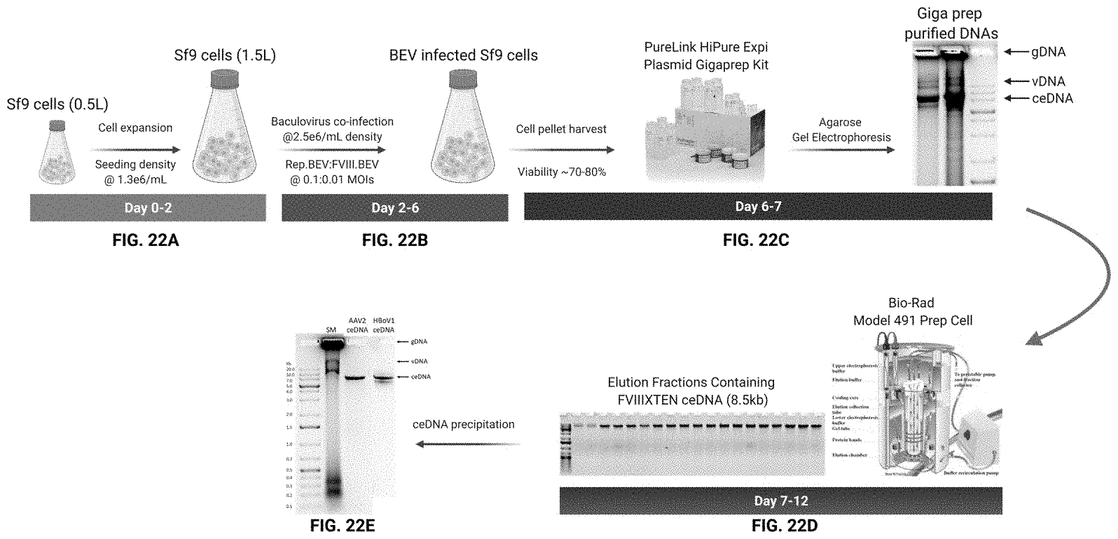

The entire workflow of ceDNA purification is shown in FIG. 22, where the process starts with scaling up the Sf9 cell culture from 0.5 L to 1.5 L or higher volume in serum-free insect cell culture medium (FIG. 22A). Upon reaching the desired cell density of ˜2.5×10⁶/mL, typically after 2 days of incubation with a seeding density of ˜1.3×10⁶/mL, cells are infected with OneBAC or TwoBAC BEVs (depending on the approach used for ceDNA production) at an optimized MOI and let the cells incubated in 28° C. shaker incubator until the viability reached at ˜60-70% which typically takes about 4 days (FIG. 22B). Once the viability reached at ˜70%, cells are harvested and processed for total DNA purification by anion-exchange chromatographic kit columns, such as PureLink HiPure Expi Plasmid Gigaprep purification kit (Invitrogen), according to the manufacturer's instructions. An aliquot of purified DNA material is checked on 0.8 to 1.2% agarose gel electrophoresis to determine the DNA productivity and integrity (FIG. 22C). The purified material is then loaded onto a Preparative Agarose Gel Electrophoresis Unit, containing a 0.5% preparative agarose gel and a 0.25% stacking agarose gel, assembled according to the manufacturer's instructions. Samples are run at low voltage (˜40 constant volts) at 4° C. for 6-7 days with a buffer recirculation flow rate of ˜50 mL/min and the elution buffer rate of 50 μL/min to collect each fraction at 70-80 min in the fraction collection chamber. After continuous elution electrophoresis, 20 μL of each fraction is checked on 0.8 to 1.2% agarose gel electrophoresis to determine the purity of FVIIIXTEN ceDNA (FIG. 22D). The desired fractions are combined to precipitate with 3M NaOAc, pH 5.5 and 100% EtOH at −20 C for 1-2 h. Finally, the precipitated FVIIIXTEN ceDNA is pelleted at high-speed and washed once with 70% EtOH before re-suspending into the TE, pH 8.0 buffer. Purified FVIIIXTEN ceDNA is again checked on 0.8 to 1.2% agarose gel electrophoresis to confirm the purity and integrity before injecting into the animals for in vivo efficacy studies (FIG. 22E).

### Example 18: FVIIIXTEN ceDNA (ceFVIIIXTEN) In Vivo Efficacy

ceFVIIIXTEN Systemic Administration in HemA Mice

To validate the functionality of ceDNA in vivo, purified ceFVIIIXTEN with AAV2 or HBoV1 WT ITRs was injected systemically via hydrodynamic tail-vein injections in hFVIIIR593C^{+/+}/HemA mice at 0.3 μg, 1.0 μg, or 2.0 μg/mouse, which is equivalent to 12 μg, 40 μg, and 80 μg/kg, respectively. Plasma samples from injected mice were collected at 7 days interval and FVIII activity was measured by the chromogenic assay, as described above.

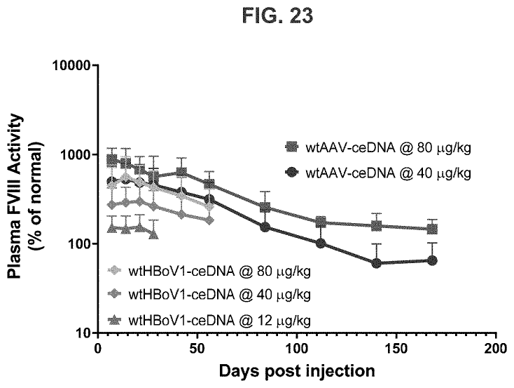

The plasma FVIII activity normalized to percent of normal for ceFVIIIXTEN injected cohorts is shown in FIG. 23. The results showed dose-dependent response in HemA mice with supraphysiological levels (>500% of normal) of FVIII expression observed in the highest dose of AAV2 or HBoV1 ceDNA tested. However, a gradual decline in FVIII expression levels was observed up to day 140 post injection, after which the levels were stabilized in ceFVIIIXTEN AAV2 ITRs injected cohorts. Interestingly, the FVIII expression levels of ceFVIIIXTEN HBoV1 ITRs showed a similar expression trend as the mice injected with ceFVIIIXTEN AAV2 ITRs (data not shown).

In conclusion, these in vivo efficacy studies validate the functionality of ceFVIIIXTEN and and demonstrates that parvoviral ITRs can be used to produce functional ceDNA encoding a transgene of interest in the baculovirus insect cell system.

### Example 19: FVIIIXTEN HBoV1 ITRs ceDNA Vector Production from OneBAC

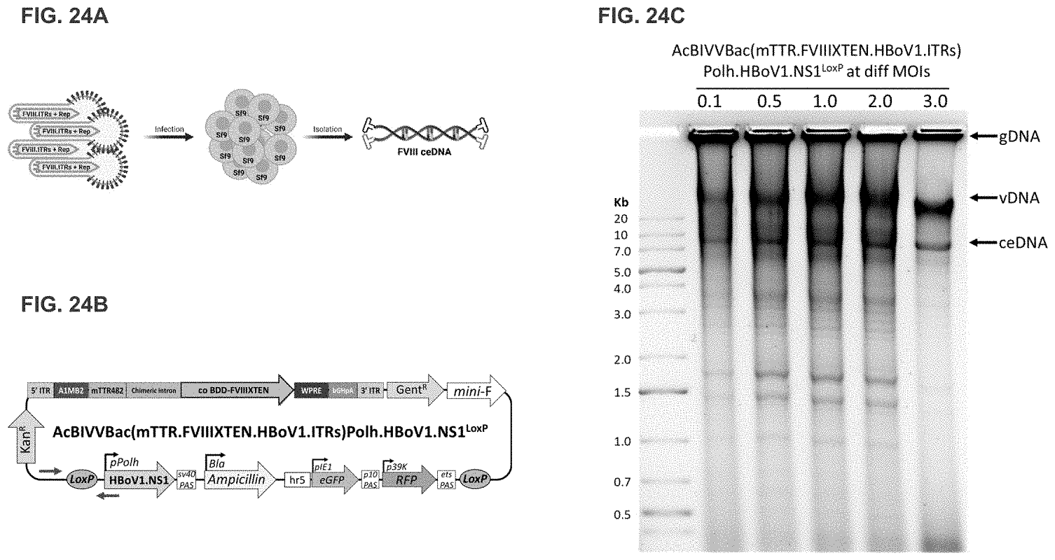

The OneBAC BEVs encoding both FVIIIXTEN HBoV1 ITRs and HBoV1 NS1 genes were tested for FVIIIXTEN ceDNA production in Sf9 cells. About 2.5×10⁶/mL cells were infected with the titrated working stock (P2) of each BEV at multiplicity of infections (MO's) of 0.1, 0.5, 1.0, 2.0, or 3.0 plaque forming units (pfu)/cell (FIG. 24A). Cells were suspended into 50 mL of serum-free ESF-921 medium and then incubated for 72-96 h or until the cell viability reached at 60-70% in 28° C. shaker incubator. At ˜96 h post-infection, infected cells were harvested, and the pellets were processed for FVIIIXTEN ceDNA vector isolation by PureLink Maxi Prep DNA isolation kit (Invitrogen), according to the manufacturer's instructions. Final elution fractions were analyzed on 0.8 to 1.2% agarose gel electrophoresis to determine the productivity of FVIIIXTEN ceDNA vector.

Agarose gel analysis of the AcBIVVBac (mTTR.FVIIIXTEN.HBoV1.ITRs)Polh.HBoV1.NS1^{LoxP} BEV (FIG. 24B) encoding FVIIIXTEN with HBoV1 ITRs and polyhedrin-driven HBoV1-NS1 is shown in FIG. 24C. The results showed DNA band corresponds to the size of FVIIIXTEN HBoV1 ITRs (˜8.5 kb) ceDNA in all the doses tested with increasing productivity as the MOI increases.

This result was contrarary to the ceDNA productivity obtained with AAV2 ITRs OneBAC, where reduction in productivity was previously observed with increases in viral load. Without being bound by theory, the HBoV1-NS1 protein may have a unique mechanism of binding and endonuclease activity at the terminal resolution site of HBoV1 ITRs for DNA replication which could be due to the distinct structures of REH and LEH ITRs.

In conclusion, these experiments have shown that the OneBAC approach does prove the concept of ceDNA production from a single recombinant BEV encoding FVIIIXTEN with HBoV1 ITRs and NS1 transgenes. It also shows the feasibility and functionality of multiple transgenes inserted at different loci in a baculovirus shuttle vector (BIVVBac), and its potential use for recombinant AAV vector production in the baculovirus insect cell system.

### Example 20: FVIIIXTEN HBoV1 ITRs ceDNA Vector Production from TwoBAC

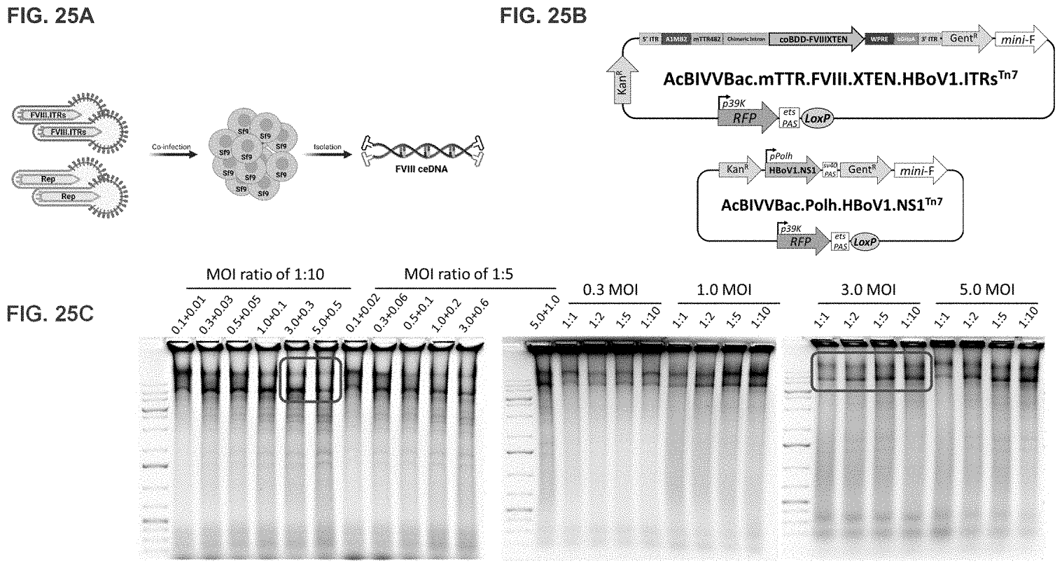

To investigate the TwoBAC approach to transgene expression, clonal recombinant BEV encoding FVIIIXTEN HBoV1 ITRs with polyhedrin-driven HBoV1-NS1 BEV were tested for co-infections at different MOIs of 1:10 and 1:5 ratios or at different ratios of an MOI of 0.3, 1.0, 3.0, and 5.0 pfu/cell for FVIIIXTEN ceDNA vector production in Sf9 cells (FIG. 25A). Specifically, —2.0×10⁶/mL cells were seeded in 50 mL of serum-free ESF-921 medium and co-infected with titrated working stocks (P2) of AcBIVVBac.mTTR.FVIIIXTEN.HBoV1.ITRs^{Tn7} BEV at an MOI of 0.1, 0.3, 0.5, 1.0, 3.0, 5.0 pfu/cell with AcBIVVBac.Polh.HBoV1-NS1^{Tn7} BEV at an MOI of 0.01, 0.03, 0.05, 0.1, 0.3, 0.5 pfu/cell for keeping the constant 1:10 ratio or at an MOI of 0.02, 0.06, 0.1, 0.2, 0.6, 1.0 pfu/cell for keeping the constant 1:5 ratio, respectively. Similarly, cells were also co-infected at 1:1, 1:2, 1:5, or 1:10 ratio at a constant MOI of 0.3, 1.0, 3.0, or 5.0 pfu/cell (FIG. 25B). In each case, virus inoculum was not removed, and the cells were incubated until the viability reached at 60-70% in 28° C. shaker incubator. At ˜96 h post-infection, infected cells were harvested, and the pellets were processed for FVIIIXTEN ceDNA vector isolation by PureLink Maxi Prep DNA isolation kit (Invitrogen), according to the manufacturer's instructions. Final elution fractions were analyzed on 0.8 to 1.2% agarose gel electrophoresis to determine the ceDNA productivity.

As expected, agarose gel analyses showed varying degree of FVIIIXTEN ceDNA productivity with different conditions. However, TwoBAC co-infected at an MOI of 3.0 pfu/cell showed increasing levels of FVIIIXTEN ceDNA productivity with increases in ratios of virus load with 1:10 being the highest in comparison to the other conditions tested (FIG. 25C). The higher virus load appears to improve the productivity of FVIIIXTEN HBoV1 ITRs ceDNA, which is consistent with the observation in the OneBAC BEVs (see Example 6). This further suggests the requirement of higher level of HBoV1-NS1 for HBoV1-ITR-dependent FVIIIXTEN ceDNA replication in Sf9 cells.

The results with OneBAC or TwoBAC indicate that the level of HBoV1-NS1 replication has a significant impact on the FVIIIXTEN ceDNA productivity in the baculovirus system.

In conclusion, these experiments have shown that the TwoBAC approach does prove the concept of ceDNA production from two recombinant BEVs encoding FVIIIXTEN with HBoV1 ITRs and/or NS1 transgene. These experiments also demonstrate the significance of optimum MOI ratio and/or promoter for achieving higher productivity of FVIIIXTEN ceDNA in Sf9 cells.

### Sequences

TABLE 6
Additional nucleotide or amino acid sequences.

| SEQ ID NO | |
|---|---|
| and | |
| Description | Nucleotide or Amino Acid Sequence |
| SEQ ID NO: | CCAAATCAGATGCCGCCGGTCGCCGCCGGTAGGCGGGACTTCCGGTACAGGCGCGCCGCTGTACCGGAAGTCCCG |
| 16 | CCTACCGGCGGCGACCGGCGGCATCTGATTTGGTGTCTTCTTTTAAATTTT |
| B19 Minimal | |
| ITR | |
| SEQ ID NO. | CTAGCGAGCTCATTGGAGGGTTCGTTCGTTCGAACCAGCCAATCAGGGGAGGGGGAAGTGACGCAAGTTCCGGTC |
| 17 | ACATGCTTCCGGTGACGCACATCCGGTGACGTAGTTCCGGTCACGTGCTTCCTGTCACGTGTTTCCGGTCGCATG |
| GPVΔ120 | CTCACGTGACCGGAAACACGTGACAGGAAGCACGTGACCGGAACTACGTCACCGGATGTGCGTCACCGGAAGCAT |
| ITR: | GTGACCGGAACTTGCGTCACTTCCCCCTCCCCTGATTGGCTGGTTCGAACGAACGAACCCTCCAATGAGACTCAA |
| | GGACAAGAGGATATTTTGCGCGCCAGGAAGTGT |
| SEQ ID NO. | CTAGCGAGCTCATTGGAGGGTTCGTTCGTTCGAACCAGCCAATCAGGGGAGGGGGAAGTGACGCAAGTTCCGGTC |
| 18 | ACATGCTTCCGGTGACGCACATCCGGTGACGTAGTTCGCATGCGAACTACGTCACCGGATGTGCGTCACCGGAAG |
| GPVΔ186 | CATGTGACCGGAACTTGCGTCACTTCCCCCTCCCCTGATTGGCTGGTTCGAACGAACGAACCCTCCAATGAGACT |
| ITR: | CAAGGACAAGAGGATATTTTGCGCGCCAGGAAGTGT |
| SEQ ID NO: | GGCCCCAGGTTAATTTTTAAAAAGCAGTCAAAGGTCAAAGTGGCCCTTGGCAGCATTTACTCTCTCTATTGACTTTGGT |
| 19 | TAATAATCTCAGGAGCACAAACATTCCTGGAGGCAGGAGAAGAAATCAACATCCTGGACTTATCCTCTGGGCCTCTCCC |
| V2.0 | CACCTTCGATGGCCCCAGGTTAATTTTTAAAAAGCAGTCAAAGGTCAAAGTGGCCCTTGGCAGCATTTACTCTCTCTAT |
| Expression | TGACTTTGGTTAATAATCTCAGGAGCACAAACATTCCTGGAGGCAGGAGAAGAAATCAACATCCTGGACTTATCCTCTG |
| cassette | GGCCTCTCCCCACCGATATCTACCTGCTGATCGCCCGGCCCCTGTTCAAACATGTCCTAATACTCTGTCGGGGCAAAGG |
| mTTR482- | TCGGCAGTAGTTTTCCATCTTACTCAACATCCTCCCAGTGTACGTAGGATCCTGTCTGTCTGCACATTTCGTAGAGCGA |
| Intron- | GTGTTCCGATACTCTAATCTCCCGGGGCAAAGGTCGTATTGACTTAGGTTACTTATTCTCCTTTTGTTGACTAAGTCAA |
| coBDDFVIIIX | TAATCAGAATCAGCAGGTTTGGAGTCAGCTTGGCAGGGATCAGCAGCCTGGGTTGGAAGGAGGGGGTATAAAAGCCCCT |
| TEN (V2.0)- | TCACCAGGAGAAGCCGTCACACAGATCCACAAGCTCCTGCTAGGAATTCTCAGGAGCACAAACATTCCTGGAGGCAGGA |
| WPRE- | GAAGAAATCAACATCCTGGACTTATCCTCTGGGCCTCTCCCCACCGATATCTACCTGCTGATCGCCCGGCCCCTGTTCA |
| bGHPolyA | AACATGTCCTAATACTCTGTCGGGGCAAAGGTCGGCAGTAGTTTTCCATCTTACTCAACATCCTCCCAGTGTACGTAGG |
| | ATCCTGTCTGTCTGCACATTTCGTAGAGCGAGTGTTCCGATACTCTAATCTCCCGGGGCAAAGGTCGTATTGACTTAGG |
| | TTACTTATTCTCCTTTTGTTGACTAAGTCAATAATCAGAATCAGCAGGTTTGGAGTCAGCTTGGCAGGGATCAGCAGCC |
| | TGGGTTGGAAGGAGGGGGTATAAAAGCCCCTTCACCAGGAGAAGCCGTCACACAGATCCACAAGCTCCTGCTAGAGTCG |
| | CTGCGCGCTGCCTTCGCCCCGTGCCCCGCTCCGCCGCCGCCTCGCGCCGCCCGCCCCGGCTCTGACTGACCGCGTTACT |
| | CCCACAGGTGAGCGGGCGGGACGGCCCTTCTCCTCCGGGCTGTAATTAGCGCTTGGTTTATTGACGGCTTGTTTCTTTT |
| | CTGTGGCTGCGTGAAAGCCTTGAGGGGCTCCGGGAAGGCCCTTTGTGCGGGGGGAGCGGCTCGGGGGGTGCGTGCGTGT |
| | GTGTGTGCGTGGGGAGCGCCGCGTGCGGCTCCGCGCTGCCCGGCGGCTGTGAGCGCTGCGGGCGCGGCGCGGGGCTTTG |
| | TGCGCTCCGCAGTGTGCGCGAGGGGAGCGCGGCCGGGGGCGGTGCCCCGCGGTGCGGGGGGGGCTGCGAGGGGAACAAA |
| | GGCTGCGTGCGGGGTGTGTGCGTGGGGGGGTGAGCAGGGGGTGTGGGCGCGTCGGTCGGGCTGCAACCCCCCCTGCACC |
| | CCCCTCCCCGAGTTGCTGAGCACGGCCCGGCTTCGGGTGCGGGGCTCCGTACGGGGCGTGGCGCGGGGCTCGCCGTGCC |
| | GGGCGGGGGGTGGCGGCAGGTGGGGGTGCCGGGCGGGGCGGGGCCGCCTCGGGCCGGGGAGGGCTCGGGGGAGGGGCGC |
| | GGCGGCCCCCGGAGCGCCGGCGGCTGTCGAGGCGCGGCGAGCCGCAGCCATTGCCTTTTATGGTAATCGTGCGAGAGGG |
| | CGCAGGGACTTCCTTTGTCCCAAATCTGTGCGGAGCCGAAATCTGGGAGGCGCCGCCGCACCCCCTCTAGCGGGCGCGG |
| | GGCGAAGCGGTGCGGCGCCGGCAGGAAGGAAATGGGCGGGGAGGGCCTTCGTGCGTCGCCGCGCCGCCGTCCCCTTCTC |
| | CCTCTCCAGCCTCGGGGCTGTCCGCGGGGGGACGGCTGCCTTCGGGGGGGACGGGGCAGGGCGGGGTTCGGCTTCTGGC |
| | GTGTGACCGGCGGCTCTAGAGCCTCTGCTAACCTTGTTCTTGCCTTCTTCTTTTTCCTACAGCTCCTGGGCAACGTGCT |
| | GGTTATTGTGCTGTCTCATCATTTTGGCAAAGAATTACTCGAGGCCACCATGCAGATTGAACTGTCCACTTGCTTCTTC |
| | CTGTGCCTCCTGCGGTTTTGCTTCTCGGCCACCCGCCGGTATTACTTAGGTGCTGTGGAACTGAGCTGGGACTACATGC |
| | AGTCCGACCTGGGAGAACTGCCGGTGGACGCGAGATTCCCACCTAGAGTCCCGAAGTCCTTCCCATTCAACACCTCCGT |
| | GGTCTACAAAAAGACCCTGTTCGTGGAGTTCACTGACCACCTTTTCAATATTGCCAAGCCGCGCCCCCCCTGGATGGGC |
| | CTGCTTGGTCCTACGATCCAAGCAGAGGTCTACGACACCGTGGTCATCACACTGAAGAACATGGCCTCACACCCCGTGT |
| | CGCTGCATGCTGTGGGAGTGTCCTACTGGAAGGCCTCAGAGGGTGCCGAATATGATGACCAGACCAGCCAGAGGGAAAA |
| | GGAGGATGACAAAGTGTTCCCGGGTGGCAGCCACACTTACGTGTGGCAAGTGCTGAAGGAAAACGGGCCTATGGCGTCG |
| | GACCCCCTATGCCTGACCTACTCCTACCTGTCCCATGTGGACCTTGTGAAGGATCTCAACTCGGGACTGATCGGCGCCC |
| | TCTTGGTGTGCAGAGAAGGCAGCCTGGCGAAGGAAAAGACTCAGACCCTGCACAAGTTCATTCTGTTGTTTGCTGTGTT |
| | CGATGAAGGAAAGTCCTGGCACTCAGAAACCAAGAACTCGCTGATGCAGGATAGAGATGCGGCCTCGGCCAGAGCCTGG |
| | CCTAAAATGCACACCGTCAACGGATATGTGAACAGGTCGCTCCCTGGCCTCATCGGCTGCCACAGAAAGTCCGTGTATT |
| | GGCATGTGATCGGCATGGGTACTACTCCGGAAGTGCATAGTATCTTTCTGGAGGGCCATACCTTCTTGGTGCGCAACCA |
| | CAGACAGGCCTCGCTGGAAATCTCGCCTATCACTTTCTTGACTGCGCAGACCCTCCTTATGGACCTTGGACAGTTCCTG |
| | CTGTTCTGTCACATCAGCTCCCATCAGCATGATGGGATGGAGGCCTATGTCAAAGTGGACTCCTGCCCTGAGGAGCCAC |
| | AGCTCCGGATGAAGAACAATGAGGAAGCGGAGGATTACGACGACGACCTGACTGACAGCGAAATGGACGTCGTGCGATT |
| | CGATGACGACAACAGCCCGTCCTTCATCCAAATTAGATCAGTGGCGAAGAAGCACCCCAAGACCTGGGTGCACTACATT |
| | GCCGCCGAGGAAGAGGACTGGGACTACGCGCCGCTGGTGCTGGCGCCAGACGACAGGAGCTACAAGTCCCAGTACCTCA |
| | ACAACGGGCCGCAGCGCATTGGCAGGAAGTACAAGAAAGTCCGCTTCATGGCCTACACTGATGAAACCTTCAAGACGAG |
| | GGAAGCCATCCAGCACGAGTCAGGCATCCTGGGACCGCTCCTTTACGGCGAAGTCGGGGATACCCTGCTCATCATTTTC |
| | AAGAACCAGGCATCGCGGCCCTACAACATCTACCCTCACGGGATCACAGACGTGCGCCCGCTCTACTCCCGCCGGCTGC |
| | CCAAGGGAGTGAAGCACCTGAAGGATTTTCCCATCCTGCCGGGAGAAATCTTCAAGTACAAGTGGACCGTGACTGTGGA |
| | AGATGGCCCTACCAAGTCGGACCCTCGCTGTCTGACCCGGTACTATTCCTCGTTTGTGAACATGGAGCGCGACCTGGCC |
| | TCGGGGCTGATTGGTCCGCTGCTGATCTGCTACAAGGAGTCCGTGGACCAGCGCGGGAACCAGATCATGTCCGACAAGC |
| | GCAACGTGATCCTGTTCTCTGTCTTTGATGAAAACAGATCGTGGTACTTGACTGAGAATATCCAGCGGTTCCTGCCCAA |
| | CCCAGCGGGAGTGCAACTGGAGGACCCGGAGTTCCAGGCCTCAAACATTATGCACTCTATCAACGGCTATGTGTTCGAC |
| | TCGCTCCAACTGAGCGTGTGCCTGCATGAAGTGGCATACTGGTACATTCTGTCCATCGGAGCCCAGACCGACTTCCTGT |
| | CCGTGTTCTTCTCCGGATACACCTTCAAGCATAAGATGGTGTACGAGGACACTCTGACCCTCTTCCCATTTTCCGGAGA |
| | AACTGTGTTCATGTCAATGGAAAACCCGGGCTTGTGGATTCTGGGTTGCCATAACTCGGACTTCCGGAATAGAGGGATG |
| | ACCGCCCTGCTGAAAGTGTCCAGCTGTGACAAGAATACCGGCGATTACTACGAGGACAGCTATGAGGACATCTCCGCTT |
| | ATCTGCTGTCCAAGAACAACGCCATTGAACCCAGGTCCTTCTCCCAAAACGGTGCACCGACCTCCGAAAGCGCCACCCC |
| | AGAGTCAGGACCTGGCTCGGAACCGGCTACCTCGGGCTCAGAGACACCGGGGACTTCCGAGTCCGCAACCCCCGAGAGT |
| | GGACCCGGATCCGAACCAGCAACCTCAGGATCAGAAACCCCGGGAACTTCGGAATCCGCCACTCCCGAGTCGGGACCAG |
| | GCACCTCCACTGAGCCTTCCGAGGGAAGCGCCCCCGGATCCCCTGCTGGATCCCCTACCAGCACTGAAGAAGGCACCTC |
| | AGAATCCGCGACCCCTGAGTCCGGCCCTGGAAGCGAACCCGCCACCTCCGGTTCCGAAACCCCTGGGACTAGCGAGAGC |
| | GCCACTCCGGAATCGGGCCCAGGAAGCCCTGCCGGATCCCCGACCAGCACCGAGGAGGGAAGCCCCGCCGGGTCACCGA |
| | CTTCCACTGAGGAGGGAGCCTCATCCCCCCCCGTGCTGAAGCGGCATCAAAGAGAGATCACCAGGACCACTCTCCAGTC |
| | CGATCAGGAAGAAATTGACTACGACGATACTATCAGCGTGGAGATGAAGAAGGAGGACTTCGACATCTACGATGAGGAT |
| | GAGAACCAGTCCCCTCGGAGCTTTCAGAAGAAAACCCGCCACTACTTCATCGCTGCCGTGGAGCGGCTGTGGGATTACG |
| | GGATGTCCAGCTCACCGCATGTGCTGCGGAATAGAGCGCAGTCAGGATCGGTGCCCCAGTTCAAGAAGGTCGTGTTCCA |
| | AGAGTTCACCGACGGGTCCTTCACTCAACCCCTGTACCGGGGCGAACTCAACGAACACCTGGGACTGCTTGGGCCGTAT |
| | ATCAGGGCAGAAGTGGAAGATAACATCATGGTCACCTTCCGCAACCAGGCCTCCCGGCCGTACAGCTTCTACTCTTCAC |
| | TGATCTCCTACGAGGAAGATCAGCGGCAGGGAGCCGAGCCCCGGAAGAACTTCGTCAAGCCTAACGAAACTAAGACCTA |
| | CTTTTGGAAGGTCCAGCATCACATGGCCCCGACCAAAGACGAGTTCGACTGTAAAGCCTGGGCCTACTTCTCCGATGTG |
| | GACCTGGAGAAGGACGTGCACTCGGGACTCATTGGCCCGCTCCTTGTGTGCCATACTAATACCCTGAACCCTGCTCACG |
| | GTCGCCAAGTCACAGTGCAGGAGTTCGCCCTCTTCTTCACCATCTTCGATGAAACAAAGTCCTGGTACTTTACTGAGAA |
| | CATGGAACGCAATTGCAGGGCACCCTGCAACATCCAGATGGAAGATCCCACCTTCAAGGAAAACTACCGGTTTCATGCC |
| | ATTAACGGCTACATAATGGACACGTTGCCAGGACTGGTCATGGCCCAGGACCAGAGAATCCGGTGGTATCTGCTCTCCA |
| | TGGGCTCCAACGAAAACATTCACAGCATTCATTTTTCCGGCCATGTGTTCACCGTCCGGAAGAAGGAAGAGTACAAGAT |
| | GGCTCTGTACAACCTCTACCCTGGAGTGTTCGAGACTGTGGAAATGCTGCCTAGCAAGGCCGGCATTTGGAGAGTGGAA |
| | TGCCTGATCGGAGAGCATTTGCACGCCGGAATGTCCACCCTGTTTCTTGTGTACTCCAACAAGTGCCAGACCCCGCTGG |
| | GAATGGCCTCAGGTCATATTAGGGATTTCCAGATCACTGCTTCGGGGCAGTACGGGCAGTGGGCACCTAAGTTGGCCCG |
| | GCTGCACTACTCTGGCTCCATCAATGCCTGGTCCACCAAGGAACCCTTCTCCTGGATTAAGGTGGACCTCCTGGCCCCA |
| | ATGATTATTCACGGTATTAAGACCCAGGGTGCCCGACAGAAGTTCTCCTCACTCTACATCTCGCAATTCATCATAATGT |
| | ACAGCCTGGATGGGAAGAAGTGGCAGACCTACCGGGGAAACTCCACTGGAACGCTCATGGTGTTTTTCGGCAACGTGGA |
| | CTCCTCCGGCATTAAGCACAACATCTTCAACCCTCCGATCATTGCTCGGTACATCCGGCTGCACCCAACTCACTACAGC |
| | ATCCGGTCCACCCTGCGGATGGAACTGATGGGTTGTGACCTGAACTCCTGCTCCATGCCCCTTGGGATGGAATCCAAGG |
| | CCATTAGCGATGCACAGATCACCGCCTCTTCATACTTCACCAACATGTTCGCGACCTGGTCCCCGTCGAAGGCCCGCCT |
| | GCACCTCCAAGGTCGCTCCAATGCGTGGCGGCCTCAAGTGAACAACCCCAAGGAGTGGCTCCAGGTCGACTTCCAAAAG |
| | ACCATGAAGGTCACCGGAGTGACCACCCAGGGCGTGAAGTCCCTGCTGACCTCTATGTACGTTAAGGAGTTCCTCATCT |
| | CCTCAAGCCAAGACGGACATCAGTGGACCCTGTTCTTCCAAAACGGAAAAGTCAAAGTATTCCAGGGCAACCAGGACTC |
| | CTTCACCCCTGTGGTCAACAGCCTGGACCCCCCATTGCTGACCCGCTACCTCCGCATCCACCCCCAAAGCTGGGTCCAC |
| | CAGATCGCACTGCGCATGGAGGTCCTTGGATGCGAAGCCCAAGATCTGTACTAAGCGGCCGCTCATAATCAACCTCTGG |
| | ATTACAAAATTTGTGAAAGATTGACTGGTATTCTTAACTATGTTGCTCCTTTTACGCTATGTGGATACGCTGCTTTAAT |
| | GCCTTTGTATCATGCTATTGCTTCCCGTATGGCTTTCATTTTCTCCTCCTTGTATAAATCCTGGTTGCTGTCTCTTTAT |
| | GAGGAGTTGTGGCCCGTTGTCAGGCAACGTGGCGTGGTGTGCACTGTGTTTGCTGACGCAACCCCCACTGGTTGGGGCA |
| | TTGCCACCACCTGTCAGCTCCTTTCCGGGACTTTCGCTTTCCCCCTCCCTATTGCCACGGCGGAACTCATCGCCGCCTG |
| | CCTTGCCCGCTGCTGGACAGGGGCTCGGCTGTTGGGCACTGACAATTCCGTGGTGTTGTCGGGGAAATCATCGTCCTTT |
| | CCTTGGCTGCTCGCCTGTGTTGCCACCTGGATTCTGCGCGGGACGTCCTTCTGCTACGTCCCTTCGGCCCTCAATCCAG |
| | CGGACCTTCCTTCCCGCGGCCTGCTGCCGGCTCTGCGGCCTCTTCCGCGTCTTCGCCTTCGCCCTCAGACGAGTCGGAT |
| | CTCCCTTTGGGCCGCCTCCCCGCTGCCTAGGCGACTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAAAATGAGGAAATTGCATCGCATTGTCTGAG |
| | TAGGTGTCATTCTATTCTGGGGGGTGGGGTGGGGCAGGACAGCAAGGGGGAGGATTGGGAAGACAATAGCAGGCATGCT |
| | GGGGAAGACCATGGGCGCGCCAGGCCTGTCGACGCCCGGGCGGTACCGCGATCGCTCGCGACGCATAAAG |
| SEQ ID NO: | ATGCAGATTGAACTGTCCACTTGCTTCTTCCTGTGCCTCCTGCGGTTTTGCTTCTCGGCCACCCGCCGGTATTACTTAG |
| 20 | GTGCTGTGGAACTGAGCTGGGACTACATGCAGTCCGACCTGGGAGAACTGCCGGTGGACGCGAGATTCCCACCTAGAGT |
| Nucleotide | CCCGAAGTCCTTCCCATTCAACACCTCCGTGGTCTACAAAAAGACCCTGTTCGTGGAGTTCACTGACCACCTTTTCAAT |
| sequence | ATTGCCAAGCCGCGCCCCCCCTGGATGGGCCTGCTTGGTCCTACGATCCAAGCAGAGGTCTACGACACCGTGGTCATCA |
| encoding | CACTGAAGAACATGGCCTCACACCCCGTGTCGCTGCATGCTGTGGGAGTGTCCTACTGGAAGGCCTCAGAGGGTGCCGA |
| coBDDFVIIIX | ATATGATGACCAGACCAGCCAGAGGGAAAAGGAGGATGACAAAGTGTTCCCGGGTGGCAGCCACACTTACGTGTGGCAA |
| TEN (V2.0) | GTGCTGAAGGAAAACGGGCCTATGGCGTCGGACCCCCTATGCCTGACCTACTCCTACCTGTCCCATGTGGACCTTGTGA |
| | AGGATCTCAACTCGGGACTGATCGGCGCCCTCTTGGTGTGCAGAGAAGGCAGCCTGGCGAAGGAAAAGACTCAGACCCT |
| | GCACAAGTTCATTCTGTTGTTTGCTGTGTTCGATGAAGGAAAGTCCTGGCACTCAGAAACCAAGAACTCGCTGATGCAG |
| | GATAGAGATGCGGCCTCGGCCAGAGCCTGGCCTAAAATGCACACCGTCAACGGATATGTGAACAGGTCGCTCCCTGGCC |
| | TCATCGGCTGCCACAGAAAGTCCGTGTATTGGCATGTGATCGGCATGGGTACTACTCCGGAAGTGCATAGTATCTTTCT |
| | GGAGGGCCATACCTTCTTGGTGCGCAACCACAGACAGGCCTCGCTGGAAATCTCGCCTATCACTTTCTTGACTGCGCAG |
| | ACCCTCCTTATGGACCTTGGACAGTTCCTGCTGTTCTGTCACATCAGCTCCCATCAGCATGATGGGATGGAGGCCTATG |
| | TCAAAGTGGACTCCTGCCCTGAGGAGCCACAGCTCCGGATGAAGAACAATGAGGAAGCGGAGGATTACGACGACGACCT |
| | GACTGACAGCGAAATGGACGTCGTGCGATTCGATGACGACAACAGCCCGTCCTTCATCCAAATTAGATCAGTGGCGAAG |
| | AAGCACCCCAAGACCTGGGTGCACTACATTGCCGCCGAGGAAGAGGACTGGGACTACGCGCCGCTGGTGCTGGCGCCAG |
| | ACGACAGGAGCTACAAGTCCCAGTACCTCAACAACGGGCCGCAGCGCATTGGCAGGAAGTACAAGAAAGTCCGCTTCAT |
| | GGCCTACACTGATGAAACCTTCAAGACGAGGGAAGCCATCCAGCACGAGTCAGGCATCCTGGGACCGCTCCTTTACGGC |
| | GAAGTCGGGGATACCCTGCTCATCATTTTCAAGAACCAGGCATCGCGGCCCTACAACATCTACCCTCACGGGATCACAG |
| | ACGTGCGCCCGCTCTACTCCCGCCGGCTGCCCAAGGGAGTGAAGCACCTGAAGGATTTTCCCATCCTGCCGGGAGAAAT |
| | CTTCAAGTACAAGTGGACCGTGACTGTGGAAGATGGCCCTACCAAGTCGGACCCTCGCTGTCTGACCCGGTACTATTCC |
| | TCGTTTGTGAACATGGAGCGCGACCTGGCCTCGGGGCTGATTGGTCCGCTGCTGATCTGCTACAAGGAGTCCGTGGACC |
| | AGCGCGGGAACCAGATCATGTCCGACAAGCGCAACGTGATCCTGTTCTCTGTCTTTGATGAAAACAGATCGTGGTACTT |
| | GACTGAGAATATCCAGCGGTTCCTGCCCAACCCAGCGGGAGTGCAACTGGAGGACCCGGAGTTCCAGGCCTCAAACATT |
| | ATGCACTCTATCAACGGCTATGTGTTCGACTCGCTCCAACTGAGCGTGTGCCTGCATGAAGTGGCATACTGGTACATTC |
| | TGTCCATCGGAGCCCAGACCGACTTCCTGTCCGTGTTCTTCTCCGGATACACCTTCAAGCATAAGATGGTGTACGAGGA |
| | CACTCTGACCCTCTTCCCATTTTCCGGAGAAACTGTGTTCATGTCAATGGAAAACCCGGGCTTGTGGATTCTGGGTTGC |
| | CATAACTCGGACTTCCGGAATAGAGGGATGACCGCCCTGCTGAAAGTGTCCAGCTGTGACAAGAATACCGGCGATTACT |
| | ACGAGGACAGCTATGAGGACATCTCCGCTTATCTGCTGTCCAAGAACAACGCCATTGAACCCAGGTCCTTCTCCCAAAA |
| | CGGTGCACCGACCTCCGAAAGCGCCACCCCAGAGTCAGGACCTGGCTCGGAACCGGCTACCTCGGGCTCAGAGACACCG |
| | GGGACTTCCGAGTCCGCAACCCCCGAGAGTGGACCCGGATCCGAACCAGCAACCTCAGGATCAGAAACCCCGGGAACTT |
| | CGGAATCCGCCACTCCCGAGTCGGGACCAGGCACCTCCACTGAGCCTTCCGAGGGAAGCGCCCCCGGATCCCCTGCTGG |
| | ATCCCCTACCAGCACTGAAGAAGGCACCTCAGAATCCGCGACCCCTGAGTCCGGCCCTGGAAGCGAACCCGCCACCTCC |
| | GGTTCCGAAACCCCTGGGACTAGCGAGAGCGCCACTCCGGAATCGGGCCCAGGAAGCCCTGCCGGATCCCCGACCAGCA |
| | CCGAGGAGGGAAGCCCCGCCGGGTCACCGACTTCCACTGAGGAGGGAGCCTCATCCCCCCCCGTGCTGAAGCGGCATCA |
| | AAGAGAGATCACCAGGACCACTCTCCAGTCCGATCAGGAAGAAATTGACTACGACGATACTATCAGCGTGGAGATGAAG |
| | AAGGAGGACTTCGACATCTACGATGAGGATGAGAACCAGTCCCCTCGGAGCTTTCAGAAGAAAACCCGCCACTACTTCA |
| | TCGCTGCCGTGGAGCGGCTGTGGGATTACGGGATGTCCAGCTCACCGCATGTGCTGCGGAATAGAGCGCAGTCAGGATC |
| | GGTGCCCCAGTTCAAGAAGGTCGTGTTCCAAGAGTTCACCGACGGGTCCTTCACTCAACCCCTGTACCGGGGCGAACTC |
| | AACGAACACCTGGGACTGCTTGGGCCGTATATCAGGGCAGAAGTGGAAGATAACATCATGGTCACCTTCCGCAACCAGG |
| | CCTCCCGGCCGTACAGCTTCTACTCTTCACTGATCTCCTACGAGGAAGATCAGCGGCAGGGAGCCGAGCCCCGGAAGAA |
| | CTTCGTCAAGCCTAACGAAACTAAGACCTACTTTTGGAAGGTCCAGCATCACATGGCCCCGACCAAAGACGAGTTCGAC |
| | TGTAAAGCCTGGGCCTACTTCTCCGATGTGGACCTGGAGAAGGACGTGCACTCGGGACTCATTGGCCCGCTCCTTGTGT |
| | GCCATACTAATACCCTGAACCCTGCTCACGGTCGCCAAGTCACAGTGCAGGAGTTCGCCCTCTTCTTCACCATCTTCGA |
| | TGAAACAAAGTCCTGGTACTTTACTGAGAACATGGAACGCAATTGCAGGGCACCCTGCAACATCCAGATGGAAGATCCC |
| | ACCTTCAAGGAAAACTACCGGTTTCATGCCATTAACGGCTACATAATGGACACGTTGCCAGGACTGGTCATGGCCCAGG |
| | ACCAGAGAATCCGGTGGTATCTGCTCTCCATGGGCTCCAACGAAAACATTCACAGCATTCATTTTTCCGGCCATGTGTT |
| | CACCGTCCGGAAGAAGGAAGAGTACAAGATGGCTCTGTACAACCTCTACCCTGGAGTGTTCGAGACTGTGGAAATGCTG |
| | CCTAGCAAGGCCGGCATTTGGAGAGTGGAATGCCTGATCGGAGAGCATTTGCACGCCGGAATGTCCACCCTGTTTCTTG |
| | TGTACTCCAACAAGTGCCAGACCCCGCTGGGAATGGCCTCAGGTCATATTAGGGATTTCCAGATCACTGCTTCGGGGCA |
| | GTACGGGCAGTGGGCACCTAAGTTGGCCCGGCTGCACTACTCTGGCTCCATCAATGCCTGGTCCACCAAGGAACCCTTC |
| | TCCTGGATTAAGGTGGACCTCCTGGCCCCAATGATTATTCACGGTATTAAGACCCAGGGTGCCCGACAGAAGTTCTCCT |
| | CACTCTACATCTCGCAATTCATCATAATGTACAGCCTGGATGGGAAGAAGTGGCAGACCTACCGGGGAAACTCCACTGG |
| | AACGCTCATGGTGTTTTTCGGCAACGTGGACTCCTCCGGCATTAAGCACAACATCTTCAACCCTCCGATCATTGCTCGG |
| | TACATCCGGCTGCACCCAACTCACTACAGCATCCGGTCCACCCTGCGGATGGAACTGATGGGTTGTGACCTGAACTCCT |
| | GCTCCATGCCCCTTGGGATGGAATCCAAGGCCATTAGCGATGCACAGATCACCGCCTCTTCATACTTCACCAACATGTT |
| | CGCGACCTGGTCCCCGTCGAAGGCCCGCCTGCACCTCCAAGGTCGCTCCAATGCGTGGCGGCCTCAAGTGAACAACCCC |
| | AAGGAGTGGCTCCAGGTCGACTTCCAAAAGACCATGAAGGTCACCGGAGTGACCACCCAGGGCGTGAAGTCCCTGCTGA |
| | CCTCTATGTACGTTAAGGAGTTCCTCATCTCCTCAAGCCAAGACGGACATCAGTGGACCCTGTTCTTCCAAAACGGAAA |
| | AGTCAAAGTATTCCAGGGCAACCAGGACTCCTTCACCCCTGTGGTCAACAGCCTGGACCCCCCATTGCTGACCCGCTAC |
| | CTCCGCATCCACCCCCAAAGCTGGGTCCACCAGATCGCACTGCGCATGGAGGTCCTTGGATGCGAAGCCCAAGATCTGT |
| | ACTAA |
| SEQ ID NO: | GGCCCCAGGTTAATTTTTAAAAAGCAGTCAAAGGTCAAAGTGGCCCTTGGCAGCATTTACTCTCTCTATTGACTTTGGT |
| 21 | taataatctcaggagcacaaacattcctggaggcaggagaagaaatcaacatcctggacttatcctctgggcctctccc |
| A1MB2 | CACCTTCGATGGCCCCAGGTTAATTTTTAAAAAGCAGTCAAAGGTCAAAGTGGCCCTTGGCAGCATTTACTCTCTCTAT |
| enhancer | TGACTTTGGTTAATAATCTCAGGAGCACAAACATTCCTGGAGGCAGGAGAAGAAATCAACATCCTGGACTTATCCTCTG |
| | GGCCTCTCCCCACC |
| SEQ ID NO: | GATATCTACCTGCTGATCGCCCGGCCCCTGTTCAAACATGTCCTAATACTCTGTCGGGGCAAAGGTCGGCAGTAGTTTT |
| 22 | CCATCTTACTCAACATCCTCCCAGTGTACGTAGGATCCTGTCTGTCTGCACATTTCGTAGAGCGAGTGTTCCGATACTC |
| mTTR | TAATCTCCCGGGGCAAAGGTCGTATTGACTTAGGTTACTTATTCTCCTTTTGTTGACTAAGTCAATAATCAGAATCAGC |
| promoter | AGGTTTGGAGTCAGCTTGGCAGGGATCAGCAGCCTGGGTTGGAAGGAGGGGGTATAAAAGCCCCTTCACCAGGAGAAGC |
| | CGTCACACAGATCCACAAGCTCCTGCTAG |
| SEQ ID NO: | TCAGGAGCACAAACATTCCTGGAGGCAGGAGAAGAAATCAACATCCTGGACTTATCCTCTGGGCCTCTCCCCACC |
| 23 | GATATCTACCTGCTGATCGCCCGGCCCCTGTTCAAACATGTCCTAATACTCTGTCGGGGCAAAGGTCGGCAGTAG |
| Chimeric | TTTTCCATCTTACTCAACATCCTCCCAGTGTACGTAGGATCCTGTCTGTCTGCACATTTCGTAGAGCGAGTGTTC |
| Intron | CGATACTCTAATCTCCCGGGGCAAAGGTCGTATTGACTTAGGTTACTTATTCTCCTTTTGTTGACTAAGTCAATA |
| | ATCAGAATCAGCAGGTTTGGAGTCAGCTTGGCAGGGATCAGCAGCCTGGGTTGGAAGGAGGGGGTATAAAAGCCC |
| | CTTCACCAGGAGAAGCCGTCACACAGATCCACAAGCTCCTGCTAGAGTCGCTGCGCGCTGCCTTCGCCCCGTGCC |
| | CCGCTCCGCCGCCGCCTCGCGCCGCCCGCCCCGGCTCTGACTGACCGCGTTACTCCCACAGGTGAGCGGGCGGGA |
| | CGGCCCTTCTCCTCCGGGCTGTAATTAGCGCTTGGTTTATTGACGGCTTGTTTCTTTTCTGTGGCTGCGTGAAAG |
| | CCTTGAGGGGCTCCGGGAAGGCCCTTTGTGCGGGGGGAGCGGCTCGGGGGGTGCGTGCGTGTGTGTGTGCGTGGG |
| | GAGCGCCGCGTGCGGCTCCGCGCTGCCCGGCGGCTGTGAGCGCTGCGGGCGCGGCGCGGGGCTTTGTGCGCTCCG |
| | CAGTGTGCGCGAGGGGAGCGCGGCCGGGGGCGGTGCCCCGCGGTGCGGGGGGGGCTGCGAGGGGAACAAAGGCTG |
| | CGTGCGGGGTGTGTGCGTGGGGGGGTGAGCAGGGGGTGTGGGCGCGTCGGTCGGGCTGCAACCCCCCCTGCACCC |
| | CCCTCCCCGAGTTGCTGAGCACGGCCCGGCTTCGGGTGCGGGGCTCCGTACGGGGCGTGGCGCGGGGCTCGCCGT |
| | GCCGGGCGGGGGGTGGCGGCAGGTGGGGGTGCCGGGCGGGGCGGGGCCGCCTCGGGCCGGGGAGGGCTCGGGGGA |
| | GGGGCGCGGCGGCCCCCGGAGCGCCGGCGGCTGTCGAGGCGCGGCGAGCCGCAGCCATTGCCTTTTATGGTAATC |
| | GTGCGAGAGGGCGCAGGGACTTCCTTTGTCCCAAATCTGTGCGGAGCCGAAATCTGGGAGGCGCCGCCGCACCCC |
| | CTCTAGCGGGCGCGGGGCGAAGCGGTGCGGCGCCGGCAGGAAGGAAATGGGCGGGGAGGGCCTTCGTGCGTCGCC |
| | GCGCCGCCGTCCCCTTCTCCCTCTCCAGCCTCGGGGCTGTCCGCGGGGGGACGGCTGCCTTCGGGGGGGACGGGG |
| | CAGGGCGGGGTTCGGCTTCTGGCGTGTGACCGGCGGCTCTAGAGCCTCTGCTAACCTTGTTCTTGCCTTCTTCTT |
| | TTTCCTACAGCTCCTGGGCAACGTGCTGGTTATTGTGCTGTCTCATCATTTTGGCAAAGAATTA |
| SEQ ID NO: | TCATAATCAACCTCTGGATTACAAAATTTGTGAAAGATTGACTGGTATTCTTAACTATGTTGCTCCTTTTACGCTATGT |
| 24 | GGATACGCTGCTTTAATGCCTTTGTATCATGCTATTGCTTCCCGTATGGCTTTCATTTTCTCCTCCTTGTATAAATCCT |
| WPRE | GGTTGCTGTCTCTTTATGAGGAGTTGTGGCCCGTTGTCAGGCAACGTGGCGTGGTGTGCACTGTGTTTGCTGACGCAAC |
| | CCCCACTGGTTGGGGCATTGCCACCACCTGTCAGCTCCTTTCCGGGACTTTCGCTTTCCCCCTCCCTATTGCCACGGCG |
| | GAACTCATCGCCGCCTGCCTTGCCCGCTGCTGGACAGGGGCTCGGCTGTTGGGCACTGACAATTCCGTGGTGTTGTCGG |
| | GGAAATCATCGTCCTTTCCTTGGCTGCTCGCCTGTGTTGCCACCTGGATTCTGCGCGGGACGTCCTTCTGCTACGTCCC |
| | TTCGGCCCTCAATCCAGCGGACCTTCCTTCCCGCGGCCTGCTGCCGGCTCTGCGGCCTCTTCCGCGTCTTCGCCTTCGC |
| | CCTCAGACGAGTCGGATCTCCCTTTGGGCCGCCTCCCCGCTG |
| SEQ ID NO: | CGACTGTGCCTTCTAGTTGCCAGCCATCTGTTGTTTGCCCCTCCCCCGTGCCTTCCTTGACCCTGGAAGGTGCCA |
| 25 | CTCCCACTGTCCTTTCCTAATAAAATGAGGAAATTGCATCGCATTGTCTGAGTAGGTGTCATTCTATTCTGGGGG |
| bGHpA | GTGGGGTGGGGCAGGACAGCAAGGGGGAGGATTGGGAAGACAATAGCAGGCATGCTGGGGA |
| SEQ ID NO: | GCCACTCGCCGGTACTACCTTGGAGCCGTGGAGCTTTCATGGGACTACATGCAGAGCGACCTGGGCGAACTCCCC |
| 28 | GTGGATGCCAGATTCCCCCCCCGCGTGCCAAAGTCCTTCCCCTTTAACACCTCCGTGGTGTACAAGAAAACCCTC |
| Nucleotide | TTTGTCGAGTTCACTGACCACCTGTTCAACATCGCCAAGCCGCGCCCACCTTGGATGGGCCTCCTGGGACCGACC |
| sequence | ATTCAAGCTGAAGTGTACGACACCGTGGTGATCACCCTGAAGAACATGGCGTCCCACCCCGTGTCCCTGCATGCG |
| encoding | GTCGGAGTGTCCTACTGGAAGGCCTCCGAAGGAGCTGAGTACGACGACCAGACTAGCCAGCGGGAAAAGGAGGAC |
| BDD-co6FVIII | GATAAAGTGTTCCCGGGCGGCTCGCATACTTACGTGTGGCAAGTCCTGAAGGAAAACGGACCTATGGCATCCGAT |
| (V1.0) | CCTCTGTGCCTGACTTACTCCTACCTTTCCCATGTGGACCTCGTGAAGGACCTGAACAGCGGGCTGATTGGTGCA |
| (no XTEN) | CTTCTCGTGTGCCGCGAAGGTTCGCTCGCTAAGGAAAAGACCCAGACCCTCCATAAGTTCATCCTTTTGTTCGCT |
| | GTGTTCGATGAAGGAAAGTCATGGCATTCCGAAACTAAGAACTCGCTGATGCAGGACCGGGATGCCGCCTCAGCC |
| | CGCGCCTGGCCTAAAATGCATACAGTCAACGGATACGTGAATCGGTCACTGCCCGGGCTCATCGGTTGTCACAGA |
| | AAGTCCGTGTACTGGCACGTCATCGGCATGGGCACTACGCCTGAAGTGCACTCCATCTTCCTGGAAGGGCACACC |
| | TTCCTCGTGCGCAACCACCGCCAGGCCTCTCTGGAAATCTCCCCGATTACCTTTCTGACCGCCCAGACTCTGCTC |
| | ATGGACCTGGGGCAGTTCCTTCTCTTCTGCCACATCTCCAGCCATCAGCACGACGGAATGGAGGCCTACGTGAAG |
| | GTGGACTCATGCCCGGAAGAACCTCAGTTGCGGATGAAGAACAACGAGGAGGCCGAGGACTATGACGACGATTTG |
| | ACTGACTCCGAGATGGACGTCGTGCGGTTCGATGACGACAACAGCCCCAGCTTCATCCAGATTCGCAGCGTGGCC |
| | AAGAAGCACCCCAAAACCTGGGTGCACTACATCGCGGCCGAGGAAGAAGATTGGGACTACGCCCCGTTGGTGCTG |
| | GCACCCGATGACCGGTCGTACAAGTCCCAGTATCTGAACAATGGTCCGCAGCGGATTGGCAGAAAGTACAAGAAA |
| | GTGCGGTTCATGGCGTACACTGACGAAACGTTTAAGACCCGGGAGGCCATTCAACATGAGAGCGGCATTCTGGGA |
| | CCACTGCTGTACGGAGAGGTCGGCGATACCCTGCTCATCATCTTCAAAAACCAGGCCTCCCGGCCTTACAACATC |
| | TACCCTCACGGAATCACCGACGTGCGGCCACTCTACTCGCGGCGCCTGCCGAAGGGCGTCAAGCACCTGAAAGAC |
| | TTCCCTATCCTGCCGGGCGAAATCTTCAAGTATAAGTGGACCGTCACCGTGGAGGACGGGCCCACCAAGAGCGAT |
| | CCTAGGTGTCTGACTCGGTACTACTCCAGCTTCGTGAACATGGAACGGGACCTGGCATCGGGACTCATTGGACCG |
| | CTGCTGATCTGCTACAAAGAGTCGGTGGATCAACGCGGCAACCAGATCATGTCCGACAAGCGCAACGTGATCCTG |
| | TTCTCCGTGTTTGATGAAAACAGATCCTGGTACCTCACTGAAAACATCCAGAGGTTCCTCCCAAACCCCGCAGGA |
| | GTGCAACTGGAGGACCCTGAGTTTCAGGCCTCGAATATCATGCACTCGATTAACGGTTACGTGTTCGACTCGCTG |
| | CAGCTGAGCGTGTGCCTCCATGAAGTCGCTTACTGGTACATTCTGTCCATCGGCGCCCAGACTGACTTCCTGAGC |
| | GTGTTCTTTTCCGGTTACACCTTTAAGCACAAGATGGTGTACGAAGATACCCTGACCCTGTTCCCTTTCTCCGGC |
| | GAAACGGTGTTCATGTCGATGGAGAACCCGGGTCTGTGGATTCTGGGATGCCACAACAGCGACTTTCGGAACCGC |
| | GGAATGACTGCCCTGCTGAAGGTGTCCTCATGCGACAAGAACACCGGAGACTACTACGAGGACTCCTACGAGGAT |
| | ATCTCAGCCTACCTCCTGTCCAAGAACAACGCGATCGAGCCGCGCAGCTTCAGCCAGAACCCGCCTGTGCTGAAG |
| | AGGCACCAGCGAGAAATTACCCGGACCACCCTCCAATCGGATCAGGAGGAAATCGACTACGACGACACCATCTCG |
| | GTGGAAATGAAGAAGGAAGATTTCGATATCTACGACGAGGACGAAAATCAGTCCCCTCGCTCATTCCAAAAGAAA |
| | ACTAGACACTACTTTATCGCCGCGGTGGAAAGACTGTGGGACTATGGAATGTCATCCAGCCCTCACGTCCTTCGG |
| | AACCGGGCCCAGAGCGGATCGGTGCCTCAGTTCAAGAAAGTGGTGTTCCAGGAGTTCACCGACGGCAGCTTCACC |
| | CAGCCGCTGTACCGGGGAGAACTGAACGAACACCTGGGCCTGCTCGGTCCCTACATCCGCGCGGAAGTGGAGGAT |
| | AACATCATGGTGACCTTCCGTAACCAAGCATCCAGACCTTACTCCTTCTATTCCTCCCTGATCTCATACGAGGAG |
| | GACCAGCGCCAAGGCGCCGAGCCCCGCAAGAACTTCGTCAAGCCCAACGAGACTAAGACCTACTTCTGGAAGGTC |
| | CAACACCATATGGCCCCGACCAAGGATGAGTTTGACTGCAAGGCCTGGGCCTACTTCTCCGACGTGGACCTTGAG |
| | AAGGATGTCCATTCCGGCCTGATCGGGCCGCTGCTCGTGTGTCACACCAACACCCTGAACCCAGCGCATGGACGC |
| | CAGGTCACCGTCCAGGAGTTTGCTCTGTTCTTCACCATTTTTGACGAAACTAAGTCCTGGTACTTCACCGAGAAT |
| | ATGGAGCGAAACTGTAGAGCGCCCTGCAATATCCAGATGGAAGATCCGACTTTCAAGGAGAACTATAGATTCCAC |
| | GCCATCAACGGGTACATCATGGATACTCTGCCGGGGCTGGTCATGGCCCAGGATCAGAGGATTCGGTGGTACTTG |
| | CTGTCAATGGGATCGAACGAAAACATTCACTCCATTCACTTCTCCGGTCACGTGTTCACTGTGCGCAAGAAGGAG |
| | GAGTACAAGATGGCGCTGTACAATCTGTACCCCGGGGTGTTCGAAACTGTGGAGATGCTGCCGTCCAAGGCCGGC |
| | ATCTGGAGAGTGGAGTGCCTGATCGGAGAGCACCTCCACGCGGGGATGTCCACCCTCTTCCTGGTGTACTCGAAT |
| | AAGTGCCAGACCCCGCTGGGCATGGCCTCGGGCCACATCAGAGACTTCCAGATCACAGCAAGCGGACAATACGGC |
| | CAATGGGCGCCGAAGCTGGCCCGCTTGCACTACTCCGGATCGATCAACGCATGGTCCACCAAGGAACCGTTCTCG |
| | TGGATTAAGGTGGACCTCCTGGCCCCTATGATTATCCACGGAATTAAGACCCAGGGCGCCAGGCAGAAGTTCTCC |
| | TCCCTGTACATCTCGCAATTCATCATCATGTACAGCCTGGACGGGAAGAAGTGGCAGACTTACAGGGGAAACTCC |
| | ACCGGCACCCTGATGGTCTTTTTCGGCAACGTGGATTCCTCCGGCATTAAGCACAACATCTTCAACCCACCGATC |
| | ATAGCCAGATATATTAGGCTCCACCCCACTCACTACTCAATCCGCTCAACTCTTCGGATGGAACTCATGGGGTGC |
| | GACCTGAACTCCTGCTCCATGCCGTTGGGGATGGAATCAAAGGCTATTAGCGACGCCCAGATCACCGCGAGCTCC |
| | TACTTCACTAACATGTTCGCCACCTGGAGCCCCTCCAAGGCCAGGCTGCACTTGCAGGGACGGTCAAATGCCTGG |
| | CGGCCGCAAGTGAACAATCCGAAGGAATGGCTTCAAGTGGATTTCCAAAAGACCATGAAAGTGACCGGAGTCACC |
| | ACCCAGGGAGTGAAGTCCCTTCTGACCTCGATGTATGTGAAGGAGTTCCTGATTAGCAGCAGCCAGGACGGGCAC |
| | CAGTGGACCCTGTTCTTCCAAAACGGAAAGGTCAAGGTGTTCCAGGGGAACCAGGACTCGTTCACACCCGTGGTG |
| | AACTCCCTGGACCCCCCACTGCTGACGCGGTACTTGAGGATTCATCCTCAGTCCTGGGTCCATCAGATTGCATTG |
| | CGAATGGAAGTCCTGGGCTGCGAGGCCCAGGACCTGTACTGA |
| SEQ ID NO: | GCCACCCGCCGGTATTACTTAGGTGCTGTGGAACTGAGCTGGGACTACATGCAGTCCGACCTGGGAGAACTGCCG |
| 29 | GTGGACGCGAGATTCCCACCTAGAGTCCCGAAGTCCTTCCCATTCAACACCTCCGTGGTCTACAAAAAGACCCTG |
| Nucleotide | TTCGTGGAGTTCACTGACCACCTTTTCAATATTGCCAAGCCGCGCCCCCCCTGGATGGGCCTGCTTGGTCCTACG |
| sequence | ATCCAAGCAGAGGTCTACGACACCGTGGTCATCACACTGAAGAACATGGCCTCACACCCCGTGTCGCTGCATGCT |
| encoding | GTGGGAGTGTCCTACTGGAAGGCCTCAGAGGGTGCCGAATATGATGACCAGACCAGCCAGAGGGAAAAGGAGGAT |
| coBDDFVIII | GACAAAGTGTTCCCGGGTGGCAGCCACACTTACGTGTGGCAAGTGCTGAAGGAAAACGGGCCTATGGCGTCGGAC |
| (V2.0) | CCCCTATGCCTGACCTACTCCTACCTGTCCCATGTGGACCTTGTGAAGGATCTCAACTCGGGACTGATCGGCGCC |
| (no XTEN) | CTCTTGGTGTGCAGAGAAGGCAGCCTGGCGAAGGAAAAGACTCAGACCCTGCACAAGTTCATTCTGTTGTTTGCT |
| | GTGTTCGATGAAGGAAAGTCCTGGCACTCAGAAACCAAGAACTCGCTGATGCAGGATAGAGATGCGGCCTCGGCC |
| | AGAGCCTGGCCTAAAATGCACACCGTCAACGGATATGTGAACAGGTCGCTCCCTGGCCTCATCGGCTGCCACAGA |
| | AAGTCCGTGTATTGGCATGTGATCGGCATGGGTACTACTCCGGAAGTGCATAGTATCTTTCTGGAGGGCCATACC |
| | TTCTTGGTGCGCAACCACAGACAGGCCTCGCTGGAAATCTCGCCTATCACTTTCTTGACTGCGCAGACCCTCCTT |
| | ATGGACCTTGGACAGTTCCTGCTGTTCTGTCACATCAGCTCCCATCAGCATGATGGGATGGAGGCCTATGTCAAA |
| | GTGGACTCCTGCCCTGAGGAGCCACAGCTCCGGATGAAGAACAATGAGGAAGCGGAGGATTACGACGACGACCTG |
| | ACTGACAGCGAAATGGACGTCGTGCGATTCGATGACGACAACAGCCCGTCCTTCATCCAAATTAGATCAGTGGCG |
| | AAGAAGCACCCCAAGACCTGGGTGCACTACATTGCCGCCGAGGAAGAGGACTGGGACTACGCGCCGCTGGTGCTG |
| | GCGCCAGACGACAGGAGCTACAAGTCCCAGTACCTCAACAACGGGCCGCAGCGCATTGGCAGGAAGTACAAGAAA |
| | GTCCGCTTCATGGCCTACACTGATGAAACCTTCAAGACGAGGGAAGCCATCCAGCACGAGTCAGGCATCCTGGGA |
| | CCGCTCCTTTACGGCGAAGTCGGGGATACCCTGCTCATCATTTTCAAGAACCAGGCATCGCGGCCCTACAACATC |
| | TACCCTCACGGGATCACAGACGTGCGCCCGCTCTACTCCCGCCGGCTGCCCAAGGGAGTGAAGCACCTGAAGGAT |
| | TTTCCCATCCTGCCGGGAGAAATCTTCAAGTACAAGTGGACCGTGACTGTGGAAGATGGCCCTACCAAGTCGGAC |
| | CCTCGCTGTCTGACCCGGTACTATTCCTCGTTTGTGAACATGGAGCGCGACCTGGCCTCGGGGCTGATTGGTCCG |
| | CTGCTGATCTGCTACAAGGAGTCCGTGGACCAGCGCGGGAACCAGATCATGTCCGACAAGCGCAACGTGATCCTG |
| | TTCTCTGTCTTTGATGAAAACAGATCGTGGTACTTGACTGAGAATATCCAGCGGTTCCTGCCCAACCCAGCGGGA |
| | GTGCAACTGGAGGACCCGGAGTTCCAGGCCTCAAACATTATGCACTCTATCAACGGCTATGTGTTCGACTCGCTC |
| | CAACTGAGCGTGTGCCTGCATGAAGTGGCATACTGGTACATTCTGTCCATCGGAGCCCAGACCGACTTCCTGTCC |
| | GTGTTCTTCTCCGGATACACCTTCAAGCATAAGATGGTGTACGAGGACACTCTGACCCTCTTCCCATTTTCCGGA |
| | GAAACTGTGTTCATGTCAATGGAAAACCCGGGCTTGTGGATTCTGGGTTGCCATAACTCGGACTTCCGGAATAGA |
| | GGGATGACCGCCCTGCTGAAAGTGTCCAGCTGTGACAAGAATACCGGCGATTACTACGAGGACAGCTATGAGGAC |
| | ATCTCCGCTTATCTGCTGTCCAAGAACAACGCCATTGAACCCAGGTCCTTCTCCCAAAACGGTGCACCGGCCTCA |
| | TCCCCCCCCGTGCTGAAGCGGCATCAAAGAGAGATCACCAGGACCACTCTCCAGTCCGATCAGGAAGAAATTGAC |
| | TACGACGATACTATCAGCGTGGAGATGAAGAAGGAGGACTTCGACATCTACGATGAGGATGAGAACCAGTCCCCT |
| | CGGAGCTTTCAGAAGAAAACCCGCCACTACTTCATCGCTGCCGTGGAGCGGCTGTGGGATTACGGGATGTCCAGC |
| | TCACCGCATGTGCTGCGGAATAGAGCGCAGTCAGGATCGGTGCCCCAGTTCAAGAAGGTCGTGTTCCAAGAGTTC |
| | ACCGACGGGTCCTTCACTCAACCCCTGTACCGGGGCGAACTCAACGAACACCTGGGACTGCTTGGGCCGTATATC |
| | AGGGCAGAAGTGGAAGATAACATCATGGTCACCTTCCGCAACCAGGCCTCCCGGCCGTACAGCTTCTACTCTTCA |
| | CTGATCTCCTACGAGGAAGATCAGCGGCAGGGAGCCGAGCCCCGGAAGAACTTCGTCAAGCCTAACGAAACTAAG |
| | ACCTACTTTTGGAAGGTCCAGCATCACATGGCCCCGACCAAAGACGAGTTCGACTGTAAAGCCTGGGCCTACTTC |
| | TCCGATGTGGACCTGGAGAAGGACGTGCACTCGGGACTCATTGGCCCGCTCCTTGTGTGCCATACTAATACCCTG |
| | AACCCTGCTCACGGTCGCCAAGTCACAGTGCAGGAGTTCGCCCTCTTCTTCACCATCTTCGATGAAACAAAGTCC |
| | TGGTACTTTACTGAGAACATGGAACGCAATTGCAGGGCACCCTGCAACATCCAGATGGAAGATCCCACCTTCAAG |
| | GAAAACTACCGGTTTCATGCCATTAACGGCTACATAATGGACACGTTGCCAGGACTGGTCATGGCCCAGGACCAG |
| | AGAATCCGGTGGTATCTGCTCTCCATGGGCTCCAACGAAAACATTCACAGCATTCATTTTTCCGGCCATGTGTTC |
| | ACCGTCCGGAAGAAGGAAGAGTACAAGATGGCTCTGTACAACCTCTACCCTGGAGTGTTCGAGACTGTGGAAATG |
| | CTGCCTAGCAAGGCCGGCATTTGGAGAGTGGAATGCCTGATCGGAGAGCATTTGCACGCCGGAATGTCCACCCTG |
| | TTTCTTGTGTACTCCAACAAGTGCCAGACCCCGCTGGGAATGGCCTCAGGTCATATTAGGGATTTCCAGATCACT |
| | GCTTCGGGGCAGTACGGGCAGTGGGCACCTAAGTTGGCCCGGCTGCACTACTCTGGCTCCATCAATGCCTGGTCC |
| | ACCAAGGAACCCTTCTCCTGGATTAAGGTGGACCTCCTGGCCCCAATGATTATTCACGGTATTAAGACCCAGGGT |
| | GCCCGACAGAAGTTCTCCTCACTCTACATCTCGCAATTCATCATAATGTACAGCCTGGATGGGAAGAAGTGGCAG |
| | ACCTACCGGGGAAACTCCACTGGAACGCTCATGGTGTTTTTCGGCAACGTGGACTCCTCCGGCATTAAGCACAAC |
| | ATCTTCAACCCTCCGATCATTGCTCGGTACATCCGGCTGCACCCAACTCACTACAGCATCCGGTCCACCCTGCGG |
| | ATGGAACTGATGGGTTGTGACCTGAACTCCTGCTCCATGCCCCTTGGGATGGAATCCAAGGCCATTAGCGATGCA |
| | CAGATCACCGCCTCTTCATACTTCACCAACATGTTCGCGACCTGGTCCCCGTCGAAGGCCCGCCTGCACCTCCAA |
| | GGTCGCTCCAATGCGTGGCGGCCTCAAGTGAACAACCCCAAGGAGTGGCTCCAGGTCGACTTCCAAAAGACCATG |
| | AAGGTCACCGGAGTGACCACCCAGGGCGTGAAGTCCCTGCTGACCTCTATGTACGTTAAGGAGTTCCTCATCTCC |
| | TCAAGCCAAGACGGACATCAGTGGACCCTGTTCTTCCAAAACGGAAAAGTCAAAGTATTCCAGGGCAACCAGGAC |
| | TCCTTCACCCCTGTGGTCAACAGCCTGGACCCCCCATTGCTGACCCGCTACCTCCGCATCCACCCCCAAAGCTGG |
| | GTCCACCAGATCGCACTGCGCATGGAGGTCCTTGGATGCGAAGCCCAAGATCTGTACTAA |
| SEQ ID NO: | ATGCAGATTGAGCTGTCCACTTGTTTCTTCCTGTGCCTCCTGCGCTTCTGTTTCTCCGCCACTCGCCGGTACTAC |
| 30 | CTTGGAGCCGTGGAGCTTTCATGGGACTACATGCAGAGCGACCTGGGCGAACTCCCCGTGGATGCCAGATTCCCC |
| V1.0 | CCCCGCGTGCCAAAGTCCTTCCCCTTTAACACCTCCGTGGTGTACAAGAAAACCCTCTTTGTCGAGTTCACTGAC |
| Expression | CACCTGTTCAACATCGCCAAGCCGCGCCCACCTTGGATGGGCCTCCTGGGACCGACCATTCAAGCTGAAGTGTAC |
| cassette | GACACCGTGGTGATCACCCTGAAGAACATGGCGTCCCACCCCGTGTCCCTGCATGCGGTCGGAGTGTCCTACTGG |
| TTP-Intron- | AAGGCCTCCGAAGGAGCTGAGTACGACGACCAGACTAGCCAGCGGGAAAAGGAGGACGATAAAGTGTTCCCGGGC |
| BDDFVIIIco6 | GGCTCGCATACTTACGTGTGGCAAGTCCTGAAGGAAAACGGACCTATGGCATCCGATCCTCTGTGCCTGACTTAC |
| XTEN (V1.0)- | TCCTACCTTTCCCATGTGGACCTCGTGAAGGACCTGAACAGCGGGCTGATTGGTGCACTTCTCGTGTGCCGCGAA |
| WPRE- | GGTTCGCTCGCTAAGGAAAAGACCCAGACCCTCCATAAGTTCATCCTTTTGTTCGCTGTGTTCGATGAAGGAAAG |
| bGHPolyA | TCATGGCATTCCGAAACTAAGAACTCGCTGATGCAGGACCGGGATGCCGCCTCAGCCCGCGCCTGGCCTAAAATG |
| expression | CATACAGTCAACGGATACGTGAATCGGTCACTGCCCGGGCTCATCGGTTGTCACAGAAAGTCCGTGTACTGGCAC |
| cassette | GTCATCGGCATGGGCACTACGCCTGAAGTGCACTCCATCTTCCTGGAAGGGCACACCTTCCTCGTGCGCAACCAC |
| | CGCCAGGCCTCTCTGGAAATCTCCCCGATTACCTTTCTGACCGCCCAGACTCTGCTCATGGACCTGGGGCAGTTC |
| | CTTCTCTTCTGCCACATCTCCAGCCATCAGCACGACGGAATGGAGGCCTACGTGAAGGTGGACTCATGCCCGGAA |
| | GAACCTCAGTTGCGGATGAAGAACAACGAGGAGGCCGAGGACTATGACGACGATTTGACTGACTCCGAGATGGAC |
| | GTCGTGCGGTTCGATGACGACAACAGCCCCAGCTTCATCCAGATTCGCAGCGTGGCCAAGAAGCACCCCAAAACC |
| | TGGGTGCACTACATCGCGGCCGAGGAAGAAGATTGGGACTACGCCCCGTTGGTGCTGGCACCCGATGACCGGTCG |
| | TACAAGTCCCAGTATCTGAACAATGGTCCGCAGCGGATTGGCAGAAAGTACAAGAAAGTGCGGTTCATGGCGTAC |
| | ACTGACGAAACGTTTAAGACCCGGGAGGCCATTCAACATGAGAGCGGCATTCTGGGACCACTGCTGTACGGAGAG |
| | GTCGGCGATACCCTGCTCATCATCTTCAAAAACCAGGCCTCCCGGCCTTACAACATCTACCCTCACGGAATCACC |
| | GACGTGCGGCCACTCTACTCGCGGCGCCTGCCGAAGGGCGTCAAGCACCTGAAAGACTTCCCTATCCTGCCGGGC |
| | GAAATCTTCAAGTATAAGTGGACCGTCACCGTGGAGGACGGGCCCACCAAGAGCGATCCTAGGTGTCTGACTCGG |
| | TACTACTCCAGCTTCGTGAACATGGAACGGGACCTGGCATCGGGACTCATTGGACCGCTGCTGATCTGCTACAAA |
| | GAGTCGGTGGATCAACGCGGCAACCAGATCATGTCCGACAAGCGCAACGTGATCCTGTTCTCCGTGTTTGATGAA |
| | AACAGATCCTGGTACCTCACTGAAAACATCCAGAGGTTCCTCCCAAACCCCGCAGGAGTGCAACTGGAGGACCCT |
| | GAGTTTCAGGCCTCGAATATCATGCACTCGATTAACGGTTACGTGTTCGACTCGCTGCAACTGAGCGTGTGCCTC |
| | CATGAAGTCGCTTACTGGTACATTCTGTCCATCGGCGCCCAGACTGACTTCCTGAGCGTGTTCTTTTCCGGTTAC |
| | ACCTTTAAGCACAAGATGGTGTACGAAGATACCCTGACCCTGTTCCCTTTCTCCGGCGAAACGGTGTTCATGTCG |
| | ATGGAGAACCCGGGTCTGTGGATTCTGGGATGCCACAACAGCGACTTTCGGAACCGCGGAATGACTGCCCTGCTG |
| | AAGGTGTCCTCATGCGACAAGAACACCGGAGACTACTACGAGGACTCCTACGAGGATATCTCAGCCTACCTCCTG |
| | TCCAAGAACAACGCGATCGAGCCGCGCAGCTTCAGCCAGAACGGCGCGCCAACATCAGAGAGCGCCACCCCTGAA |
| | AGTGGTCCCGGGAGCGAGCCAGCCACATCTGGGTCGGAAACGCCAGGCACAAGTGAGTCTGCAACTCCCGAGTCC |
| | GGACCTGGCTCCGAGCCTGCCACTAGCGGCTCCGAGACTCCGGGAACTTCCGAGAGCGCTACACCAGAAAGCGGA |
| | CCCGGAACCAGTACCGAACCTAGCGAGGGCTCTGCTCCGGGCAGCCCAGCCGGCTCTCCTACATCCACGGAGGAG |
| | GGCACTTCCGAATCCGCCACCCCGGAGTCAGGGCCAGGATCTGAACCCGCTACCTCAGGCAGTGAGACGCCAGGA |
| | ACGAGCGAGTCCGCTACACCGGAGAGTGGGCCAGGGAGCCCTGCTGGATCTCCTACGTCCACTGAGGAAGGGTCA |
| | CCAGCGGGCTCGCCCACCAGCACTGAAGAAGGTGCCTCGAGCCCGCCTGTGCTGAAGAGGCACCAGCGAGAAATT |
| | ACCCGGACCACCCTCCAATCGGATCAGGAGGAAATCGACTACGACGACACCATCTCGGTGGAAATGAAGAAGGAA |
| | GATTTCGATATCTACGACGAGGACGAAAATCAGTCCCCTCGCTCATTCCAAAAGAAAACTAGACACTACTTTATC |
| | GCCGCGGTGGAAAGACTGTGGGACTATGGAATGTCATCCAGCCCTCACGTCCTTCGGAACCGGGCCCAGAGCGGA |
| | TCGGTGCCTCAGTTCAAGAAAGTGGTGTTCCAGGAGTTCACCGACGGCAGCTTCACCCAGCCGCTGTACCGGGGA |
| | GAACTGAACGAACACCTGGGCCTGCTCGGTCCCTACATCCGCGCGGAAGTGGAGGATAACATCATGGTGACCTTC |
| | CGTAACCAAGCATCCAGACCTTACTCCTTCTATTCCTCCCTGATCTCATACGAGGAGGACCAGCGCCAAGGCGCC |
| | GAGCCCCGCAAGAACTTCGTCAAGCCCAACGAGACTAAGACCTACTTCTGGAAGGTCCAACACCATATGGCCCCG |
| | ACCAAGGATGAGTTTGACTGCAAGGCCTGGGCCTACTTCTCCGACGTGGACCTTGAGAAGGATGTCCATTCCGGC |
| | CTGATCGGGCCGCTGCTCGTGTGTCACACCAACACCCTGAACCCAGCGCATGGACGCCAGGTCACCGTCCAGGAG |
| | TTTGCTCTGTTCTTCACCATTTTTGACGAAACTAAGTCCTGGTACTTCACCGAGAATATGGAGCGAAACTGTAGA |
| | GCGCCCTGCAATATCCAGATGGAAGATCCGACTTTCAAGGAGAACTATAGATTCCACGCCATCAACGGGTACATC |
| | ATGGATACTCTGCCGGGGCTGGTCATGGCCCAGGATCAGAGGATTCGGTGGTACTTGCTGTCAATGGGATCGAAC |
| | GAAAACATTCACTCCATTCACTTCTCCGGTCACGTGTTCACTGTGCGCAAGAAGGAGGAGTACAAGATGGCGCTG |
| | TACAATCTGTACCCCGGGGTGTTCGAAACTGTGGAGATGCTGCCGTCCAAGGCCGGCATCTGGAGAGTGGAGTGC |
| | CTGATCGGAGAGCACCTCCACGCGGGGATGTCCACCCTCTTCCTGGTGTACTCGAATAAGTGCCAGACCCCGCTG |
| | GGCATGGCCTCGGGCCACATCAGAGACTTCCAGATCACAGCAAGCGGACAATACGGCCAATGGGCGCCGAAGCTG |
| | GCCCGCTTGCACTACTCCGGATCGATCAACGCATGGTCCACCAAGGAACCGTTCTCGTGGATTAAGGTGGACCTC |
| | CTGGCCCCTATGATTATCCACGGAATTAAGACCCAGGGCGCCAGGCAGAAGTTCTCCTCCCTGTACATCTCGCAA |
| | TTCATCATCATGTACAGCCTGGACGGGAAGAAGTGGCAGACTTACAGGGGAAACTCCACCGGCACCCTGATGGTC |
| | TTTTTCGGCAACGTGGATTCCTCCGGCATTAAGCACAACATCTTCAACCCACCGATCATAGCCAGATATATTAGG |
| | CTCCACCCCACTCACTACTCAATCCGCTCAACTCTTCGGATGGAACTCATGGGGTGCGACCTGAACTCCTGCTCC |
| | ATGCCGTTGGGGATGGAATCAAAGGCTATTAGCGACGCCCAGATCACCGCGAGCTCCTACTTCACTAACATGTTC |
| | GCCACCTGGAGCCCCTCCAAGGCCAGGCTGCACTTGCAGGGACGGTCAAATGCCTGGCGGCCGCAAGTGAACAAT |
| | CCGAAGGAATGGCTTCAAGTGGATTTCCAAAAGACCATGAAAGTGACCGGAGTCACCACCCAGGGAGTGAAGTCC |
| | CTTCTGACCTCGATGTATGTGAAGGAGTTCCTGATTAGCAGCAGCCAGGACGGGCACCAGTGGACCCTGTTCTTC |
| | CAAAACGGAAAGGTCAAGGTGTTCCAGGGGAACCAGGACTCGTTCACACCCGTGGTGAACTCCCTGGACCCCCCA |
| | CTGCTGACGCGGTACTTGAGGATTCATCCTCAGTCCTGGGTCCATCAGATTGCATTGCGAATGGAAGTCCTGGGC |
| | TGCGAGGCCCAGGACCTGTACTGA |

1. A recombinant bacmid comprising:
a bacterial replicon;
a selectable marker sequence;
a first reporter gene comprising a first preferential target site for the insertion of a transposon;
a second reporter gene operably linked to a baculovirus-inducible promoter; and
a second preferential target site capable of mediating a site-specific recombination event.

2. The recombinant bacmid of claim 1, wherein the bacterial replicon is a low-copy number replicon, optionally wherein the low-copy number replicon is a mini-F replicon.

3. (canceled)

4. The recombinant bacmid of claim 1, wherein the selectable marker sequence comprises an antibiotic resistance gene, optionally wherein the antibiotic resistance gene is a kanamycin resistance gene.

5-8. (canceled)

9. The recombinant bacmid of claim 1, wherein the first preferential target site for the insertion of a transposon does not disrupt the reading frame of the first reporter gene.

10-15. (canceled)

16. The recombinant bacmid of claim 1, wherein the second preferential target site comprises a LoxP site or a variant thereof, and wherein the site-specific recombination event is mediated by a Cre recombinase.

17-18. (canceled)

19. The recombinant bacmid of claim 1, further comprising:
a heterologous sequence inserted into the first reporter gene, wherein the inserted heterologous sequence disrupts the reading frame of the first reporter gene.

20-27. (canceled)

28. The recombinant bacmid of claim 20, wherein the protein is a Rep protein isolated from a genome of a member of the viral family Parvoviridae, optionally wherein the Rep protein is selected from the group consisting of a B19 Rep, an AAV2 Rep, a HBoV1 Rep, and a GPV Rep.

29-39. (canceled)

40. The recombinant bacmid of claim 19 comprising:
a heterologous sequence inserted into a mini-attTn7 site, wherein the heterologous sequence encodes a Rep, and wherein the inserted Rep disrupts the reading frame of the LacZα gene or functional portion thereof; and
a LoxP site or a variant thereof.

41. (canceled)

42. The recombinant bacmid of claim 40 comprising:
a bacterial replicon;
a first antibiotic resistance gene;
a heterologous sequence inserted into a mini-attTn7 site, wherein the heterologous sequence encodes a B19 Rep, and wherein the inserted B19 Rep disrupts the reading frame of the LacZα gene or functional portion thereof;
a gene encoding a fluorescent protein operably linked to a baculovirus-inducible promoter; and
a LoxP site or a variant thereof.

43. The recombinant bacmid of claim 40 comprising:
a bacterial replicon;
a first antibiotic resistance gene;
a heterologous sequence inserted into a mini-attTn7 site, wherein the heterologous sequence encodes a GPV Rep, and wherein the inserted GPV Rep disrupts the reading frame of the LacZα gene or functional portion thereof;
a gene encoding a fluorescent protein operably linked to a baculovirus-inducible promoter; and
a LoxP site or a variant thereof.

44. The recombinant bacmid of claim 40 comprising:
a bacterial replicon;
a first antibiotic resistance gene;
a heterologous sequence inserted into a mini-attTn7 site, wherein the heterologous sequence encodes an AAV2 Rep, and wherein the inserted AAV2 Rep disrupts the reading frame of the LacZα gene or functional portion thereof;
a gene encoding a fluorescent protein operably linked to a baculovirus-inducible promoter; and
a LoxP site or a variant thereof.

45-94. (canceled)

95. A recombinant bacmid comprising:
a first heterologous sequence inserted into a first reporter gene, wherein the inserted heterologous sequence disrupts the reading frame of the first reporter gene;
a first preferential target site capable of mediating a site-specific recombination event;
a multiple cloning site comprising a second heterologous sequence; and
a second preferential target site capable of mediating a site-specific recombination event.

96.-138. (canceled)

139. The recombinant bacmid of claim 95 comprising:
a sequence encoding a Rep, wherein the inserted Rep sequence disrupts the reading frame of the LacZα gene or functional portion thereof; and
a heterologous sequence comprising, from 5′ to 3′:
a wild-type or truncated 5′ inverted terminal repeat derived from a first genome of a member of the viral family Parvoviridae;
a sequence encoding a protein;
one or more expression control sequences operably linked to the sequence encoding a protein; and
a wild-type or truncated 3′ inverted terminal repeat derived from a second genome of a member of the viral family Parvoviridae.

140.-150. (canceled)

151. The recombinant bacmid of claim 95 comprising:
a sequence encoding a Rep protein, wherein the Rep disrupts the reading frame of a reporter gene or functional portion thereof; and
a heterologous sequence comprising the nucleotide sequence of SEQ ID NO: 20.

152. The recombinant bacmid of claim 95, wherein the heterologous sequence comprises the nucleotide sequence of SEQ ID NO: 19.

153. The recombinant bacmid of claim 95 comprising:
a sequence encoding a Rep protein, wherein the Rep disrupts the reading frame of a reporter gene or functional portion thereof; and
a heterologous sequence comprising the nucleotide sequence of SEQ ID NO: 29.

154. A host cell comprising the recombinant bacmid of claim 1.

155-157. (canceled)

158. A host cell comprising the recombinant bacmid of claim 95.

159-160. (canceled)

161. A method of generating a recombinant baculovirus comprising transfecting the recombinant bacmid of claim 95 into an insect cell under appropriate conditions.

162. A method of generating a closed ended DNA (ceDNA) molecule comprising:
infecting an insect cell with a recombinant baculovirus comprising the recombinant bacmid of claim 1.

163. A method of generating a closed ended DNA (ceDNA) molecule comprising:
transfecting the recombinant bacmid of claim 139 into an insect cell under appropriate conditions to generate a recombinant baculovirus; and
infecting a second insect cell with the recombinant baculovirus under appropriate conditions to generate a ceDNA molecule.

164.-181. (canceled)

182. A stable cell line comprising the nucleic acid sequence of claim 170, wherein the nucleic acid sequence is stably integrated in the genome of the stable cell line.

183.-187. (canceled)

188. A set of recombinant bacmids comprising a first bacmid and a second bacmid,
wherein the first bacmid comprises a sequence encoding a Rep inserted into a mini-attTn7 site, wherein the inserted Rep disrupts the reading frame of a reporter gene or functional portion thereof; and
wherein the second bacmid comprises a heterologous sequence, wherein the heterologous sequence comprises from 5′ to 3′:
a wild-type or truncated 5′ inverted terminal repeat (ITR) derived from a first genome of a member of the viral family Parvoviridae;
a sequence encoding a protein;
one or more expression control sequences operably linked to the sequence encoding a protein; and
a wild-type or truncated 3′ inverted terminal repeat (ITR) derived from a second genome of a member of the viral family Parvoviridae.

189.-192. (canceled)

193. A method of generating a closed ended DNA (ceDNA) molecule comprising:
infecting an insect cell with a recombinant baculovirus comprising the set of recombinant bacmids of claim 188.

194. A method of generating a closed ended DNA (ceDNA) molecule comprising:
transfecting the set of recombinant bacmids of claim 188 into an insect cell under appropriate conditions to generate a recombinant baculovirus; and
infecting a second insect cell with the recombinant baculovirus under appropriate conditions to generate a ceDNA molecule.

195. The recombinant bacmid of claim 40 comprising:
a bacterial replicon;
a first antibiotic resistance gene;
a sequence encoding a HBoV1 Rep, wherein the inserted HBoV1 Rep sequence disrupts the reading frame of the LacZα gene or functional portion thereof;
a gene encoding a fluorescent protein operably linked to a baculovirus-inducible promoter; and
a LoxP site or a variant thereof.


SEQUENCE LISTING (30 sequences)

SEQ ID NO: 1 - DNA, 248 bp, Artificial Sequence
  Synthetic polynucleotide sequence.
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 2 - DNA, 383 bp, Artificial Sequence
  Synthetic polynucleotide sequence.
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 3 - DNA, 282 bp, Artificial Sequence
  Synthetic polynucleotide sequence.
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 4 - DNA, 444 bp, Artificial Sequence
  Synthetic polynucleotide sequence.
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 5 - DNA, 145 bp, Artificial Sequence
  Synthetic polynucleotide sequence.
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - DNA, 145 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 130 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 119 bp, Artificial Sequence
  Synthetic polynucleotide sequence.
aggaacccctagtgatggagttggccactccctctctgcgcgctcgctcgctcactgaggccgggcgaccaaaggtcgcccgacgcccgggcggcctcagtgagcgagcgagcgcgcag

SEQ ID NO: 9 - DNA, 4860 bp, Artificial Sequence
  Synthetic polynucleotide sequence.
ggatccttacaatacttcctgtataacctctctaactacattaggagtacaatccacgtcaattacacgtttagctatttttctaattttgtaatgtttatcgtagagtttttcgttaatacattgaatagccaacaagggatttgggtgcacaccgtcatagagtacttccatgtcgtcttcaaagcgcatttttcgcttgcgaaaatgccgctcttggcccaaaacaaaagcgagtttgatgcggtcgtcgatgcgttccgaaaatacggccaaatgctggtgtttggtgatgtcgcgcggaaacgtcaccgtgccatttttgctttccgccacgacggcggttttcaatttttcggccgactgcagcatgttaagtttggcgtcgagttcgtgcaaacgcaattcaaactgctcaaacctgttgcccacctcgttcttgaacgtctcgtgggtgaccataaatttttcgctgtttgcattcagtttctttacatgttttaaaacagattcaatcttgtcgcgcaaatcatcacgctcgccttcagtttgaatgtgcagcaacgcgttgcttttgttggcaaaatttaaccgcatcaaaatttccaacaacccgtgcttggtcgcgaacaatgcgcccaacgagttgagatcgcgtttggatctctgtttgtgaaaaacaatttcgtttaaatggtaaacttgatcgccgtcccaattgcaatcaagtatgtcgtcgtgcgcaatttcaagacctttgcaaaaatctatcacattgtagcattttgcgttcgtgtcgctgtgcacgtatctgtacttgaaactgtgcgtgttgcatttgaatgagtcccatttaacgatgtgcgaccattgttgggcgtttatgtggtactttttgtagtcgtctgcattgaaccgatcttcggcggcgatggcgtcgttgtcgttgtcaccggaccacatccaccagttccataaccaggatagcattgctttagcttgtctagcaattcctttgttatacaacgagaaaatttcgttcccttataattatagctgtacggtgcgcgtatttgtttgttaacgttacaaaaaatatccctgtccacgtccggccaatactgcaacgtgagcgcgtccaagtttgaatcttgcatatgcggaacgtacaaacgtacggcctctctcacacaatgcgcaaaactgcccggctgaatgtaatcactgtccaactttgcaggtttctcgaaagccttgtaccgatgcacgcgaacattttgagcggacgtgattttaaacttgtcggtgaattttaaccacaaatgaaatccacggttgccggtatacatgactcttgacacgttctcttccgtgtaaaacaacagaaacgccgtggcgccaatgtaaattttcagcattaaatcgtgttcgtcaacataatttttgtaatcggcgtctacgacccattccctgccgccgccgtcgtccaacggtttgacgtgcacgtcggacactttgttttgcacaatataactatacaattgtgcggaggtatcaaaatatctgtcggcgtgaatccagcgcgcgttgaccgtcatgaacgcgtacttgcggctgtcgttgtacgcaatggcgtcccacatcatgtcgacgcgcttctgcgtataattgcacactaacatgttgcccttacaattcgcgcgcacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaacttcacaccatgtctctatccaaacaggtcctcccgcatgacgtcaaaatgaggtaccacatggacggctgcgtcaatggtcatagcttcactatcgaaggcgaaggtgccggcaaaccgtacgaggggaagaaaatcttggagctaagagtgacaaagggcggtcctctgccattcgcattcgacatcctgtccagcgtgttcacttatggaaaccgttgcttttgtgagtacccggaggacatgccggattactttaagcagagcctgcccgagggacattcgtgggaacgaacgctcatgttcgaggatggaggctgcggcaccgcgtctgctcacatcagcctggacaaaaactgcttcgtgcacaagagcactttccacggtgtgaacttccccgcaaacggtccagtgatgcagaagaaagccatgaactgggagccctcatccgaactgatcaccgcatgtgatggaatccttaagggggatgtaaccatgttcctgctgctggagggcggtcaccggctcaagtgtcagttcactacctcatacaaagcccacaaagcggtgaagatgccacccaatcacatcatcgagcatgtactcgtcaaaaaggaggtcgcggatggattccagatccaagagcacgcagtggcaaaacatttcacagtggacgtgaaagaaacctaattaacgcgttagcattaaaattaaacagtttaataaataaataatatttaatttattttttgttttattatctattttgtaacaacaacaatatcagtcttttgatcatgataacttcgtatagcatacattatacgaagttatatcatcctagcgccaatgtaccgtattcaaattactacatgtataaatctgtgttttctattgtaatgaatcacttaacacacttttaattacgtcaataaatgttattcaccattatttacctggtttttttgagaggggctttgtgcgactgcgcacttccagcctttataaacgctcaccaaccaaagcaggtcattattgtgccaggacgttcaaaggcgaaacatcgaaatggagtctgttcaaacgcgcttatgtgccagtagcaatcaatttgctccgttcaaaaagcgccagcttgccgtgccggtcggttctgtgaacagtttgacacacaccatcacctccaccaccgtcaccagcgtgattccaaaaaattatcaagaaaaacgtcagaaaatatgccacataatatcttcgttgcgtaacacgcacttgaatttcaataagatacagtctgtacataaaaagaaactgcggcatttgcaaaatttgctaagaaaaaagaacgaaattattgccgagttggttagaaaacttgaaagtgcacagaagaagacaacgcacagaaatattagtaaaccagctcattggaaatactttggagtagtcagatgtgacaacacaattcgcacaattattggcaacgaaaagtttgtaaggagacgtttggccgagctgtgcacattgtacaacgccgagtacgtgttttgccaagcacgcgccgatggagacaaagatcgacaggcactagcgagtctgctgacggcggcgtttggttcgcgagtcatagtttatgaaaatagtcgccggttcgagtttataaatccggacgagattgctagtggtaaacgtttaataattaaacatttgcaagatgaatctcaaagtgatattaacgcctattaatttgaaaggtgaggaagagcccaattgcgttgagcgcattaccataatgccatgtattttaatagatactgagatctgtttaaatgtcagatgccgttctccttttgccaaattcaaagtattgattattgtagatggctttgatagcgcttatattcaggctaccttttgtagcattagcgatagtgtaacaattgttaacaaatctaacgaaaagcatgtaacgtttgacgggtttgtaaggccggacgatgaaggtacaacaatgccttatgtcattggaccattatattctgtcgacgctgctgtcgccgaccgtaaagtgaaggacgtggtggattcaattcaaaaccaacagacaatgttaaaagtatttattaacgaggctaatgtgtataacaaatggaatatgcttaaaggtttaatttataataataacaatgaatctgttttagtaaataatgtagtaaaatttataaaggtagataaaaattataatattaataaaaaaaataatgttactaaatgggttcctgcgttaaattattttacgggtagacagctattaactattttatttatttttaaatttaaataaatgtattgttagaaaattgtgttgttttattagtataacgaaaaaatacatgacataaaccgcttccaattttggtcacacaaactcttgtgtggatagtttacgtaatgagttaaataggcgggcagttgtccgctaaacgtgtcggtggtcaagtagatgtgcattaatttacgacaacccaaagcggggccgcttatgtcaagtatttttttcacaaaattggtaatggtttcgttttgttccttgtacaaacacatgtcggtgtgatcgttgacgcacgagttgtacgattccgccggcaggttggcaaacaagcgcttgagatgcttgagtctgcgttcaattttataatcaaacttgttggtgaaaatgtctttcagcaagcacattaactggtcgttcaaaacgcgctgcaacgacgacaccaacacatgatattcgtttccaaaaagcgaaaaatttttgatgcagcggtccgcgttgaagggtcgtttcataatgcgcacgttgacaaaaaacacgttgaaagacagcggggctgtggttattttaacgccgttgtcggtatactcgtcgacgccgtctgcgcttgttatgtcggccggggatcc

SEQ ID NO: 10 - DNA, 3954 bp, Artificial Sequence
  Synthetic polynucleotide sequence.
attgctgagcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgggtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaagttaaccgaccatggatttgtaaaaaaaaaataaataaaaatggtgtccaagggcgaggaactgttcaccggtgtcgtgcccatcctggtcgaactggacggcgacgtgaacggtcacaagttctccgtgtctggcgaaggcgagggcgacgctacctacggaaagctgaccctgaagttcatctgcaccaccggcaagctgcccgtgccttggcctaccctggtcaccactctgacctacggtgtccagtgcttctcccgttaccccgaccacatgaagcagcacgatttcttcaagtccgctatgcccgagggttacgtgcaagagcgtaccatcttcttcaaggacgacggcaactacaagacccgtgctgaagtgaagttcgaaggcgacaccctcgtgaaccgtatcgagctgaagggtatcgacttcaaggaagatggaaacatcctgggccacaagctcgagtacaactacaactcccacaacgtgtacatcatggccgacaagcaaaagaacggcatcaaagtgaacttcaagatccgccacaacatcgaggacggttccgtgcagctggctgaccactaccagcagaacacccccatcggcgacggtcctgtgctgctgcctgacaaccactacctgtccacccagtccgctctgtccaaggaccccaacgagaagcgtgaccacatggtgctgctcgagttcgtgaccgctgctggtatcaccctgggcatggacgagctgtacaagtaatcgcgagcccgatgtaaacgccacaattgtgtttgttgcaaataaacccatgattatttgattaaaattgttgttttctttgttcatagacaatagtgtgttttgcctaaacggtttaaactggccaagagtatgcccgttcctcctacttttaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccctgcagggctcttcactagtgaagagcactagtcctgcaggaaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgatcgataacttcgtatagcatacattatacgaagttat

SEQ ID NO: 11 - DNA, 2044 bp, Artificial Sequence
  Synthetic polynucleotide sequence.
attaggatccgccgccatggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcggggtgcttgtacttttttcaagtagaatgtaacaaatttgaagaaggctatcatattcatgtggttattggggggccagggttaaaccccagaaacctcacagtgtgtgtagaggggttatttaataatgtactttatcaccttgtaactgaaaatgtaaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgacactagtagtgatgctggggagtctagcggcacaggggcagaggttgtgccaattaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctacaagcacatttctattgcatacagactttgagcaggttatgtgtattaaagacaataaaattgttaaattgttactttgtcaaaactatgaccccctattagtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgaatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagctcagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaagcggccgcatta

SEQ ID NO: 12 - DNA, 1912 bp, Artificial Sequence
  Synthetic polynucleotide sequence.
attaggatccgccgccctggcactttctaggcctcttcagatttcttctgataaattctacgaagttattattagattatcatcggatattgatcaagacgtccccggtctgtctcttaactttgtagagtggctttctaccggagtttgggagcccacgggcatctggaacttagagcacgtgaatctaccgttagtgaccttggcagagaagatcaagaacattttcatacaaaggtggaatcagttcaaccaggacgaaacggacttcttctttcaactggaagaaggcagtgagtacattcatcttcattgctgtattgcccagggcaacgtacggtcttttgttctcgggagatatttatctcagataaaagactctatcataagagacgtatacgaagggaaacaaatcaagatccccgattggtttgctattactaaaaccaagaggggaggacagaataagaccgtgactgcagcatacatactgcattaccttattcctaaaaagcaacctgaactgcagtgggcctttaccaatatgcctttattcactgctgctgctctttgtctgcaaaagcggcaagaattgctggatgcatttcaagaaagtgatttggctgcccctttacctgatcctcaagcatcaactgtggcaccgcttatttccaacagagcggcaaagaactatagcaaccttgttgattggctcattgaaatggggataacatctgagaagcaatggctcactgagaaccgagagagctacagaagctttcaagcaacttcttcaaataatagacaagtgaaagctgcactggaaaatgcccgtgctgaaatgttattgacaaagactgcaactgattacctgataggaaaagaccctgtcctggatataactaagaatagggtctatcaaattctgaaaatgaataactacaaccctcaatacataggaagtatcctgtgcggctgggtgaagagagagttcaacaaaagaaacgccatatggctctacggacctgccaccaccgggaagaccaacattgcagaagctattgcccatgctgtacccttctatggctgtgttaactggactaatgagaactttccttttaatgattgtgttgataaaatgctgatttggtgggaggagggaaaaatgactaataaggttgttgaatctgcaaaagcaattttgggagggtctgctgtccgggtagaccagaaatgtaaaggatctgtttgtattgaacctactcctgtaattattactagtaatactgatatgtgtatgattgttgatggcaactctactacaatggaacatagaataccattagaggagcgtatgtttcaaattgtcctatcacataaattggagccttcttttggaaaaatttctaaaaaagaagtcagagaatttttcaaatgggccaatgacaatctagttcctgttgtgtctgagttcaaagtccgaactaatgaacaaaccaacttgccagagcccgttcctgaacgagcgaacgagccggaggagcctcctaagatctgggctcctcctactagggaggagttagaagagcttttaagagccagcccagaattgttctcatcagtcgctccaattcctgtgactcctcagaactcccctgagcctaagagaagcaggaacaattaccaggtacgctgcgctttgcatacttatgacaattctatggatgtatttgaatgtatggaatgtgagaaagcaaactttcctgaatttcaacctctgggagaaaattattgtgatgaacatgggtggtatgattgtgctatatgtaaagagttgaaaaatgaacttgcagaaattgagcatgtgtttgagcttgatgatgctgaaaatgaacaataagcggccgcatta

SEQ ID NO: 13 - DNA, 1894 bp, Artificial Sequence
  Synthetic polynucleotide sequence.
attagcggccgcgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataagcatgcatta

SEQ ID NO: 14 - DNA, 2033 bp, Artificial Sequence
  Synthetic polynucleotide sequence.
attaagcttccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgggtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacgcggccgcatttgtaaaaaaaaaataaataaaaatgatcgagcaggacggcctgcacgctggttctccagctgcttgggtcgagcgtctgttcggttacgactgggctcagcagaccatcggttgctccgacgctgctgtgttccgtctgtccgctcagggtcgtcccgtgctgttcgtcaagaccgacctgtccggtgctctgaacgagctgcaggacgaggctgctcgtctgtcctggctggctaccactggtgtcccttgcgctgctgtcctggacgtggtcactgaggctggtcgtgactggctgctgctgggagaagtgcctggacaggacctgctgtccagccacctggctccagctgagaaggtgtccatcatggctgacgctatgcgtcgtctgcacaccctggaccctgctacctgccccttcgaccaccaagctaagcaccgtatcgagcgtgctcgtacccgtatggaagctggcctggtggaccaggacgacctggacgaagaacaccagggactggcccctgctgagctgttcgctcgtctgaaggctcgtatgcccgacggcgaggacctggtggttactcacggcgacgcttgcctgcccaacatcatggtcgagaacggtcgtttctccggtttcatcgactgcggtcgtctgggtgtcgctgaccgttaccaggatatcgctctggctacccgtgatatcgctgaggaactgggtggcgagtgggctgacagattcctggtgctgtacggtatcgctgctcccgactcccagcgtatcgctttctaccgtctgctggacgagttcttctaagccccttgtaaacgccacaattgtgtttgttgcaaataaacccatgattatttgattaaaattgttgttttctttgttcatagacaatagtgtgttttgcctaaacgggtacc

SEQ ID NO: 15 - DNA, 592 bp, Artificial Sequence
  Synthetic polynucleotide sequence.
attaggcgcgcctacgtatactcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaagcggccgcggatccatta

SEQ ID NO: 16 - DNA, 126 bp, Artificial Sequence
  B19 Minimal ITR
ccaaatcagatgccgccggtcgccgccggtaggcgggacttccggtacaggcgcgccgctgtaccggaagtcccgcctaccggcggcgaccggcggcatctgatttggtgtcttcttttaaatttt

SEQ ID NO: 17 - DNA, 333 bp, Artificial Sequence
  GPV DELTA 120 ITR
ctagcgagctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcgcatgctcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtgt

SEQ ID NO: 18 - DNA, 261 bp, Artificial Sequence
  GPV DELTA 186 ITR
ctagcgagctcattggagggttcgttcgttcgaaccagccaatcaggggagggggaagtgacgcaagttccggtcacatgcttccggtgacgcacatccggtgacgtagttcgcatgcgaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtgt

SEQ ID NO: 19 - DNA, 7891 bp, Artificial Sequence
  V2.0 Human codon optimized mTTR-Intron-BDD- FVIIIXTEN-WPRE-bGHPoly expression cassette
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctaggaattctcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaattactcgaggccaccatgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaagcggccgctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgcctaggcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagaccatgggcgcgccaggcctgtcgacgcccgggcggtaccgcgatcgctcgcgacgcataaag

SEQ ID NO: 20 - DNA, 4824 bp, Artificial Sequence
  Nucleotide sequence encoding B-domain deleted (BDD) codon-optimized human Factor VIII (BDDcoFVIII) fused with 144 amino acid XTEN
atgcagattgaactgtccacttgcttcttcctgtgcctcctgcggttttgcttctcggccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccgacctccgaaagcgccaccccagagtcaggacctggctcggaaccggctacctcgggctcagagacaccggggacttccgagtccgcaacccccgagagtggacccggatccgaaccagcaacctcaggatcagaaaccccgggaacttcggaatccgccactcccgagtcgggaccaggcacctccactgagccttccgagggaagcgcccccggatcccctgctggatcccctaccagcactgaagaaggcacctcagaatccgcgacccctgagtccggccctggaagcgaacccgccacctccggttccgaaacccctgggactagcgagagcgccactccggaatcgggcccaggaagccctgccggatccccgaccagcaccgaggagggaagccccgccgggtcaccgacttccactgaggagggagcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 21 - DNA, 330 bp, Artificial Sequence
  A1MB2 ENHANCER
ggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccaccttcgatggccccaggttaatttttaaaaagcagtcaaaggtcaaagtggcccttggcagcatttactctctctattgactttggttaataatctcaggagcacaaacattcctggaggcaggagaagaaatcaacatcctggacttatcctctgggcctctccccacc

SEQ ID NO: 22 - DNA, 345 bp, Artificial Sequence
  mTTR promoter
gatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctag

SEQ ID NO: 23 - DNA, 1489 bp, Artificial Sequence
  CHIMERIC INTRON
tcaggagcacaaacattcctggaggcaggagaagaaatcaacatcctggacttatcctctgggcctctccccaccgatatctacctgctgatcgcccggcccctgttcaaacatgtcctaatactctgtcggggcaaaggtcggcagtagttttccatcttactcaacatcctcccagtgtacgtaggatcctgtctgtctgcacatttcgtagagcgagtgttccgatactctaatctcccggggcaaaggtcgtattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttattgacggcttgtttcttttctgtggctgcgtgaaagccttgaggggctccgggaaggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccttgttcttgccttcttctttttcctacagctcctgggcaacgtgctggttattgtgctgtctcatcattttggcaaagaatta

SEQ ID NO: 24 - DNA, 595 bp, Artificial Sequence
  WPRE
tcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctg

SEQ ID NO: 25 - DNA, 211 bp, Artificial Sequence
  bGHpA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 26 - DNA, 140 bp, Artificial Sequence
  HBoV1 5′ ITR
gtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccac

SEQ ID NO: 27 - DNA, 200 bp, Artificial Sequence
  HBoV1 3′ ITR
ttgcttatgcaatcgcgaaactctatatcttttaatgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcataagcaa

SEQ ID NO: 28 - DNA, 4317 bp, Artificial Sequence
  Nucleotide sequence of coFVIII-6
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 29 - DNA, 4335 bp, Artificial Sequence
  NT_BDDcoFVIII_noXTEN
gccacccgccggtattacttaggtgctgtggaactgagctgggactacatgcagtccgacctgggagaactgccggtggacgcgagattcccacctagagtcccgaagtccttcccattcaacacctccgtggtctacaaaaagaccctgttcgtggagttcactgaccaccttttcaatattgccaagccgcgccccccctggatgggcctgcttggtcctacgatccaagcagaggtctacgacaccgtggtcatcacactgaagaacatggcctcacaccccgtgtcgctgcatgctgtgggagtgtcctactggaaggcctcagagggtgccgaatatgatgaccagaccagccagagggaaaaggaggatgacaaagtgttcccgggtggcagccacacttacgtgtggcaagtgctgaaggaaaacgggcctatggcgtcggaccccctatgcctgacctactcctacctgtcccatgtggaccttgtgaaggatctcaactcgggactgatcggcgccctcttggtgtgcagagaaggcagcctggcgaaggaaaagactcagaccctgcacaagttcattctgttgtttgctgtgttcgatgaaggaaagtcctggcactcagaaaccaagaactcgctgatgcaggatagagatgcggcctcggccagagcctggcctaaaatgcacaccgtcaacggatatgtgaacaggtcgctccctggcctcatcggctgccacagaaagtccgtgtattggcatgtgatcggcatgggtactactccggaagtgcatagtatctttctggagggccataccttcttggtgcgcaaccacagacaggcctcgctggaaatctcgcctatcactttcttgactgcgcagaccctccttatggaccttggacagttcctgctgttctgtcacatcagctcccatcagcatgatgggatggaggcctatgtcaaagtggactcctgccctgaggagccacagctccggatgaagaacaatgaggaagcggaggattacgacgacgacctgactgacagcgaaatggacgtcgtgcgattcgatgacgacaacagcccgtccttcatccaaattagatcagtggcgaagaagcaccccaagacctgggtgcactacattgccgccgaggaagaggactgggactacgcgccgctggtgctggcgccagacgacaggagctacaagtcccagtacctcaacaacgggccgcagcgcattggcaggaagtacaagaaagtccgcttcatggcctacactgatgaaaccttcaagacgagggaagccatccagcacgagtcaggcatcctgggaccgctcctttacggcgaagtcggggataccctgctcatcattttcaagaaccaggcatcgcggccctacaacatctaccctcacgggatcacagacgtgcgcccgctctactcccgccggctgcccaagggagtgaagcacctgaaggattttcccatcctgccgggagaaatcttcaagtacaagtggaccgtgactgtggaagatggccctaccaagtcggaccctcgctgtctgacccggtactattcctcgtttgtgaacatggagcgcgacctggcctcggggctgattggtccgctgctgatctgctacaaggagtccgtggaccagcgcgggaaccagatcatgtccgacaagcgcaacgtgatcctgttctctgtctttgatgaaaacagatcgtggtacttgactgagaatatccagcggttcctgcccaacccagcgggagtgcaactggaggacccggagttccaggcctcaaacattatgcactctatcaacggctatgtgttcgactcgctccaactgagcgtgtgcctgcatgaagtggcatactggtacattctgtccatcggagcccagaccgacttcctgtccgtgttcttctccggatacaccttcaagcataagatggtgtacgaggacactctgaccctcttcccattttccggagaaactgtgttcatgtcaatggaaaacccgggcttgtggattctgggttgccataactcggacttccggaatagagggatgaccgccctgctgaaagtgtccagctgtgacaagaataccggcgattactacgaggacagctatgaggacatctccgcttatctgctgtccaagaacaacgccattgaacccaggtccttctcccaaaacggtgcaccggcctcatccccccccgtgctgaagcggcatcaaagagagatcaccaggaccactctccagtccgatcaggaagaaattgactacgacgatactatcagcgtggagatgaagaaggaggacttcgacatctacgatgaggatgagaaccagtcccctcggagctttcagaagaaaacccgccactacttcatcgctgccgtggagcggctgtgggattacgggatgtccagctcaccgcatgtgctgcggaatagagcgcagtcaggatcggtgccccagttcaagaaggtcgtgttccaagagttcaccgacgggtccttcactcaacccctgtaccggggcgaactcaacgaacacctgggactgcttgggccgtatatcagggcagaagtggaagataacatcatggtcaccttccgcaaccaggcctcccggccgtacagcttctactcttcactgatctcctacgaggaagatcagcggcagggagccgagccccggaagaacttcgtcaagcctaacgaaactaagacctacttttggaaggtccagcatcacatggccccgaccaaagacgagttcgactgtaaagcctgggcctacttctccgatgtggacctggagaaggacgtgcactcgggactcattggcccgctccttgtgtgccatactaataccctgaaccctgctcacggtcgccaagtcacagtgcaggagttcgccctcttcttcaccatcttcgatgaaacaaagtcctggtactttactgagaacatggaacgcaattgcagggcaccctgcaacatccagatggaagatcccaccttcaaggaaaactaccggtttcatgccattaacggctacataatggacacgttgccaggactggtcatggcccaggaccagagaatccggtggtatctgctctccatgggctccaacgaaaacattcacagcattcatttttccggccatgtgttcaccgtccggaagaaggaagagtacaagatggctctgtacaacctctaccctggagtgttcgagactgtggaaatgctgcctagcaaggccggcatttggagagtggaatgcctgatcggagagcatttgcacgccggaatgtccaccctgtttcttgtgtactccaacaagtgccagaccccgctgggaatggcctcaggtcatattagggatttccagatcactgcttcggggcagtacgggcagtgggcacctaagttggcccggctgcactactctggctccatcaatgcctggtccaccaaggaacccttctcctggattaaggtggacctcctggccccaatgattattcacggtattaagacccagggtgcccgacagaagttctcctcactctacatctcgcaattcatcataatgtacagcctggatgggaagaagtggcagacctaccggggaaactccactggaacgctcatggtgtttttcggcaacgtggactcctccggcattaagcacaacatcttcaaccctccgatcattgctcggtacatccggctgcacccaactcactacagcatccggtccaccctgcggatggaactgatgggttgtgacctgaactcctgctccatgccccttgggatggaatccaaggccattagcgatgcacagatcaccgcctcttcatacttcaccaacatgttcgcgacctggtccccgtcgaaggcccgcctgcacctccaaggtcgctccaatgcgtggcggcctcaagtgaacaaccccaaggagtggctccaggtcgacttccaaaagaccatgaaggtcaccggagtgaccacccagggcgtgaagtccctgctgacctctatgtacgttaaggagttcctcatctcctcaagccaagacggacatcagtggaccctgttcttccaaaacggaaaagtcaaagtattccagggcaaccaggactccttcacccctgtggtcaacagcctggaccccccattgctgacccgctacctccgcatccacccccaaagctgggtccaccagatcgcactgcgcatggaggtccttggatgcgaagcccaagatctgtactaa

SEQ ID NO: 30 - DNA, 4824 bp, Artificial Sequence
  V1.0 expression cassette
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga